(19) United States
(12) Patent Application Publication
(10) Pub. No.: US 2022/0062340 A1
(43) Pub. Date: Mar. 3, 2022

(54) IMMUNOTHERAPEUTIC METHODS AND COMPOSITIONS

(71) Applicant: KING'S COLLEGE LONDON, London (GB)

(72) Inventors: Rimma GOLDBERG, Greater London (GB); Graham LORD, Greater London (GB)

(21) Appl. No.: 17/415,485

(22) PCT Filed: Dec. 19, 2019
(86) PCT No.: PCT/GB2019/053618

§ 371 Date: Jun. 17, 2021

(30) Foreign Application Priority Data
Dec. 19, 2018 (GB) 1820746.4
Jan. 17, 2019 (GB) 1900687.3

(51) Int. Cl. A61K 35/17; A61P 37/06; A61P 1/00; C12N 5/0783

(52) U.S. Cl. CPC A61K 35/17; A61P 37/06; C12N 2501/999; C12N 5/0637; A61P 1/00

(57) ABSTRACT

This invention relates to immunotherapeutic methods involving administering immunoregulatory T cells (Tregs) with improved function to a subject. The invention also concerns modified Tregs having improved function and pharmaceutical compositions comprising the same. The improved Tregs of the invention have the capacity for increased gut-homing, amongst other improved functions. The methods and compositions of the invention are particularly useful in the treatment of immune-mediated gut disorders.

### TECHNICAL FIELD

This invention relates to immunotherapeutic methods involving administering immunoregulatory T cells (Tregs) with improved function to a subject in need thereof. The invention also concerns ex vivo expanded and modified Tregs having improved function and pharmaceutical compositions comprising the same. The improved Tregs of the invention have the capacity for increased gut-homing, amongst other improved functions. The methods and compositions of the invention are particularly useful in the treatment of immune-mediated gut disorders.

### BACKGROUND

Regulatory T cells (Tregs) are T cells which play a role in suppressing or regulating other cells in the immune system. Tregs are important in controlling the immune response to self and foreign particles (antigens) and help prevent autoimmune disease.

Crohn's Disease (CD) is a chronic, immune-mediated inflammatory bowel disease (IBD) with no known cure, resulting in significant morbidity. Goals of therapy include resolution of symptoms and mucosal healing. However, many patients have sub-optimal responses to currently available therapies. This represents a significant unmet medical need. There is good evidence from both genetic and functional studies implicating defective Treg function in the pathogenesis of inflammatory bowel disease ¹⁻⁴.

The maintenance, or indeed loss, of intestinal homeostasis hinges on the balance between inflammatory effector T-cells (Teff), which have been implicated in auto-immunity and transplant rejection, and a population of Treg⁵⁻⁷. Tregs are a unique subset of CD4+ T cells with powerful immunosuppressive action. They are defined by expression of the master transcriptional regulator FOXP3 and a set of key surface markers ⁸⁻¹⁰. Tregs serve to limit immune mediated pathology, and mice or humans lacking functional Tregs develop severe multisystem inflammatory disease, including chronic intestinal inflammation (IPEX syndrome)¹¹.

Recent advances in therapy for IBD have focused on T cell trafficking and more specifically, the diversion of effector T cells from the inflamed gut by blocking the gut specific trafficking molecule integrin α4β7¹⁷. The efficacy of this therapy would suggest that trafficking of lymphocytes to the inflamed gut is a key step in the pathogenesis of CD. Current reports suggest that there is no defect in Treg trafficking in patients with CD¹⁸ and that there is indeed a greater number of CD4⁺FOXP3+ cells in the lamina propria of CD patients compared to healthy controls (HC) ¹⁹. However, considerable evidence exists to support the hypothesis that the Tregs present in the lamina propria are locally induced and can develop IL17 secreting capabilities under pro-inflammatory conditions, which may reduce their suppressive capacity ^{20, 21}.

Tregs purified from peripheral blood (PB) of CD patients play a critical role in controlling both phenotype and expansion of auto-reactive T cells ²². Retinoic acid (RA) regulates the expression of the primary gut homing integrin α4β7 and the mechanisms by which RA controls the stability of T cell lineage commitment have previously been defined ²³. It has also been shown that that RA can induce the expression of α4β7 on normal (HC) Tregs following in vitro culture ¹³.

RA is effective at inducing the expression of integrin α4β7 and has been suggested to have an effect on improving Treg suppressive ability²⁴. However, the stabilizing effect of all-trans retinoic acid (ATRA) on Tregs has been found to be transient and serum dependent, and there are ongoing concerns about the ability of retinoic acid to also skew Tregs towards a pro-inflammatory phenotype ²⁵. Additionally, ATRA binds to the retinoic acid receptors (RARα, β, and γ) with similar affinity and their activation in the presence of this ligand is relatively non-selective ²⁶. Therefore, all RARs and RXRs will be activated within the cell, some of which may be associated with adverse off-target effects.

Given the sub-optimal responses to currently available therapies for IBD and other immune-mediated gut disorders, there remains an unmet medical need.

### SUMMARY OF THE INVENTION

The present invention provides a method for making regulatory T cells (Tregs) with improved functionality, comprising contacting Tregs derived from a subject with an immune-mediated gut disorder with at least one RARα agonist, functional analogue or derivative thereof.

Also provided are ex vivo expanded Tregs which have previously been contacted with at least one RARα agonist, functional analogue or derivative thereof prior to being administered to a subject in need thereof, and which Tregs have increased capacity for gut-homing and/or altered expression of gut-homing molecules relative to controls. The Tregs may optionally be obtainable or obtained by the methods of the invention.

The improved Treg function may be in the form of increased capacity for gut-homing and/or improved Treg retention and/or increased potency and/or wherein the Tregs are not skewed towards a pro-inflammatory phenotype.

The invention also provides modified Tregs having altered expression of a gut-homing molecule relative to controls.

Also provided are pharmaceutical compositions comprising such ex vivo expanded and/or modified Tregs.

The present invention also provides a method of treating, ameliorating or preventing the symptoms or progression of an immune-mediated gut disorder, comprising contacting Tregs previously obtained from a subject having an immune-mediated gut disorder with at least one RARα agonist, functional analogue or derivative thereof before introducing the treated Tregs into the same or different subject in need of treatment. The method of treatment may also comprise administering to a subject having an immune-mediated gut disorder ex vivo expanded and/or modified Tregs or a pharmaceutical composition comprising the same.

The present invention also provides ex vivo expanded and/or modified Tregs with improved functionality and/or RARα agonists, functional analogues and derivatives thereof for use in the treatment of an immune-mediated gut disorder.

The present invention also provides culture and/or expansion media for use in the production of ex vivo expanded Tregs, which media comprise at least one RARα agonist, functional analogue or derivative thereof.

### DETAILED DESCRIPTION

According to a first aspect of the present invention, there is provided a method for making regulatory T cells (Tregs) with improved functionality, comprising contacting Tregs derived from a subject with an immune-mediated gut disorder with at least one RARα agonist, functional analogue or derivative thereof.

The method of the invention incorporates known methods for Treg isolation, culture, expansion and infusion into patients, except that the culture and/or expansion media comprises at least one RARα agonist, functional analogue or derivative thereof.

The first step of the method involves obtaining a biological sample from a subject having an immune-mediated gut disorder. Tregs may be obtained from any suitable biological sample including, without limitation, peripheral blood, thymus, lymph nodes, spleen, bone marrow, and includes natural Treg (nTreg) cells and peripherally generated, induced Treg (iTreg) cells, which may be induced with antigen stimulation and cytokines such as TGF-β.

The immune-mediated gut disorder may be selected from, but is not limited to, inflammatory bowel disease (IBD), such as Chron's Disease (CD) and/or ulcerative colitis (UC). The immune-mediated gut disorder may be selected from, but is not limited to, celiac disease; autoimmune gastritis; colitis, such as checkpoint-related colitis (colitis associated with the treatment for solid cancers treated with checkpoint inhibitors (such as anti-CTLA4 and/or anti-PD1/PDL1/L)); treatment-resistant colitis, (for example, due to bacteria such as Clostridium difficile); and GvHD, where the gut is involved.

Tregs are suitably isolated from peripheral blood mononuclear cells (PBMCs) obtained from the subject. Suitably the subject is a mammal, preferably a human, having an immune-mediated gut disorder. Suitably the cell is matched or is autologous to the subject. In a preferred embodiment, the Tregs are isolated from peripheral blood mononuclear cells (PBMCs) obtained from a subject and is matched or is autologous to the subject to be treated.

As used herein, the term “Treg” refers to a T cell with immunosuppressive function. Suitably, the Treg to be isolated from the biological sample is a T cell which expresses the markers CD4, CD25 and FOXP3 (CD4+CD25+FOXP3+). “FOXP3” is the abbreviated name of the forkhead box P3 protein. FOXP3 is a member of the FOX protein family of transcription factors and functions as a master regulator of the regulatory pathway in the development and function of regulatory T cells.

Suitably, the Treg may be identified using the cell surface markers CD4 and CD25 in the absence of or in combination with low-level expression of the surface protein CD127 (CD4+CD25+CD127− or CD4+CD25+CD127low).

The Treg may be a CD4+CD25+FOXP3+ T cell.

The Treg may be a CD4+CD25+CD127−/low T cell.

The Treg may be a CD4+CD25+FOXP3+CD127−/low T cell.

The Treg may be a CD4+CD25+CD127−CD45RA+ T cell.

The Treg may be a CD4+CD25+CD127lowCD45RA+ T cell.

The Treg may be a CD4+CD25+CD127lowCD45RA-CD45RO+ T cell.

The Treg may be a CD4+CD25+CD127lowCD45RA−CD45RO+ T cell.

Suitably, the Treg may be a natural Treg. As used herein, the term “natural T reg” means a thymus-derived Treg. Natural Tregs are CD4+CD25+FOXP3+ Helios+ Neuropilin 1+. Compared with iTregs, nTregs have higher expression of PD-1 (programmed cell death-1, pdcd1), neuropilin 1 (Nrp1), Helios (Ikzf2), and CD73. nTregs may be distinguished from iTregs on the basis of the expression of Helios protein or Neuropilin 1 (Nrp1) individually.

Further suitable Tregs include, but are not limited to, Tr1 cells (which do not express Foxp3, and have high IL-10 production); CD8+FOXP3+ T cells; and γδ 5 FOXP3+ T cells.

In contrast, effector T cells (Teffs) were identified as, for example: CD4+CD25−FOXP3−CD127+.

Tregs may be isolated/purified using any convenient separation or cell sorting techniques based on Treg-specific cell markers, such as flow cytometry by any convenient method, one example being fluorescence-activated cell sorting (FACS). Commercially available kits may be used for such isolation and purification and include, without limitation, Miltenyi Treg kit with Auotmacs, ClinMACS, and the like.

The Tregs so-obtained are then cultured and expanded ex vivo in the presence of at least one RARα agonist. Other components which may be used in a Treg expansion protocol include, but are not limited to rapamycin, TGFβ, interleukins (such as IL-2 or IL-15) and activators, such as anti-CD3 and/or anti-CD28. As used herein, an “activator” stimulates a cell, causing the cell to proliferate. Preferably the interleukin is interleukin-2 (IL-2) and is present at a high dose, IL-2 being important for the homeostasis of Tregs (generation, proliferation, survival), as well as for their suppressive function and phenotypic stability. Preferably the Tregs are cultured and expanded ex vivo in the presence of at least one RARα agonist, rapamycin and IL-2 (at a high dose).

The term “RARα agonist” as defined herein is taken to mean any agent that activates RAR or sustains retinoic acid so that its activity at RAR increases. This includes both substances that initiate a physiological response when combined with a receptor, as well as substances that prevent the catabolism (or breakdown) of retinoids (for example, retinoic acid), allowing the signal from retinoic acid itself to increase. As a non-limiting list, RARα agonists include, but are not limited to ATRA, RAR568, AM580, AM80 (tamibarotene), RX-195183, BMS753, BD4, AC-93253, and AR7.

Additional RARα agonists include those provided or defined in US 2012/0149737, which is incorporated herein by reference for its teaching and definition of the chemical structure of additional RARα agonists.

For example, an RARα agonist may include a compound of the following formula, or a pharmaceutically acceptable salt thereof:

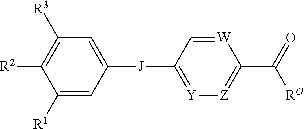

wherein:

—R¹ is independently —X, —R^{X}, —O—R^{X}, —O—R^{A}, —O—R^{C}, —O-L-R^{C}, —O—R^{AR}, or —O-L-R^{AR};

—R² is independently —X, —R^{X}, —O—R^{X}, —O—R^{A}, —O—R^{C}, —O-L-R^{C}, —O—R^{AR}, or —O-L-R^{AR};

—R³ is independently —X, —R^{X}, —O—R^{X}, —O—R^{A}, —O—R^{C}, —O-L-R^{C}, —O—R^{AR}, or —O-L-R^{AR};

with the proviso that —R¹, —R², and —R³ are not all —O—R^{A}

and/or with the proviso that —R¹ and —R² (or —R² and —R³) may be joined together to form an optionally substituted 5- or 6-membered ring R^{D};

wherein:

each —X is independently —F, —Cl, —Br, or —I;

each —R^{A} is saturated aliphatic C₁₋₆alkyl;

each —R^{X} is saturated aliphatic C₁₋₆haloalkyl;

each —R^{C} is saturated C₃₋₇cycloalkyl;

each —R^{AR} is phenyl or C₅₋₆heteroaryl;

each -L- is saturated aliphatic C₁₋₃alkylene;

and wherein:

-J- is —C(═O)—NR^{N}— or —NR^{N}—C(═O)—;

—R^{N} is independently —H or —H or —R^{NN};

—R^{NN} is saturated aliphatic C₁₋₄alkyl;

═Y— is ═CR^{Y}— and —Z═ is —CR^{Z}═;

—R^{Y} is —H;

—R^{Z} is independently —H or —R^{ZZ};

—R^{ZZ} is independently —F, —Cl, —Br, —I, —OH, saturated aliphatic C₁₋₄alkoxy, saturated aliphatic C₁₋₄alkyl, or saturated aliphaticC₁₋₄haloalkyl;

═W is ═CR^{w};

—R^{w} is —H;

—R^{O} is independently —OH, —OR^{E}, —NH₂, —NHR^{T1}, —NR^{T1}R^{T1} or —NR^{T2}R^{T3};

—R^{E} is saturated aliphatic C₁₋₆alkyl;

each —R^{T1} is saturated aliphatic C₁₋₆alkyl;

—NR^{T2}R^{T3} is independently azetidino, pyrrolidino, piperidino, piperizino, N—(C₁₋₃alkyl) piperizino, or morpholino;

optionally with the proviso that the compound is not a compound selected from the following compounds, and salts, hydrates, and solvates thereof:

4-(3,5-dichloro-4-ethoxy-benzoylamino)-benzoic acid (PP-02); and/or

4-(3,5-dichloro-4-methoxy-benzoylamino)-benzoic acid (PP-03).

In various embodiments, —R¹ may be —X or —O—R^{A}.

In various embodiments, —R² may be —X or —O—R^{A}.

In various embodiments, —R³ may be —X or —O—R^{A}.

In various embodiments, two of —R¹, —R² and —R³ may independently be —O—R^{A} with the remaining —R¹, —R² or —R³ being —X.

In various embodiments, —X may be —Cl.

In various embodiments, —R^{A} may be methyl, ethyl, propyl (n-propyl or iso-propyl), butyl (n-butyl, iso-butyl, sec-butyl or tert-butyl), pentyl (n-pentyl, iso-pentyl or neo-pentyl) or hexyl, for example methyl, ethyl or propyl (n-propyl or iso-propyl).

In various embodiments, —R^{N} may be —H.

In various embodiments, —R^{Z} may be —H. In other embodiments, —R^{Z} may be methyl, ethyl, propyl (n-propyl or iso-propyl) or butyl (n-butyl, iso-butyl, sec-butyl or tert-butyl), for example methyl or ethyl.

In various embodiments, —R^{O} may be —OH or —OR^{E}.

In various embodiments, —R^{E} may be methyl, ethyl, propyl (n-propyl or iso-propyl), butyl (n-butyl, iso-butyl, sec-butyl or tert-butyl), pentyl (n-pentyl, iso-pentyl or neo-pentyl) or hexyl, for example methyl, ethyl or propyl (n-propyl or iso-propyl).

Suitably, —R¹ and —R² (or —R² and —R³) may be joined together to form a 5- or 6-membered ring R^{D} optionally substituted with one or more hydroxyl or ═O groups and/or C₁₋₆ alkyl groups, in particular methyl groups, optionally with —R^{O} being —OH and/or —Z═ being —CH═.

In various embodiments, ring R^{D} may be a 6-membered ring, optionally substituted with one or more C₁₋₆ alkyl groups, for example methyl groups.

In an embodiment, the compound may have the structure:

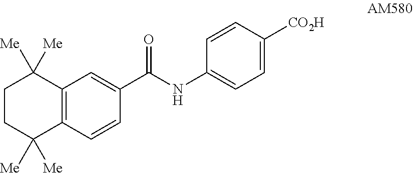 [SMILES: CC1(C)CCC(C)(C)c2cc(C(=O)Nc3ccc(C(=O)O)cc3)ccc21]

Or in an embodiment, the compound may have the structure:

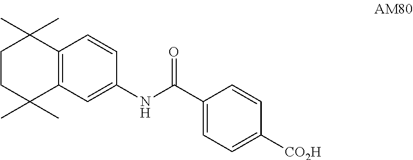 [SMILES: CC1(C)CCC(C)(C)c2cc(NC(=O)c3ccc(C(=O)O)cc3)ccc21]

In various embodiments, two of —R¹, —R² and —R³ (preferably —R² and —R³) are independently —O—R^{A} with the remaining —R¹, —R² or —R³ being —X, optionally with —R^{O} being —OH and/or —Z═ being —CR^{ZZ}═ with —R^{ZZ} being saturated aliphatic C₁₋₄alkyl, in particular methyl.

In an embodiment, the compound may have the structure:

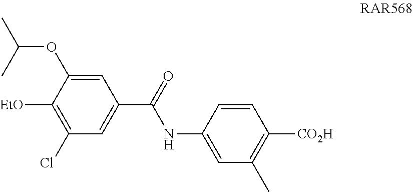 [SMILES: CCOc1c(Cl)cc(C(=O)Nc2ccc(C(=O)O)c(C)c2)cc1OC(C)C]

In some embodiments, the RARα agonist is selective for RARα over RARβ or RARγ and does not produce significant agonistic effects on RARβ or RAR γ. In some embodiments, the RARα agonist is selective for RARα over RARβ or RARγ and has a greater than 10-fold, 20-fold, 30-fold, 40-fold, 50-fold, 75-fold, 100-fold, 150-fold, 200-fold, 250-fold, 300-fold, 400-fold, 500-fold, 600-fold, 700-fold, 800-fold, 900-fold, 1000-fold, 1100-fold, 1200-fold, 1300-fold, 1400-fold, 1500-fold, 1600-fold, 1700-fold, 1800-fold, 1900-fold, 2000-fold or more selectivity for RARα over RARβ or RAR γ. In some instances, about 100% or at least about 99%, 95%, 90%, 85%, 80%, 75%, 70%, 65% or 60% of the effect of the agonist impacts RARα as compared to combined impact on RARβ or RAR γ.

Functional analogues of RARα agonists include agents that prevent the catabolism (or breakdown) of retinoids (for example retinoic acid), allowing the signal from retinoic acid itself to increase. Such agents may include retinoic acid metabolism blocking agents (RAMBAs), which are drugs that inhibit the catabolism of retinoids.

RAMBAs temporarily raise the endogenous levels of All Trans Retinoic Acid (ATRA) in vivo. In doing so, they induce a local retinoid effect and avoid excessive systemic retinoid exposure, thereby avoiding some of the toxicity issues associated with retinoic acid agonists. RAMBAs will act as RARα agonists. In some embodiments, RAMBAs include ketoconazol, liarozol, and/or tararozol.

Particularly suitable RARα agonists, analogues or derivatives thereof are those capable of inducing the expression of gut-homing molecules in Tregs. The gut-homing molecule is preferably integrin α4β7 and/or CCR9 and/or any other gut-homing molecules induced by RARα. The RARα agonist may suitably induce the expression of gut-homing molecules in Treg cells and increase trafficking of Tregs to the gut, gut tissue or gut cells. The increase in expression of gut-homing molecules and/or trafficking through the use of an RAR agonist selective for RARα over RARβ or RARγ may be at least 10%, 20%, 30%, 40%, 50%, 60%, 70%, 80%, 90%, 100% or more compared to the use of an RAR agonist selective for RARβ or RARγ or compared to using ATRA. Other qualities exhibited by suitable RARα agonists include reduced off target retinoid effects, reduced cytotoxicity, reduced genotoxicity, and a greater selectivity for RARα compared to RARβ and RAR γ.

RAR568 shows a selectivity profile against human RARs with an EC50 v of 0.59 nM/L and 290-fold greater selectivity for RARα over RARβ and >13,000 fold selectivity over RAR γ.

The at least one RARα agonist, for example RAR568, is added to the culture and/or expansion media preferably at a concentration of between 0.5 nM to 2 nM, suitably 1 nM and preferably maintained within said concentration range for the duration of the culturing step. The Tregs may be cultured in the culture/expansion media supplemented with at least one RARα agonist for up to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 days, suitably for five days.

The expansion is carried out to at least a 100-fold expansion, preferably to a greater than 1,000-fold. The expansion will depend upon the degree of stimulation and length of the culture.

As used herein “expanded” means that a cell or population of cells has been induced to proliferate. The expansion of a population of cells may be measured for example by counting the number of cells present in a population. The phenotype of the cells may be determined by methods known in the art such as flow cytometry.

The first aspect of the present invention therefore provides a method for making ex vivo expanded Tregs, comprising:
(i) Obtaining a Treg-containing biological sample from a subject having an immune-mediated gut disorder;
(ii) Isolating Tregs from the biological sample, using for example cell sorting;
(iii) Expanding the Tregs of step (ii) comprising contacting the Tregs with an effective amount of at least one RARα agonist and obtaining ex vivo expanded Tregs.

The ex vivo expanded Tregs obtained by the method according to the first aspect of the present invention may then be introduced into the same or different subject suffering from an immune-mediated gut disorder, optionally followed by the step of monitoring for or detecting a resulting improvement in the disorder in the subject.

The RARα agonist, functional analogue or derivative thereof is substantially removed prior to (re)infusion/(re)introduction into the subject. This typically occurs through the normal processing of the cells.

According to a second aspect of the present invention, there is provided ex vivo expanded Tregs having increased capacity for gut-homing and having previously been contacted with at least one RARα agonist, functional analogue or derivative thereof. The increased capacity for gut-homing may be due to changed expression, for example, increased expression of gut homing molecules such as α4β7 integrin and/or CCR9. Furthermore, ex vivo expanded Treg cells obtainable or obtained by the methods of the invention demonstrate superior gut homing both in vitro and in vivo. This has been shown by the inventors using a dynamic in vitro system as well as in a humanised xenograft mouse model of human intestinal xenografts. The Tregs may optionally be obtainable or obtained by the methods of the invention. The Tregs may exhibit increased capacity for gut-homing and/or changed, for example, increased expression of α4β7 integrin and/or CCR9 and/or improved Treg retention and/or increased potency.

The present invention demonstrates that Treg culture/expansion with the addition of an RARα agonist increases expression of gut homing molecules, particularly α4β7 integrin and/or CCR9. Other methods to increase expression of gut homing molecules, particularly α4β7 integrin and/or CCR9, include modifying Tregs to overexpress α4β7 integrin and/or CCR9 and/or other gut-homing molecules. In vivo approaches for replicating the effects of the present invention may include direct targeting of Tregs, for example, using nanoparticles or bispecific antibodies which selectively target Tregs (rather than Teffs) and which may be conjugated to a RARα agonist, functional analogue or derivative thereof. For example, an antibody to a Treg-specific target (such as LAG3, GITR, CTLA-4) could be conjugated to an RARα agonist, functional analogue or derivative thereof and given directly to a patient.

According to a third aspect of the present invention, there is provided modified Tregs which are modified to (over)express gut-homing molecules, particularly α4β7 integrin and/or CCR9. The sequences for these and other gut-homing molecules are known in the art and are readily available. For example, SEQ ID NO: 1 provides the nucleotide sequence for integrin alpha 4; SEQ ID NO: 2 provides the amino acid sequence for integrin alpha 4, isoform 1; SEQ ID NO: 3 provides the amino acid sequence for integrin alpha 4, isoform 2; SEQ ID NO: 4 provides the nucleotide sequence for integrin beta 7; SEQ ID NO: 5 provides the amino acid sequence for integrin beta 7, isoform 1; SEQ ID NO: 6 provides the amino acid sequence for integrin beta 7, isoform 2; SEQ ID NO: 7 provides the nucleotide sequence for CCR9; and SEQ ID NO: 8 provides the amino acid sequence for CCR9.

Integrin alpha-4 and integrin beta-7 can pair to form the heterodimer α4β7 integrin. The modified Tregs, modified to (over)express α4β7 integrin and/or CCR9, may (over)express any of the aforementioned amino acid sequences (or a combination thereof, for example in the case of expression of α4β7 integrin), or a sequence (or relevant combination of sequences for α4β7 integrin expression, for example) having at least 70%, 75%, 80%, 85%, 90%, 95% or more sequence identity to the aforementioned amino acid SEQ ID NOs.

The modified Tregs may (over)express α4β7 integrin and/or CCR9 encoded by a nucleotide sequence according to any of SEQ ID NOs 1, 4 and 7 (or a combination thereof, for example, in the case where the nucleotide sequence is encoding an α4β7 integrin), or a sequence having at least 70%, 75%, 80%, 85%, 90%, 95% or more sequence identity to the aforementioned nucleotide SEQ ID NOs (or a combination thereof in the case where the nucleotide sequence is encoding an α4β7 integrin, for example).

A “modified” Treg as used herein means a Treg which has been modified to comprise and overexpress at least one gut-homing molecule, which molecule(s) is/are introduced into the Treg and which are not naturally encoded in the unmodified Treg and/or which are in addition to the endogenous gut-homing genes.

Methods for genetically engineering or modifying cells are known in the art and include, but are not limited to, genetic modification of cells e.g. by transduction such as retroviral or lentiviral transduction, transfection (such as transient transfection—DNA or RNA based) including lipofection, polyethylene glycol, calcium phosphate and electroporation. Any suitable method may be used to introduce a gut-homing nucleic acid sequence into a Treg. The gut-homing nucleic acid may be represented by SEQ ID NOs 1, 4 and 7 (or a combination thereof, for example in the case where the nucleotide sequence encodes an α4β7 integrin), or may be represented by a sequence having at least 70%, 75%, 80%, 85%, 90%, 95% or more sequence identity to the aforementioned nucleotide SEQ ID NOs (or a combination thereof in the case where the nucleotide sequence is encoding an α4β7 integrin, for example).

Accordingly, there is provided a modified Treg that has been modified to comprise and to overexpress or express a gut-homing molecule, wherein said (over)expression is relative to a corresponding unmodified Treg. Modified Tregs of the present invention may be generated by introducing DNA or RNA coding for the gut-homing molecule, preferably α4β7 integrin, by one of many means including transduction with a viral vector, transfection with DNA or RNA. The modified Treg of the invention may be made by introducing to an unmodified Treg (e.g. by transduction or transfection) the polynucleotide or vector as defined herein. Suitably, the Treg to be modified may be from a sample isolated from a subject having an immune-mediated gut disorder.

Suitably, a modified Treg is a Treg having a genome modified e.g. by transduction or by transfection. Suitably, a modified Treg is a Treg whose genome has been modified by retroviral transduction. Suitably, a modified Treg is a Treg whose genome has been modified by lentiviral transduction.

As used herein, the term “introduced” refers to methods for inserting foreign DNA or RNA into a cell. As used herein the term introduced includes both transduction and transfection methods. Transfection is the process of introducing nucleic acids into a cell by non-viral methods. Transduction is the process of introducing foreign DNA or RNA into a cell via a viral vector. Modified Tregs according to the present invention may be generated by introducing DNA or RNA by one of many means including transduction with a viral vector, transfection with DNA or RNA.

Tregs may be activated and/or expanded prior to, or after, the introduction of a polynucleotide encoding the gut-homing molecule. In such cases where activation/expansion occurs after introduction of the gut-homing molecule into the Treg, the expansion/culture media may not need to be supplemented with an RARα agonist or at least not to the same levels.

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that the skilled person may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to those of skill in the art. They may also be cloned by standard techniques. Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

The present polynucleotide may further comprise a nucleic acid sequence encoding a selectable marker. Suitably selectable markers are well known in the art and include, but are not limited to, fluorescent proteins—such as GFP. The nucleic acid sequence encoding a selectable marker may be provided in combination with a nucleic acid sequence encoding the gut-homing molecule in the form of a nucleic acid construct. Such a nucleic acid construct may be provided in a vector.

The use of a selectable marker is advantageous as it allows Tregs in which a polynucleotide or vector of the present invention has been successfully introduced (such that the encoded gut-homing molecule is expressed) to be selected and isolated from a starting cell population using common methods, e.g. flow cytometry.

The polynucleotides used in the present invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

A vector is a tool that allows or facilitates the transfer of an entity from one environment to another. In accordance with the present invention, and by way of example, some vectors used in recombinant nucleic acid techniques allow entities, such as a segment of nucleic acid (e.g. a heterologous DNA segment, such as a heterologous cDNA segment), to be transferred into a target cell. Vectors may be non-viral or viral. Examples of vectors used in recombinant nucleic acid techniques include, but are not limited to, plasmids, mRNA molecules (e.g. in vitro transcribed mRNAs), chromosomes, artificial chromosomes and viruses. The vector may also be, for example, a naked nucleic acid (e.g. DNA). In its simplest form, the vector may itself be a nucleotide of interest.

The vectors used in the invention may be, for example, plasmid, mRNA or virus vectors and may include a promoter for the expression of a polynucleotide and optionally a regulator of the promoter.

Vectors comprising polynucleotides of the invention may be introduced into cells using a variety of techniques known in the art, such as transformation and transduction. Several techniques are known in the art, for example infection with recombinant viral vectors, such as retroviral, lentiviral, adenoviral, adeno-associated viral, baculoviral and herpes simplex viral vectors; direct injection of nucleic acids and biolistic transformation.

Non-viral delivery systems include but are not limited to DNA transfection methods. Here, transfection includes a process using a non-viral vector to deliver a gene to a target cell.

Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nat. Biotechnol. (1996) 14: 556) and combinations thereof.

Other methods for modifying Tregs to overexpress the gut-homing molecule include gene editing approaches (such as CRISPR). Various methods are known in the art for editing nucleic acid, for example to cause a gene knockout or expression of a gene to be downregulated. For example, various nuclease systems, such as zinc finger nucleases (ZFN), transcription activator-like effector nucleases (TALEN), meganucleases, or combinations thereof are known in the art to be used to edit nucleic acid and may be used in the present invention. In recent times, the clustered regularly interspersed short palindromic repeats (CRISPR)/CRISPR-associated (Cas) (CRISPR/Cas) nuclease system has become more commonly used for genome engineering. The CRISPR/Cas system is detailed in, for example WO2013/176772, WO2014/093635 and WO2014/089290. Its use in T-cells is suggested in WO2014/191518.

The time-limiting factor for generation of mutant (knock-out, knock-in, or gene replaced) cell lines was the clone screening and selection before development of the CRISPR/Cas9 platform. The term “CRISPR/Cas9 platform” as used herein, refers to a genetic engineering tool that includes a guide RNA (gRNA) sequence with a binding site for Cas9 and a targeting sequence specific for the area to be modified. The Cas9 binds the gRNA to form a ribonucleoprotein that binds and cleaves the target area. Before CRISPR/Cas9, mammalian genome editing could be multiplexed, but selection for particular mutations, transgene insertions, or gene deletions required antibiotic resistance markers or laborious PCR based screening methods.

In addition to the CRISPR/Cas 9 platform (which is a type II CRISPR/Cas system), alternative systems exist including type I CRISPR/Cas systems, type III CRISPR/Cas systems, and type V CRISPR/Cas systems. Various CRISPR/Cas9 systems have been disclosed, including Streptococcus pyogenes Cas9 (SpCas9), Streptococcus thermophilus Cas9 (StCas9), Campylobacter jejuni Cas9 (CjCas9) and Neisseria cinerea Cas9 (NcCas9) to name a few. Alternatives to the Cas system include the Francisella novicida Cpf1 (FnCpf1), Acidaminococcus sp. Cpf1 (AsCpf1), and Lachnospiraceae bacterium ND2006 Cpf1 (LbCpf1) systems. Any of the above CRISPR systems may be used in methods of the invention to generate modified Tregs.

Target genes may be edited, for example using the above methods, by deleting, inserting or substituting one or more nucleotides within said target gene, leading to the knockout of that gene, or the downregulation of expression of that gene.

The modified Tregs of the present invention advantageously have improved functionality which may be manifested by improved trafficking of Tregs to the gut of a mammal and/or improved Treg retention and/or increased potency. a4b7 is also a retention signal for T cells in the gut, advantageously leading not only to increased trafficking but also to increased retention. Increased potency may result from the appropriate localisation of Tregs within the inflamed mucosa, for example.

According to a fourth aspect of the present invention, there is provided a pharmaceutical composition comprising modified and/or ex vivo expanded Tregs for the treatment, amelioration or prevention of an immune-mediated gut disorder, the disorder being as defined herein.

A pharmaceutical composition is a composition that comprises or consists of a therapeutically effective amount of a pharmaceutically active agent, the pharmaceutically active agent here being modified and/or ex vivo expanded Tregs. It preferably includes a pharmaceutically acceptable carrier, diluent or excipient (including combinations thereof).

Acceptable carriers or diluents for therapeutic use are well known, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s) or solubilising agent(s).

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

According to a fifth aspect of the present invention, there is provided a method of treating an immune-mediated gut disorder, comprising contacting Tregs previously obtained from a subject with an immune-mediated gut disorder with at least one RARα agonist before reintroducing the treated Tregs into the same or different subject in need of treatment or relief from an immune-mediated gut disorder.

The method of treatment may treat the immune-mediated gut disorder, or may ameliorate the symptoms thereof, or may in some cases prevent the immune-mediated gut disorder. The immune-mediated gut disorder may be inflammatory bowel disease (IBD), particularly Chron's Disease (CD) and/or ulcerative colitis (UC). The immune-mediated gut disorder may be colitis (such as checkpoint-related colitis (colitis associated with the treatment for solid cancers treated with checkpoint inhibitors (such as anti-CTLA4 and/or anti-PD1/PDL1/L)), treatment-resistant Clostridium difficile-associated colitis etc.), GvHD, where the gut is involved.

Treg cells before ex vivo treatment exhibit a higher proportion of α4β7+Teff in, for example subjects with CD, whereas in healthy controls there is a substantially more equal balance between α4β7+ Treg and Teff. The present invention therefore aims to restore the balance to more equal levels of α4β7+ Treg and Teff.

A method for treating a disease also relates to the therapeutic use of the Tregs of the present invention, both ex vivo expanded Tregs and modified Tregs. In this respect, the cells may be administered to a subject having an immune-mediated gut disorder, in order to lessen, reduce or improve at least one symptom associated with the disorder and/or to slow down, reduce or block the progression of the condition.

The method for preventing a disease relates to the prophylactic use of ex vivo expanded Tregs or modified Tregs of the present invention. In this respect, the Tregs may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease. Such prophylactic use may be particularly suited to prevent colitis (such as checkpoint-related colitis (colitis associated with the treatment for solid cancers treated with checkpoint inhibitors (such as anti-CTLA4 and/or anti-PD1/PDL1/L)), treatment-resistant Clostridium difficile-associated colitis etc.), GvHD, where the gut is involved.

Suitably, the therapeutic methods of the invention may comprise the step of administering ex vivo expanded Tregs and/or modified Tregs and/or a pharmaceutical composition of the present invention, or obtainable (e.g. obtained) by a method according to the present invention, or a polynucleotide or a vector comprising and capable of (over)expressing a gut-homing molecule (for example in a pharmaceutical composition as described above) to a subject.

According to a sixth aspect of the present invention, there is provided ex vivo expanded Tregs, modified Tregs a pharmaceutical composition, RARα agonists and analogues and derivates thereof, all according to the present invention, for use in the treatment, amelioration or prevention of an immune-mediated gut disorder, as defined herein.

The present invention also provides use of ex vivo expanded Tregs, modified Tregs a pharmaceutical composition, RARα agonists and analogues and derivates thereof according to the present invention in the manufacture of a medicament for the treatment, amelioration or prevention of an immune-mediated gut disorder, as defined herein.

According to a seventh aspect of the present invention, there is provided culture and/or expansion media for use in the production of ex vivo expanded Tregs, which media comprise at least one RARα agonist or a functional analogue or derivative thereof. The RARα agonist or a functional analogue or derivative thereof are as defined herein.

Throughout the description and claims of this specification, the words “comprise” and “contain” and variations of the words, for example “comprising” and “comprises”, mean “including but not limited to”, and do not exclude other components, integers or steps.

Moreover, the singular encompasses the plural unless the context otherwise requires: in particular, where the indefinite article is used, the specification is to be understood as contemplating plurality as well as singularity, unless the context requires otherwise. Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 shows expression of gut homing molecules in CD. (a) Gating strategy to define Treg and Teff population and their expression of integrin β7 (b) Differential expression of integrin β7 in peripheral blood and colon of CD patients. Wilcoxon matched pairs signed rank test was used to determine statistical significance in all matched values. ***p<0.001, **p<0.005, *p<0.05, ns=p>0.05 were used throughout. (N=63 CD Peripheral Blood, N=20 CD colon) (b) Representative flow plots of integrin β7 MFI and (α4β7+ expression in CD patients compared to HC (c) Patients with Crohn's Disease have significantly less α4β7 positive Treg in circulation compared to healthy controls (p=0.006). Mann Whitney Test with a two tailed p value was used to determine significance in all unmatched values. (N=56 CD Peripheral blood, N=41 HC Peripheral blood, N=24 active CD) (d) There is a higher proportion of α4β7⁺ Teffs than Tregs in the lamina propria of Crohn's disease patients (p=0.001). There is no difference in proportions of α4β7⁺ Tregs to Teffs in healthy controls (N=15 CD colon, N=16 HC colon) (e) Patients with active Crohn's Disease have significantly more circulating Tregs than healthy controls (p=0.04). There is a reduced proportion of circulating Teff in CD vs HC (p=0.01 HC vs Active CD, p=0.03 HC vs Inactive CD). (N=64 CD, N=41 HC) (f) The colonic homing marker GPR15 is expressed on a greater proportion of Teff in CD compared to HC (p=0.04). (N=64 CD, N=41 HC) (g) Higher proportion of Treg and Teff express the small bowel homing molecule CCR9 (p=0.03 Treg, p=0.0004 Teff).(N=43 CD, N=37 HC). (h) Higher proportion Treg than Teff in CD colon express GPR 15 (p=0.0039, N=19). When compared to HC CD Teff express more GPR 15 (p=0.02, N=19 CD, N=22 HC).

FIG. 2 shows that RARα is more efficient at inducing α4β7 during in vitro culture (a) Gating strategy to define α4β7 expression on CD25^{high}CD127^{low}CD45RA⁺Tregs freshly isolated and following expansion (b) Cumulative data demonstrating significant and consistent induction of α4β7 in cultures treated with RAR568 (c) Dose response curve demonstrating greater efficacy of RAR568 at inducing expression of integrin β7 (d) FOXP3 expression is unchanged in Tregs expanded in the presence of retinoids compared to standard conditions. (e) and (f) shows in-vitro treatment with retinoids maintains suppressive ability and phenotypic stability. (e) Suppression assay comparing cells expanded in the presence of ATRA or RAR568 (f) Stability assay demonstrating that cells treated with retinoids maintain their phenotype under pro-inflammatory conditions.

FIG. 3 shows treatment with RAR568 reduces off target retinoid effects. Genes upregulated with fold increase, with p≤0.05. Cells treated with RAR568 or ATRA compared to those treated with Rapamycin only. Gene expression compared against a published list of RARγ target genes. (a) Volcano plot demonstrating increased expression of genes associated with pro-inflammatory T cell lineage in cells treated with ATRA (top panel) and more specific upregulation of α4 in cells treated with RAR568. (b) Increased expression of RARγ target genes in cells treated with ATRA.

FIG. 4 shows induction of α4β7 is functionally relevant in vitro. (a) In-vitro trafficking assay demonstrating a significant improvement in RAR568 treated Treg crawling, rolling and adhesion when exposed to the α4β7 ligand MadCAM when compared to Rapa only treated cells. (b) Cumulative data from N=3 trafficking assays.

FIG. 5 shows induction of α4β7 is functionally relevant in vivo (a) Experimental design: C.B17 SCID mice transplanted with human foetal small bowel that has matured over 12-16 weeks, have inflammation induced with Mycobacterium Avium Paratuberculosis (MAP) in the xenografts at day −3 prior to Treg transfer. Mice are injected with anti-asialo GM1 antibody at day −2 prior to transfer in order to deplete natural killer (NK) cells. On the day of Treg transfer, mice were treated with Tregs that were either expanded with Rapamycin alone or with the addition of RAR568. Mice also received 1000IU of rhlL-2 IP on the day of Treg transfer, to support the Treg in circulation. After three days in circulation, the presence of CFSE labelled Tregs was assessed by FACS in digested xenograft samples and immunofluoresence on frozen sections. (b) Representative FACS plots from Treg transfer into SCID mouse xenografted with human foetal small bowel, demonstrating the presence of CFSE labeled human Tregs in xenografts after transfer of either Rapa or Rapa+RAR568 treated cells. (c) Cumulative data from two independent experiments, N=5 Rapa, N=6 Rapa+RAR568 (d) Cumulative data demonstrating increased trafficking of RAR568 treated cells to inflamed xenografts. Induction of α4β7 is functionally relevant in vivo (e) Control XG, no Tregs, (f) XG from mouse treated with Rapa Tregs (g) XG from mouse treated with Rapa+RAR568 Tregs.

FIG. 6 shows the comparable effects of the induction of integrin α4β7 on Treg surface by RARα agonists AM80, AM580 and RAR568. Bulk Tregs (CD4+CD25+CD127−) Tregs (50,000 per well) were expanded in vitro with reducing concentrations of the agonists. Culture conditions: 2 aCD3/aCD28 beads/cell, 1000IU/mL IL-2, 0.1 nM Rapamycin+Agonist in X-vivo 15. Following 12 days stimulation, cells were stained for CD4, CD25, CD127, FOXP3, Integrin a4, Integrin b7, CD15s, CD161 and acquired on a BD symphony flow cytometer. Data was analysed in Flowjo and Prism.

FIG. 7 shows Gating strategy for α4β7 expression and β7 MFI for CD and HC samples.

FIG. 8 shows the effect of CD disease activity, thiopurines and biologics on expression of gut homing molecules, CCR9 expression in peripheral blood.

FIG. 9 shows that high expression of CD62L is maintained following expansion and is not affected by RAR568 treatment.

FIG. 10 shows the experimental set up for in-vitro trafficking experiments using MAdCAM-1 coated ibidi flow chamber.

FIG. 11 shows representative plots from spleens of mice treated with either Rapa or Rapa+RAR568 Tregs.

### EXAMPLES

The present invention will now be described with reference to the following examples.

Materials and Methods

Patient Samples

CD PBMCs and tissue samples were obtained from patients attending endoscopy and outpatients at Guy's and St Thomas' NHS Trust. Ethics approval for human blood and tissue collection was obtained from NRES Committee—London Riverside (REC reference: 15/LO/0151) and Guy's and St Thomas' NHS Trust R&D (R&D REF: R1115/N122)

Cell Culture Media and Buffers

“Complete X-VIVO-15” (Lonza, Walkersville, MD) was used for ex vivo Treg expansion, Treg cytokine challenge experiments and Treg suppression assays. This was supplemented with 100 nM or 10 nM Rapamycin and all-trans retinoic acid (ATRA) 2 μM or 1 nM, or Rapamycin and RAR568 1 nM.

Other experiments were performed in RPMI 1640 medium (PAA Laboratories, Pasching, Austria) supplemented with HEPES (10 mM, Thermo Fisher Scientific, Loughborough, UK), L-glutamine (2 mM), penicillin (100IU/ml), streptomycin (100 g/ml), sodium pyruvate (1 mM), MEM nonessential amino acids (0.1 mM), and10% foetal calf serum (all PAA).

CD4 Isolation and Cell Sorting

Peripheral blood mononuclear cells (PBMCs) were isolated via Ficoll-Paque. CD4+ cells were enriched by MACS enrichment as per manufacturer's instructions. CD4+ cells were FACS sorted (BD FACSAria; BD Biosciences, Franklin Lakes, N.J.) into CD4^{,}CD25^{h}′g^{h}CD127¹° wCD45RA, and effector T cell (CD4+CD25-) populations.

LPMC Isolation

Colonic biopsies collected from CD patients and HC were washed in Hank's Balanced Salt Solution (HBSS) containing 1 mM EDTA. Samples were then digested using Collagenase Ia (Sigma) 1mg/ml and DNAse I (Roche) 1 μl/ml. Following digestion, cells were passed through a 100 μm cell filter and counted.

In vitro Treg Expansion

FACS-sorted Treg populations were plated at 1×10⁶ or 0.5×10⁶ in X-VIVO-15 medium and activated with anti-CD3/anti-CD28 coated beads (Dynabeads®, Invitrogen, Paisley, UK) at 1:1 bead: cell ratio. Rapamycin was added at day 0 of culture at a final concentration of 100 nM/L +/−ATRA 1 nM/L or RAR568 1 nM/L. Next, 1,000 IU/ml recombinant human IL-2 (rhlL-2) (Proleukin®, Novartis, Camberley, UK) was added at day 5 of culture. Cells were re-stimulated every 10-12 days and expanded for a total of 24-30 days. The phenotype and suppressive ability were assessed at the end of the culture period.

Assessment of Treg Suppressive Ability

Effector T cells (Teff) were labelled with carboxyfluorescein succinimidyl ester (CFSE, Invitrogen) according to standard protocols. Cells were washed with phosphate buffered saline (PBS) to remove excess protein. Cells were then incubated with a 1 μM/L CFSE solution in the dark at room temperature for 4 minutes. The reaction was then quenched with 9 ml of complete medium.

Teff were activated with anti-CD3/anti-CD28 micro beads at a bead: Teff ratio of 0.02:1. 1×10⁵ Teff were then cultured either alone or with Tregs in serial dilutions. The ratios of Teff: Treg were 1:1, 2:1, 4:1, and 8:1. This was done in X-VIVO-15 and proliferation rates were assessed by flow cytometry after 5 days of incubation.

Percentage suppression (S) of proliferation was calculated using the following formula:

S=100−[(c/d)×100]

Where c=percentage of proliferating precursors in the presence of Tregs and d=percentage of proliferating precursors in the absence of Tregs.

Flow Cytometry Analysis of Tregs

Flow cytometry panels have been designed to assess the subtypes of regulatory T cells in patients with CD as well as their expression of gut homing molecules. Gating was performed based on natural populations when assessing for CD4⁺CD25^{high}CD127_{low} populations, as well as for CD45RA⁺ Treg populations. Additionally, to minimize bias in the assessment of expression of gut homing markers and transcription factors, a fluorochrome minus one (FMO) panel was added for each marker of interest in each experiment that was performed.

Ibidi Flow Chamber Experiments

Ibidi (Martinsreid, Germany) μ-Slides VI″ were coated with recombinant human MAdCAM-1 (R&D Systems, Minneapolis, Minn.), at a concentration of 10μg/ml and incubated overnight. Cells from two CD patients that had previously undergone ex vivo expansion under two parallel conditions (Rapa and Rapa+RAR568) and had been frozen in liquid nitrogen were defrosted and rested overnight. Rested cells were then activated with rhCCL25 (R&D systems) and passed through the coated Ibidi flow chamber at a rate of 1 dyne/cm². The total number of cells, as well as those rolling, adherent and crawling was quantified from six randomly selected fields of view per treatment.

Estimation of Cytokine Concentrations

Cytokine concentrations were measured using the Ready-SET-Go sandwich ELISA kits from eBioscience.

Assessment of IL-17 and IFNγ Production Under Pro-Inflammatory Conditions

Ex vivo expanded Tregs were activated with CD3/CD28 beads at a 1:20 ratio and cultured at 10⁶cells/m1 in X-VIVO for 5 days at 37° C./5% CO₂, supplemented with the following cytokine cocktail: A) IL-2 (10 IU/mL, Proleukin); (B) IL-2, IL-1 (10 ng/mL), IL-6 (4 ng/mL) and transforming growth factor-β (TGF-β (5 ng/mL), IL-21 (25 ng/mL), IL-23 (25 ng/mL) (all R&D Systems). Supernatant IL-17 and interferon gamma (IFNγ) concentrations were measured by ELISA.

C.B-17 SCID Mouse Human Intestinal Xenotransplant Model

The C.B-17 SCID mouse human intestinal xenograft model has been previously described^{28,29}. Institutional Review Board (IRB) and Institutional Animal Care and Use Committee (IACUC) approvals were obtained prospectively (Ethics Committee for Animal Experimentation, Hebrew University of Jerusalem; MD-11-12692-4 and the Helsinki Committee of the Hadassah University Hospital; 81-23/04/04). Tregs were labelled with CFSE (Invitrogen) prior to transfer, as per manufacturer's instructions. Xenografts were processed as per LPMC digestion protocol. CFSE positive cells were detected by flow cytometry. Additionally, CFSE positive cells were detected by immunofluorescence on frozen sections from treated xenografts.

Immunoflouorescent Staining

Fresh xenograft sections were fixed and stored in OCT. Fixed cryostat sections were blocked with 20% fetal calf serum (FCS) and stained with rat anti human CD45 (Invitrogen) and mouse antihuman FOXP3 (Biolegend), followed by donkey anti-rat AF594 (Invitrogen) and donkey anti-mouse NL637 (RnD Systems). Negative controls were obtained from sections from xenografts that did not receive Treg transfer.

Statistical Analysis

Flow cytometric data were analysed with FlowJo 10.4.2 for MacOsX. Statistical analysis was performed with GraphPad Prism 6.0h for MacOsX. Continuous data are presented as mean±standard deviation for continuous (approximately) symmetrically distributed variables; as medians and interquartile ranges for skewed variables. Comparison of means and/or medians were performed using paired parametric and nonparametric tests as appropriate (paired t test or Wilcoxon signed rank test, respectively). For comparison of matched values (such as Treg and Teff in the same patient) the Wilcoxon matched pairs signed rank test was used. Mann Whitney Test with a two tailed p value was used to determine significance level in all unmatched values (such as comparisons between CD and HC). The CD and HC groups were broadly matched by age and gender. A p value of less than 0.05 was considered statistically significant throughout.

Gene array analyses were carried out using Partek® software with a 1-way ANOVA to assess for differential gene expression.

RNA Extraction and Gene Arrays

1) RNA extraction was performed using Qiagen RNEasy mini/micro kits as per manufacturer's instructions. The samples were checked for RNA quality using the Agilent 2100 Bioanalyzer and quantified using the Nanodrop (ND-1000 Spectrophotometer). Samples which passed QC (RIN>8) were chosen such that input amount of each sample was 3 ng.

2) SPIA cDNA was generated using the “Ovation Pico WTA System V2” kit from Nugen, following the manufacturer's instructions.

3) The SPIA cDNA was subjected to a QC check to assess quality (Agilent 2100 Bioanalyzer) and quantity (Nanodrop ND-1000 Spectrophotometer) for the next stage.

4) The SPIA cDNA was fragmented and Biotin-labelled using the “Encore Biotin Module” from Nugen according to the manufacturer's instructions and passed through QC checks to assess fragmentation size (Agilent 2100 Bioanalyzer).

6) Hybridization cocktails were prepared of the fragmented labelled-cDNA according to Nugen's recommendations and hybridized at 45° C. overnight in an oven.

7) The arrays were washed and stained using wash protocol FS450_0002 (Affymetrix protocol recommended for Human Gene 2.0 Arrays on the GeneChip Fluidics station 450.

8) The arrays were scanned using the Affymetrix GeneChip Scanner.

Results

Patients with Crohn's Disease (CD) have a Lower Proportion of Tregs Licensed to Traffic to the Gut than Teffs.

Peripheral blood samples were taken from 64 CD patients attending outpatient clinics, the IBD infusion unit or endoscopy at Guy's and St Thomas' NHS Trust and 41 healthy controls (HC) (patients attending outpatients for the management of irritable bowel syndrome (IBS), or undergoing colonoscopy for polyp surveillance/positive fecal occult blood test). Table 1 below outlines patient demographics. HC were matched for age and sex.

TABLE 1
Demographics of CD patients and HC included in the study

| Crohn's Disease Patients | | |
|---|---|---|
| Age (mean) | 40.35 | (±11.55) |
| Female sex (%) | 28 | (44) |
| Disease Distribution | | |
| L1 (%) | 12 | (19) |
| L2 (%) | 10 | (16) |
| L3 (%) | 40 | (65) |
| Medical Therapy | | |
| Biologic (%) | 31 | (50) |
| Thiopurine (%) | 35 | (56) |
| Vedolizumab (%) | 3 | (5) |
| Disease Activity | | |
| Active Disease (%) | 26 | (42) |
| Evidence of Mucosal Inflammation (%) | 23 | (37) |
| Healthy Controls (HCs) | | |
| Age (mean) | 45 | (±12.23) |
| Female sex (%) | 27 | (57) |

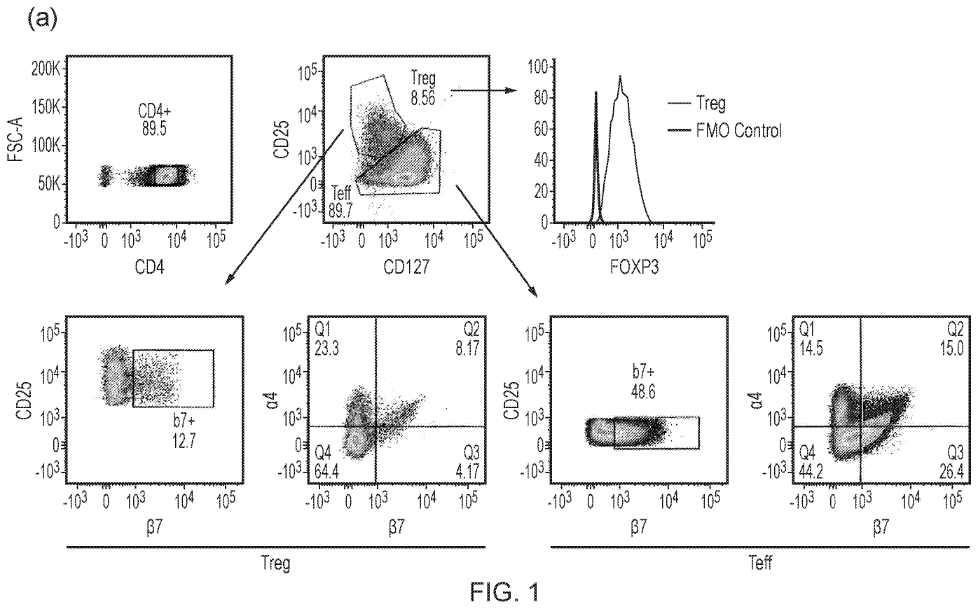

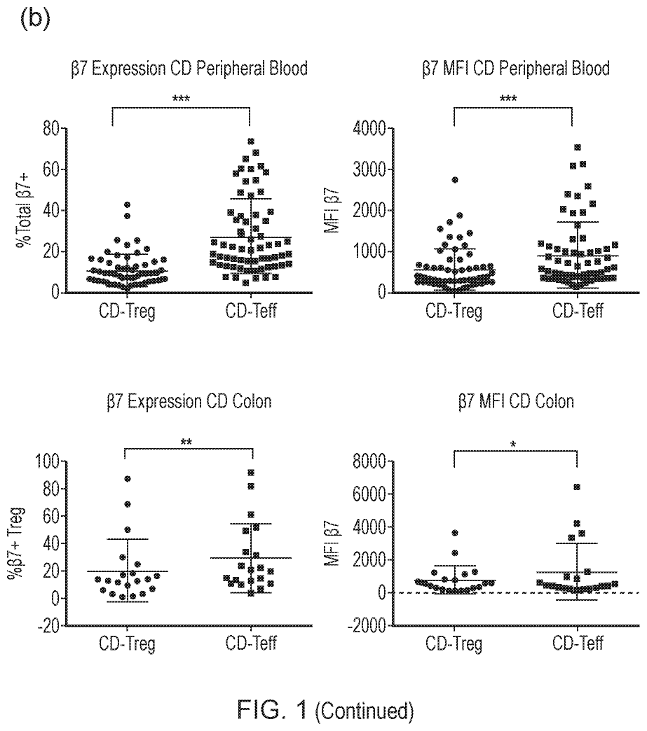

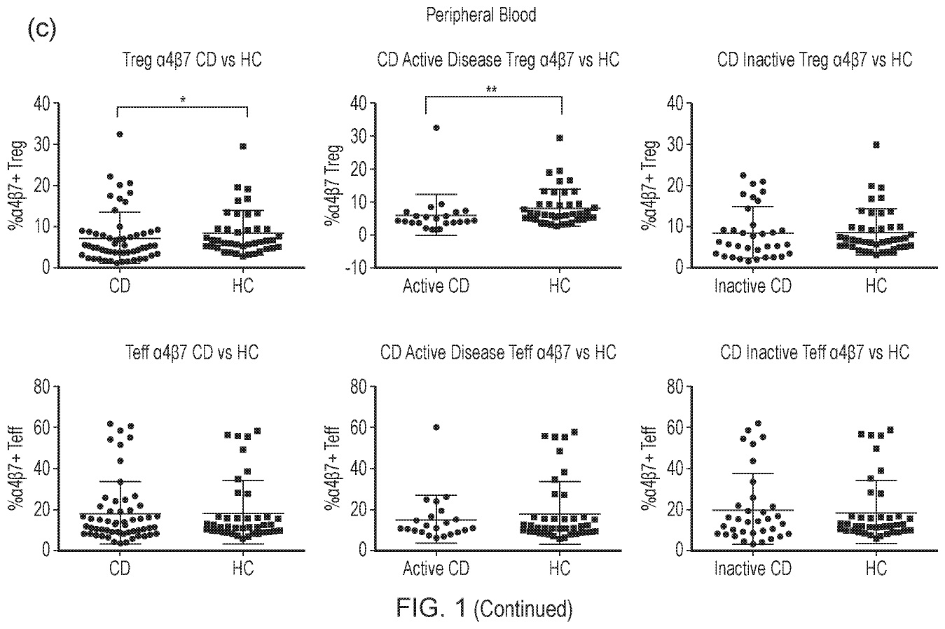

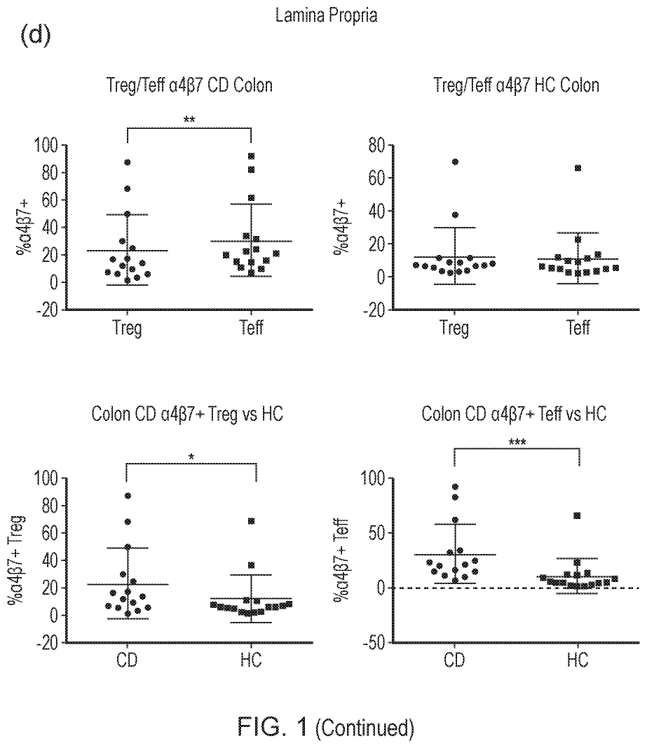

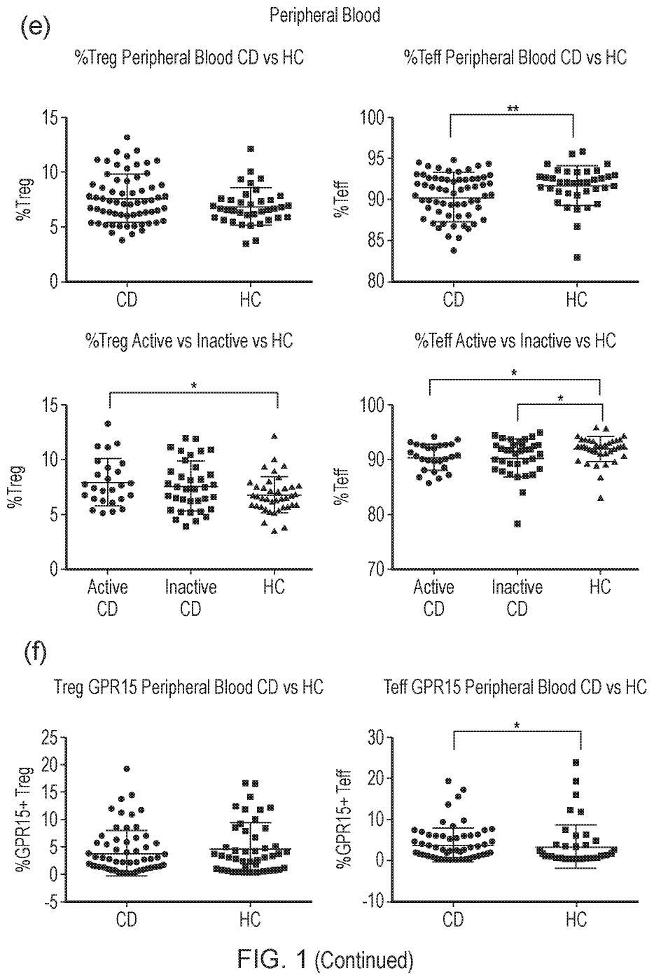

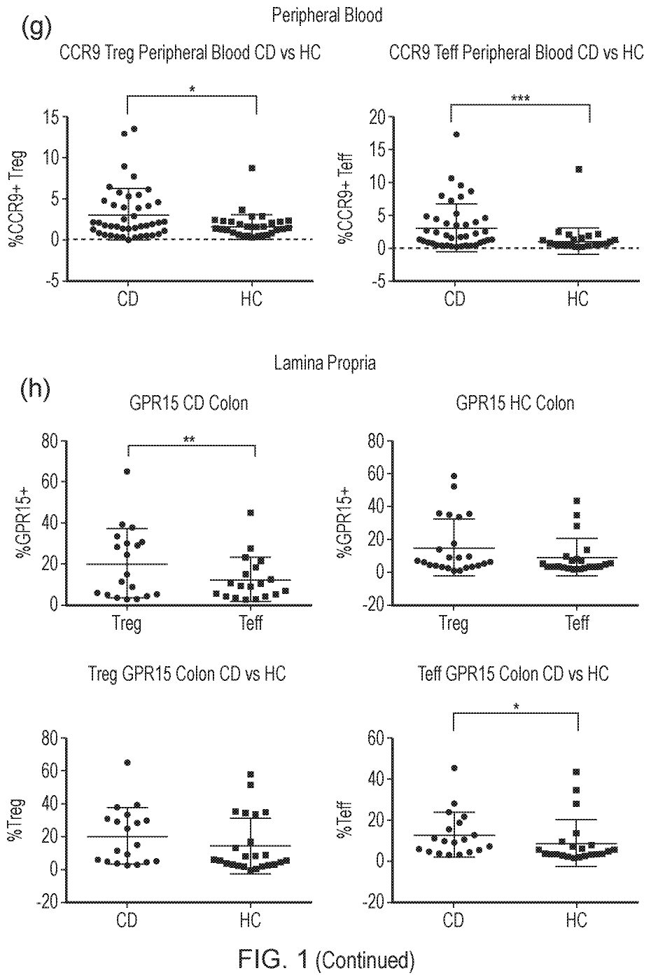

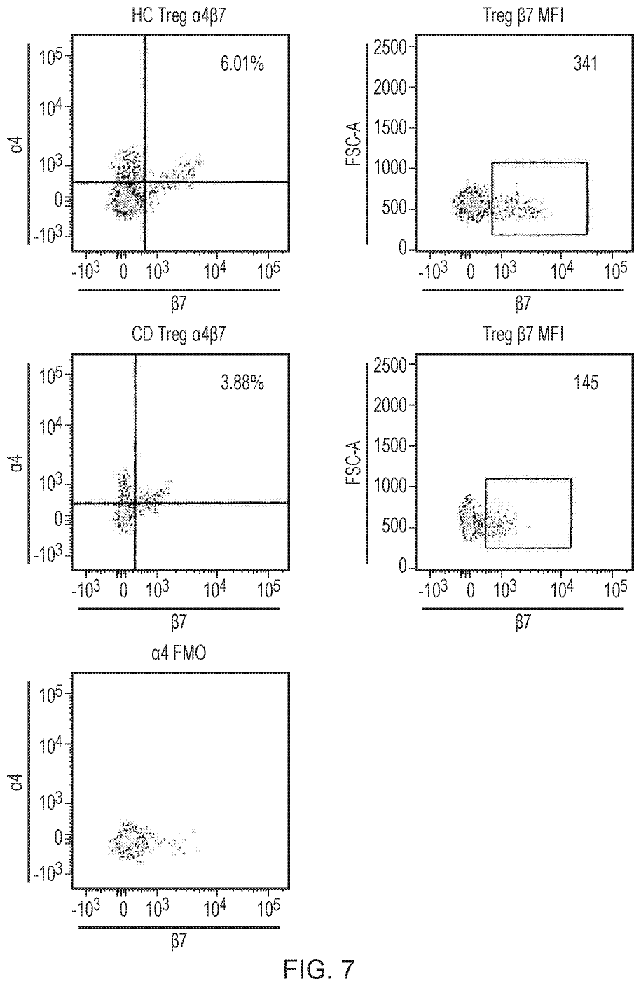

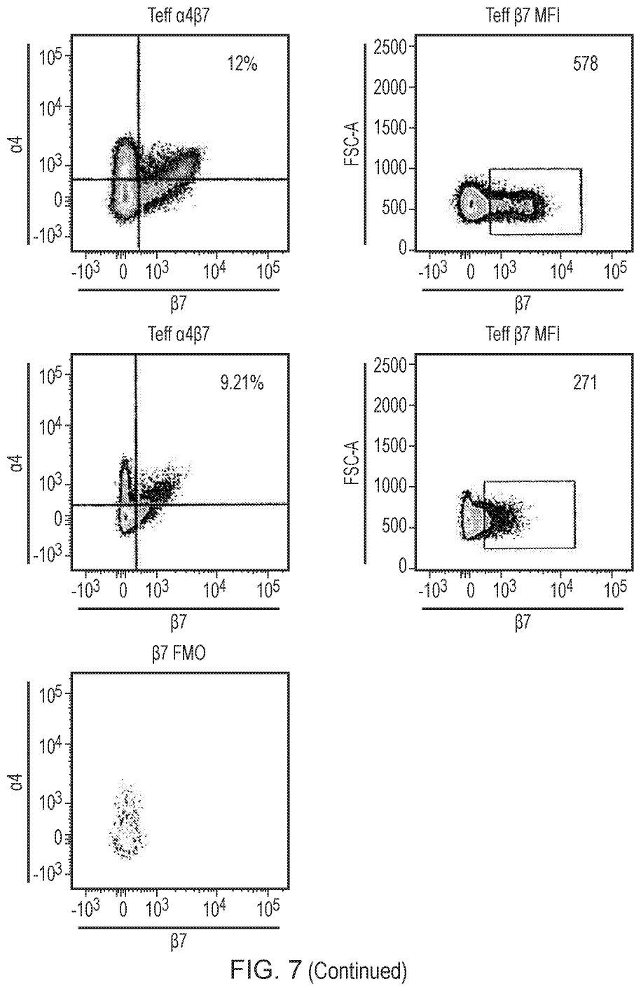

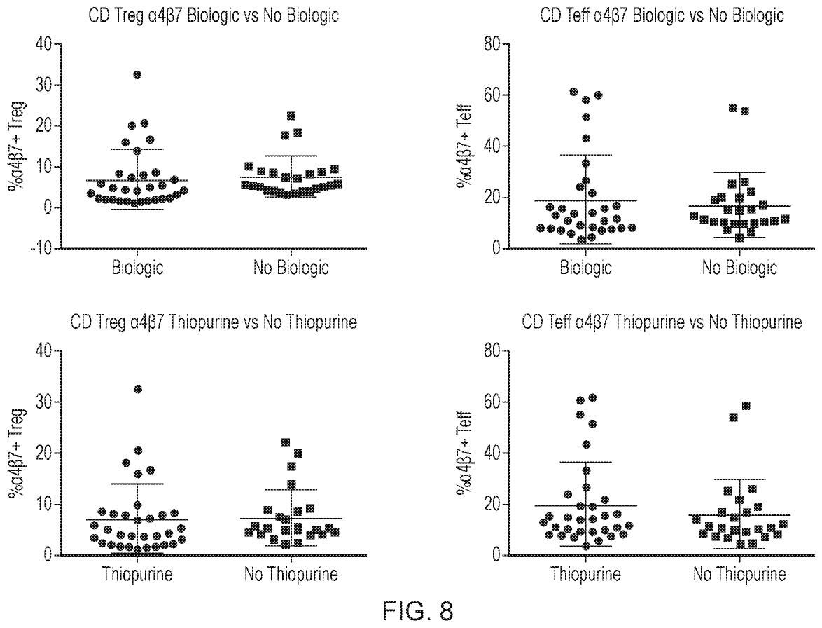

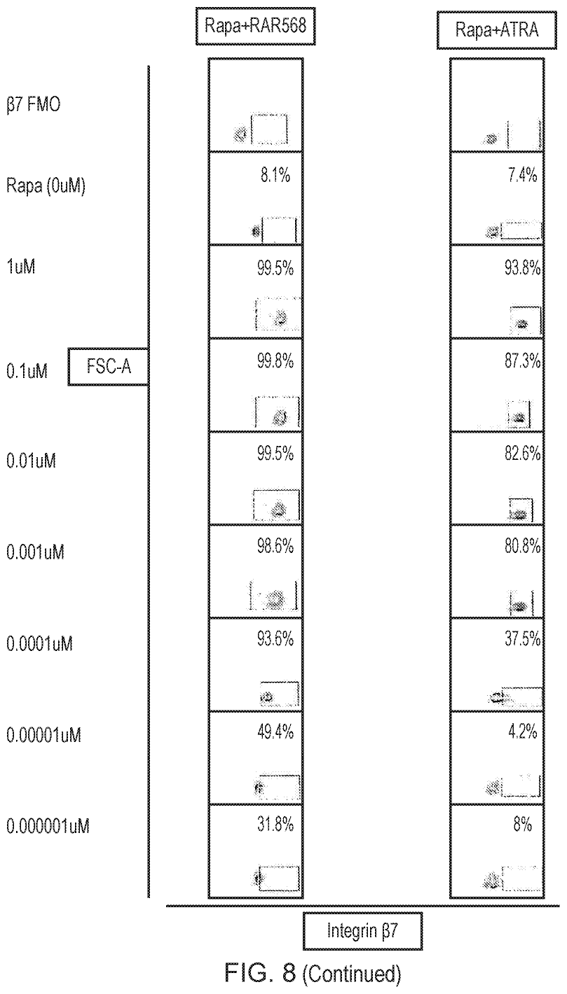

Colonic biopsies were also obtained from 19 CD patients and 22 HCs. PBMCs and LPMCs were isolated using standard Ficoll density gradient and DNAse/collagenase digestion protocols respectively. Tregs were identified as CD4⁺CD25^{hi}CD127^{lo}FOXP3⁺. Teff were identified as the CD4⁺CD25⁻CD127⁺FOXP3⁻population (gating strategy is shown in FIG. 1a). Significantly more Teff in the peripheral blood expressed integrin β7 compared to Tregs (27.91±18.19 vs 10.81±7.919, p<0.0001). In addition to a reduced percentage of cells expressing β7, there was also a difference in expression per cell, as assessed by the mean fluorescence intensity (MFI) of β7 (932±800.7 vs 575.4±509.4, p<0.0001) (Representative Flow plots FIG. 1a, Summary data FIG. 1b). Similarly, there was a significantly higher proportion of β7 positive Teff than Treg in the lamina propria of CD patients (30.94±26.4 vs. 23.75±25.56, p=0.0004). This difference was again associated with a reduced expression per cell of β7 on Tregs as assessed by MFI (p<0.05) (FIG. 1b). When compared to HC, there was a lower proportion of α4β7 positive Tregs in the circulation of patients with CD compared to HCs, representative flow plots of α4β7 gating in FIG. 7, summary data FIG. 1c (5.26 [3.61-8.73] vs 6.75 [5.25-9.65], p<0.05). This difference was even more profound when we compared CD patients with active disease only vs HCs (4.51 [3.8-7.05] vs. 6.71 [5.1-9.65], p=0.0063) (FIG. 1c). The proportion of α4β7⁺ circulating Tregs was not affected by thiopurine or biologic treatment (FIG. 8). Given the efficacy of the anti-α4β7 monoclonal antibody Vedolizumab in CD, we sought to examine the balance between regulatory and effector T cells in the lamina propria of CD patients and to compare this with HCs. There was a significantly higher proportion of α4β7⁺ Teffs compared with Tregs in the lamina propria of patients with CD (30.94±26.40 vs. 23.75±25.56, p=0.0016) (FIG. 1d). No such difference existed in HCs (FIG. 1d). When compared to HC, the lamina propria of CD patients had a significantly increased proportion of α4β7⁺ Tregs (14.2 [6.29-30] vs 6.38 [3.62-10.32], p=0.049). However, there was an even greater increase in the proportion of α4β7⁺ Teffs in CD compared to HCs (21.30 [14.7-34.1] vs 5.05 [2.76-10.8], p=0.0002) (FIG. 1d), suggesting an impaired balance of Teffs to Tregs in diseased tissue.

To ascertain whether the reduction in α4β7⁺ circulating Tregs was an isolated impairment or the result of a global Treg deficiency, we analyzed the proportion of circulating Tregs in patients with CD and compared it with that in HCs. We found that there was no difference in the percentage of circulating Tregs between CD patients and HC (7.24 [6.00-9.07] vs 6.52 [5.65-7.43], p =ns). Patients with CD however did have a significantly lower proportion of circulating Teffs than HCs (90.95 [88.20-92.58] vs 92.2 [91.00-93.5], p<0.05). When CD patients were separated based on disease activity, those with active disease had a significantly higher proportion of circulating Tregs compared to HCs (7.43 [6.26-9.25] vs 6.52 [5.65-7.44], p<0.05) (FIG. 1e). Thus we conclude that the decrease in α4β7⁺circulating Tregs is not due to a global Treg deficiency in CD. These findings are contrary to previous reports that there is an overall increase in the proportion of circulating Tregs in CD, which contracts during periods of disease activity but still remains higher than the proportion of circulating Tregs in HCs^{19, 30, 31}.

In order to assess whether the defect was specific for α4β7 expression or extended to other major gut trafficking molecules, we assessed the expression of the intestinal homing chemokine receptors GPR15 and CCR9 on Tregs and Teffs of patients with CD and compared these to HCs. There was no difference between the proportion of GPR15+ Tregs in circulation between patients with CD and HCs. However, there was a significantly higher proportion of GPR15⁺ circulating Teff in patients with CD (2.11 [0.86-5.93] vs 1.06 [0.43-3.45], p<0.05) (FIG. lf). On assessment of CCR9 expression, we found that significantly more Tregs (1.82 [0.73-4.5] vs 1.23 [0.67-2.09] p<0.05) and Teffs (1.55 [0.43-17.3] vs 0.49 [0.28-1.24] p=0.0004) expressed CCR9 in patients with CD compared with HCs (FIG. 1g). We then assessed the proportions of GPR15⁺ cells in the lamina propria. CD patients had a higher proportion of GPR15⁺ Tregs than Teffs (20.37±17.03 vs 12.83±10.77 p=0.0039). CD patients had similar proportions of GPR15⁺ Tregs in the lamina propria as HCs; however, there was a significant difference in the proportion of GPR15+Teffs (9.61 [4.56-18.8] vs 4.21 [3.02-8.27], p<0.05).

To complete our understanding of the dynamics of gut homing Treg and Teff in CD we assessed whether the proportions of α4β7⁺ Tregs and Teffs were affected by thiopurine or anti-TNF therapy. Neither thiopurine nor anti-TNF therapy appeared to affect the proportions of Treg or Teff licensed to traffic to the gut (FIG. 7), implying that trafficking and pro-inflammatory pathways are mechanistically separable.

RAR568 Induces a407 Mmore Efficiently and Robustly than ATRA

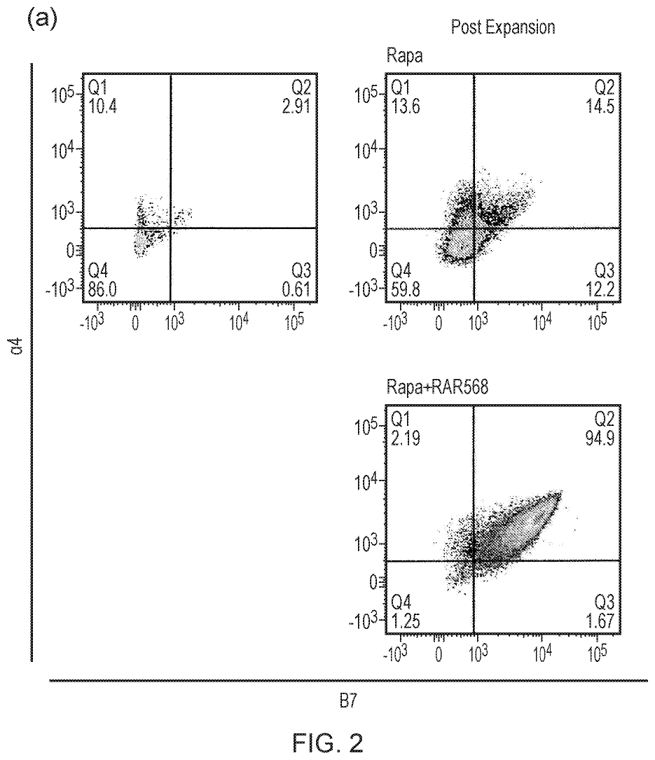

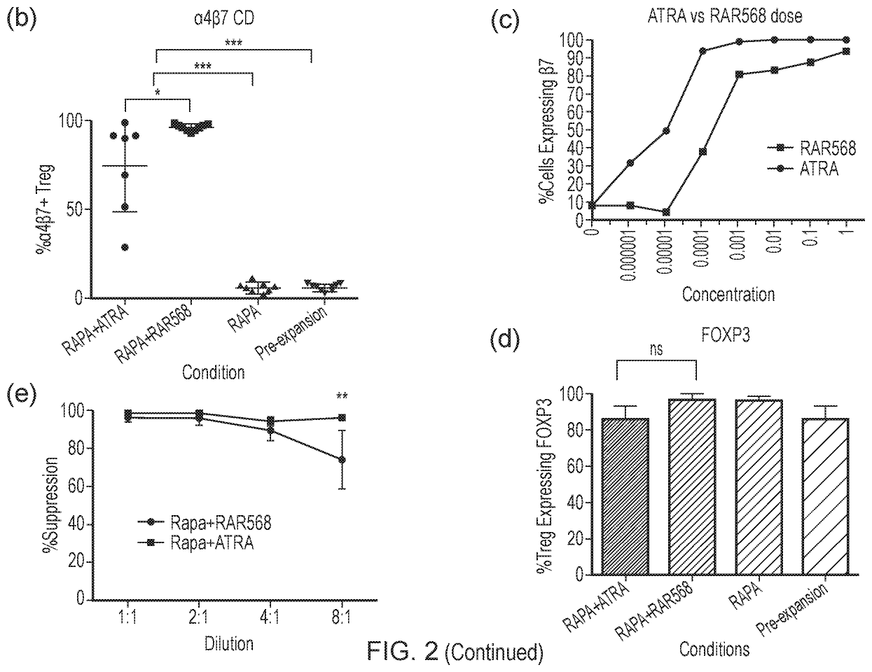

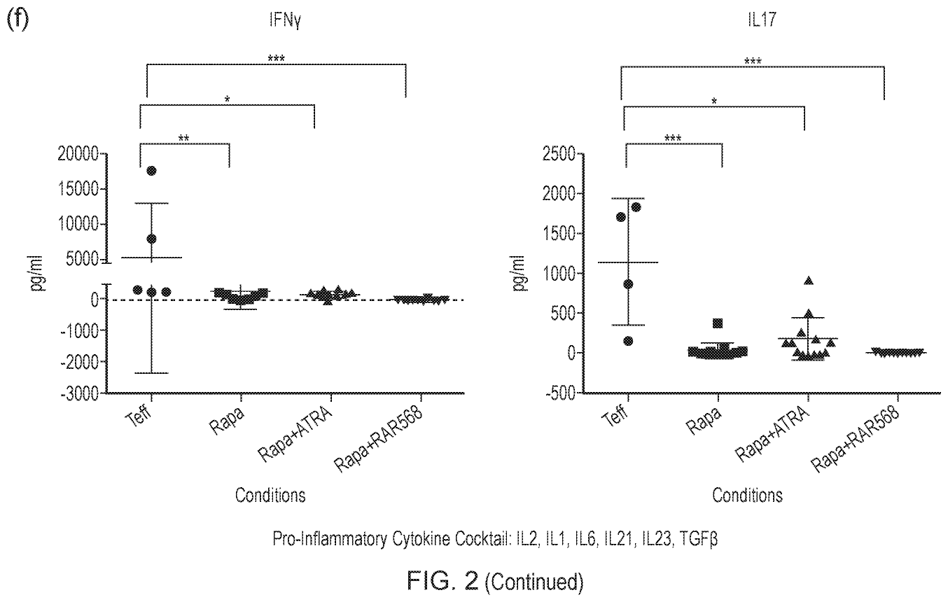

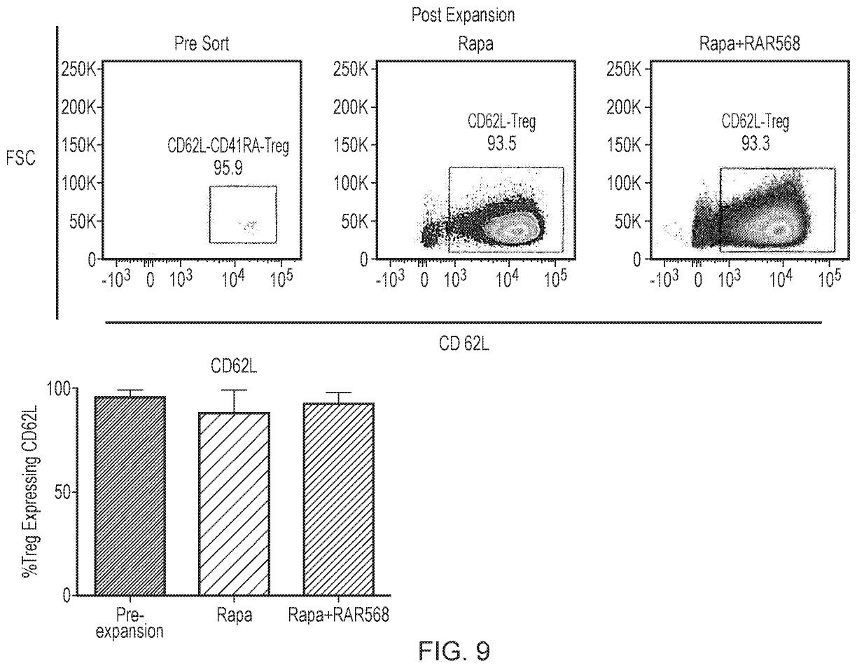

To address the balance between regulatory and effector T cells in the lamina propria of patients with CD, we sought to develop a highly suppressive, phenotypically stable population of autologous ex vivo expanded Tregs that were licensed to traffic to the gut by high level expression of α4β7. These cells could then be utilized as an autologous cell-based therapy for CD. We compared the efficacy of ATRA with RAR568 at inducing α4β7, to determine which agent would be more suitable for downstream application in a clinical trial of Treg therapy for CD. As previously defined²², our standard culture conditions used the CD4⁺CD25^{hi}CD127^{lo}CD45RA⁺ nïave Treg subset, cultured in the presence of rapamycin (RAPA) and high dose IL-2. When compared to cells cultured under standard conditions (Rapa), cells cultured under standard culture conditions but with the addition of RAR568 (Rapa+RAR568) expressed significantly more α4β7 (95.9±1.93 vs 5.947±3.18, p<0.0001; gating strategy is shown in FIG. 2a). Additionally, cells cultured in the presence of RAR568 cumulatively expressed more α4β7 than those cultured in the presence of ATRA (95.89±1.93 vs 74.21±25.89, p=0.024; FIG. 2b). The efficacy of RAR568 to induce the expression of integrin β7 was apparent at much lower concentrations, when compared to ATRA (FIG. 2c), with an EC50 of 0.01 nM/L for RAR568, versus an EC50 for ATRA of 1.5 nM/L. Importantly, the standard deviation of α4β7 expression for cells cultured in the presence of RAR568 was much lower than those cultured in the presence of ATRA (1.93 vs. 25.89), which has important implications for downstream quality control when these agents are employed for cell-based therapy. The expression of CD62L, required for homing to the lymph nodes and the effective interaction between integrin α4β7 and its ligand MAdCAM-1 was maintained following ex vivo expansion, irrespective of retinoid treatment (FIG. 9).

Treatment with RAR568 Does Not Affect Treg Stability or Suppressive Ability

Cells expanded in the presence of RAR568 express high levels of FOXP3 (96.99% ±3.51). This value is not significantly different to cells expanded under standard conditions (96.03±6.18) and those expanded in the presence of ATRA (86.15±19.88) (FIG. 2d). However, Tregs expanded in the presence of ATRA showed a less consistent level of FOXP3 expression (range 40.2-99.7, SD 19.88), when compared to those grown in the presence of RAR568 (range 91.5-99.8, SD 3.51).

Cells expanded in the presence of RAR568 were highly suppressive even at the lowest (8:1) titration. Conversely, cells grown in the presence of ATRA became less suppressive at the lowest titration (p<0.005) (FIG. 2e). Tregs expanded ex vivo in the presence of either ATRA or RAR568 did not produce IL-17 or IFNγ following pro-inflammatory cytokine challenge (FIG. 2f).

Treatment with RAR568 Avoids Off Target RARγ Effects and Skewing to a Pro-Inflammatory Phenotype.

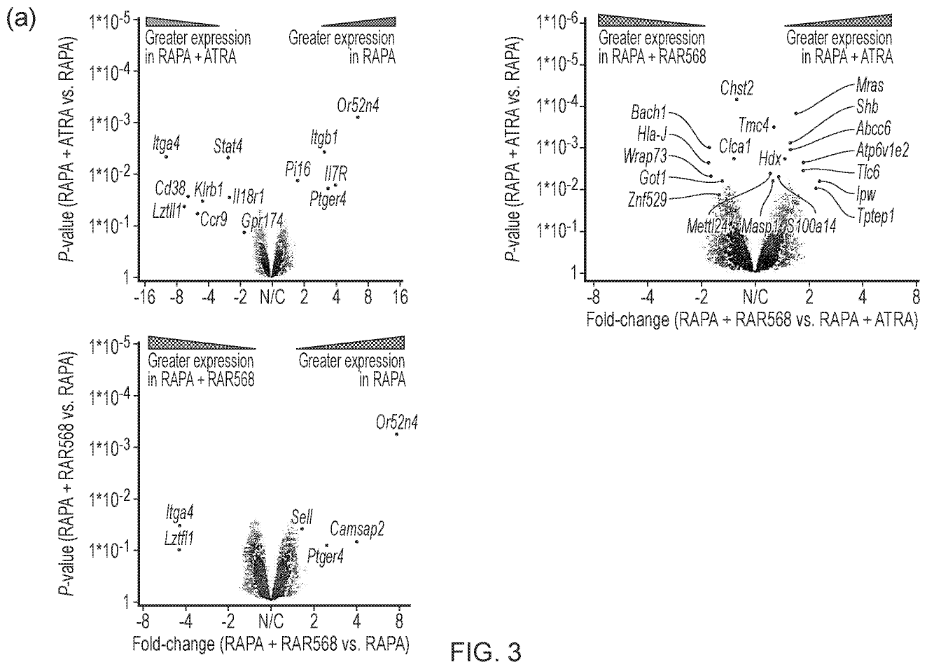

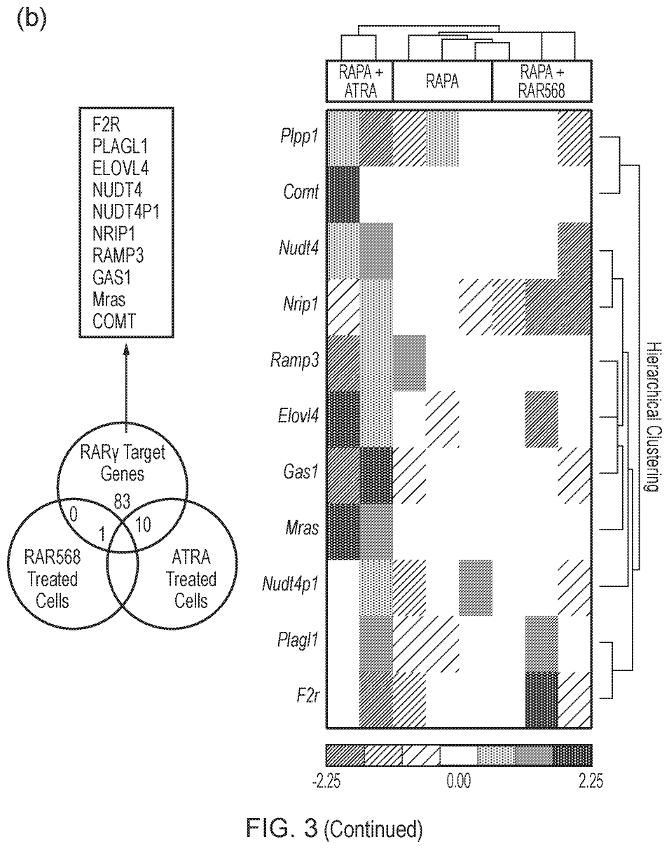

Gene expression analyses were performed on Tregs from CD patients expanded in the presence of Rapa+ATRA, Rapa+RAR568 or rapamycin only (n=3 in each group). A key difference between the ATRA-treated cells and RAR568-treated Tregs was a significant increase in transcripts for CD161 in the ATRA treated group compared to rapamycin only (p<0.05). CD161 has previously been described as a marker of T helper (Th) 17-like Tregs³². This was not observed in the RAR568-treated group. Additionally, Tregs treated with ATRA had a >2 fold increase in the expression of STAT4, IL18R1, CD38 and GPR174 (p<0.05) (FIG. 3a). IL-18 Receptor 1 and STAT4 are responsible for Th1 lineage commitment and IFNγ production, both have been independently identified as IBD disease related polymorphisms on GWAS³³⁻³⁶. CD38 has been identified as a marker associated with mature T cells, signaling reduced proliferation, but an increased ability to produce pro-inflammatory cytokines such as IFNγ³⁷. Ligation of GPR174 negatively affects Treg accumulation and function³⁸. No clear difference in transcripts for canonical pathways were identified when ATRA treated cells were compared with RAR568 treated cells.

To assess for off target RARγ effects, we compared the gene expression profiles of RAR568 and ATRA treated cells to a published dataset of RARγ target genes³⁹. Eleven out of 94 RARγ target genes were upregulated in the ATRA-treated samples, compared to only one in the RAR568-treated samples (FIG. 3b). Given the efficacy at inducing α4β7 and lack of off target effects, RAR568 fulfilled the target product profile for an agent that could be used for ex vivo Treg expansion for cell-based therapy purposes. Therefore, we probed this effect of RAR568 on Tregs from CD patients in functional in vitro and in vivo trafficking assays.

The Induction of α4β7 is functionally relevant in vitro and in vivo

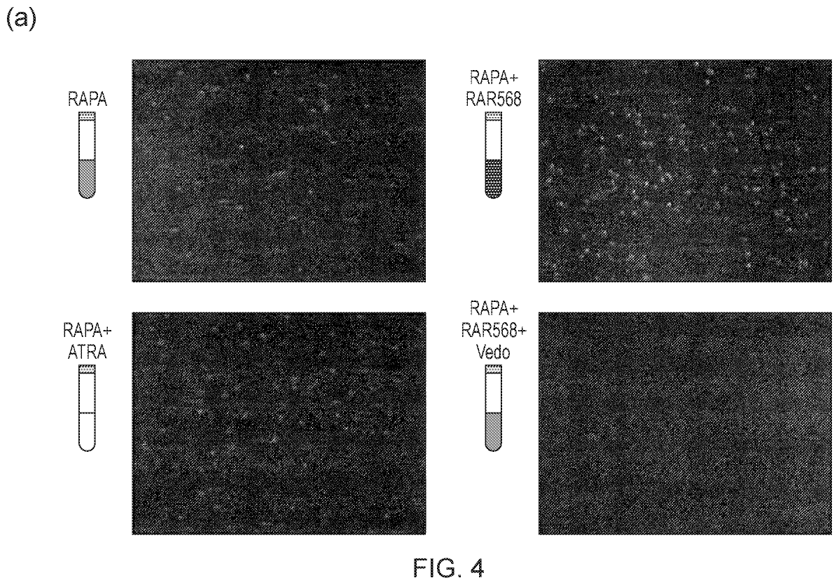

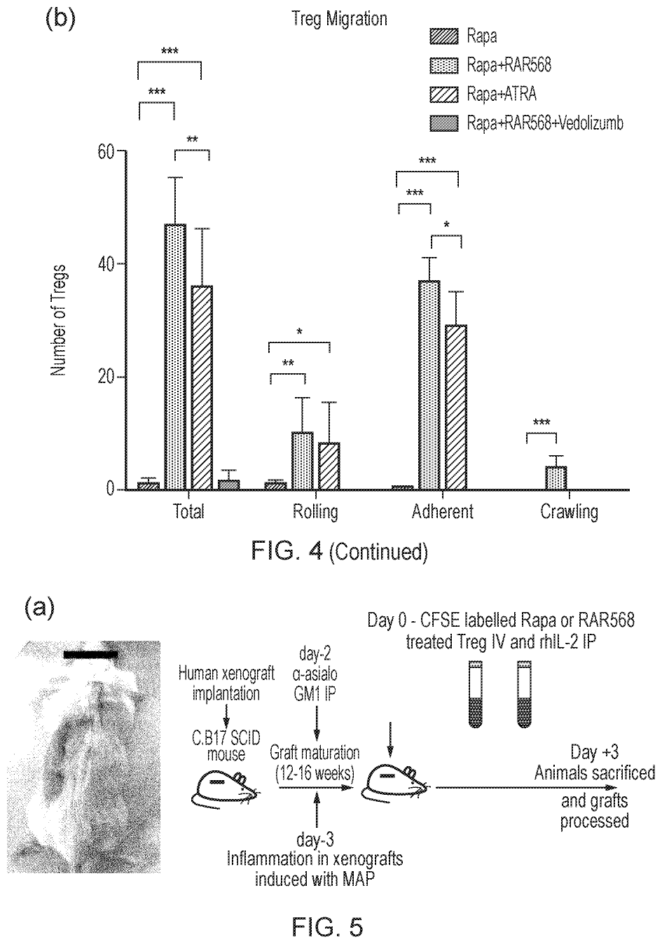

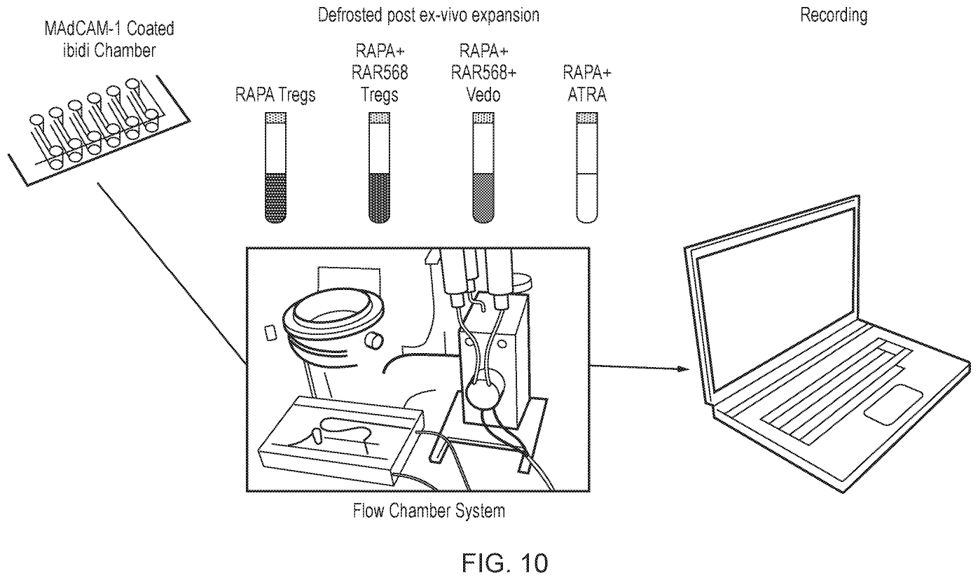

In order to assess the physiological relevance of the induction of α4β7 expression by RAR568, treated and untreated Tregs from CD patients were passed through a MAdCAM-1 coated flow chamber (FIG. 10). The total number of cells adherent to the chamber, as well as their stages of rolling, adhesion and crawling, were compared to cells that were expanded under standard conditions. There were significantly more total cells as well as cells at each condition of migration when RAR568-treated cells were passed through the chamber compared to their counterparts expanded under standard conditions. All stages of cell migration were blocked when the cells were treated with a monoclonal antibody to integrin α4β7 (Vedolizumab; FIG. 4). This demonstrates that not only is the induction of α4β7 relevant in vitro, but that it is dependent on the interaction of α4β7 and MAdCAM-1 under conditions of physiological shear flow, with maximum interaction induced by the selective ligation of RARα.

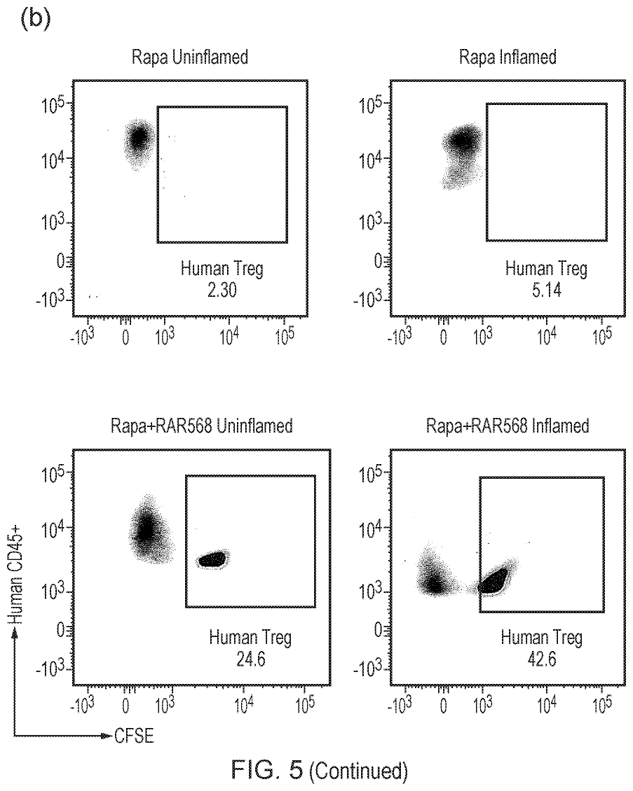

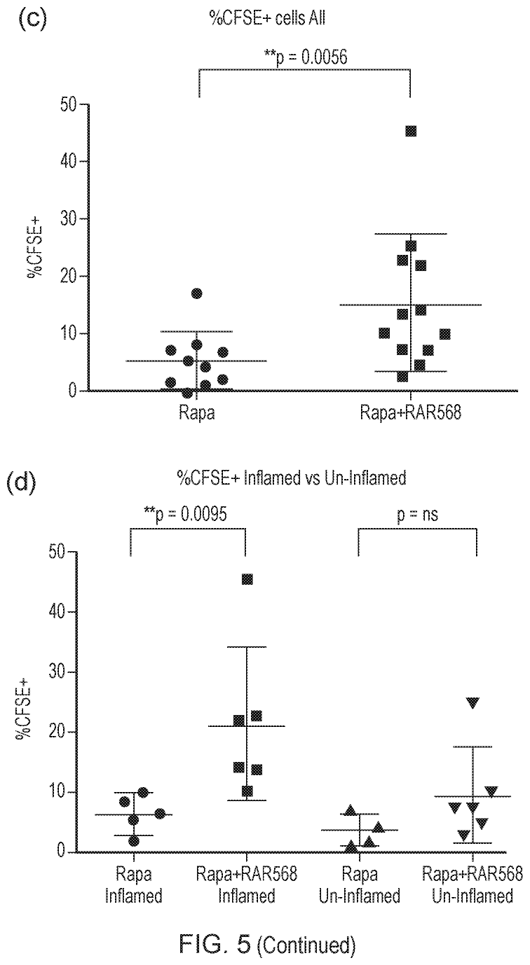

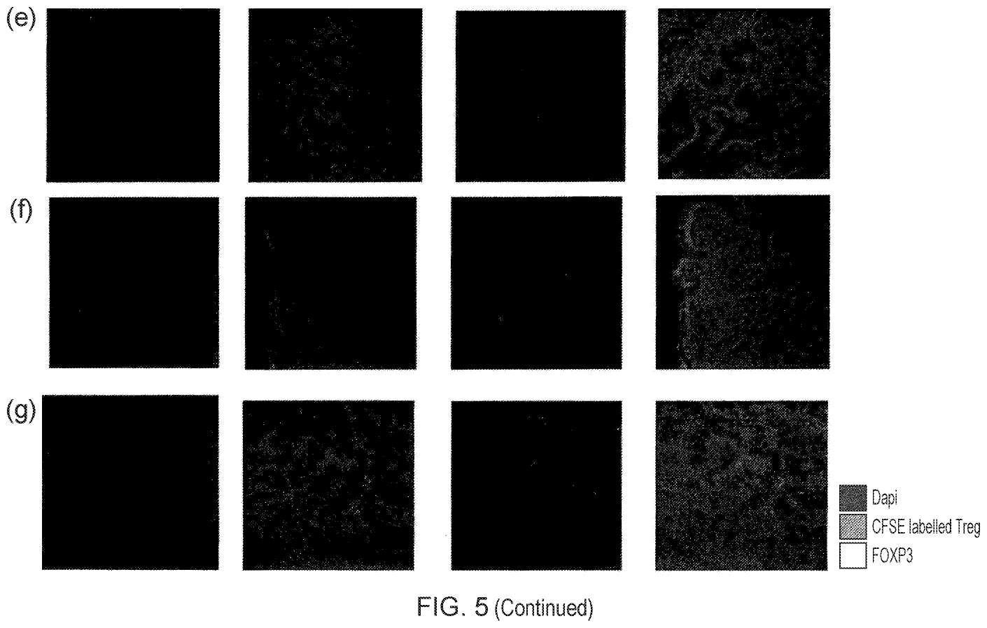

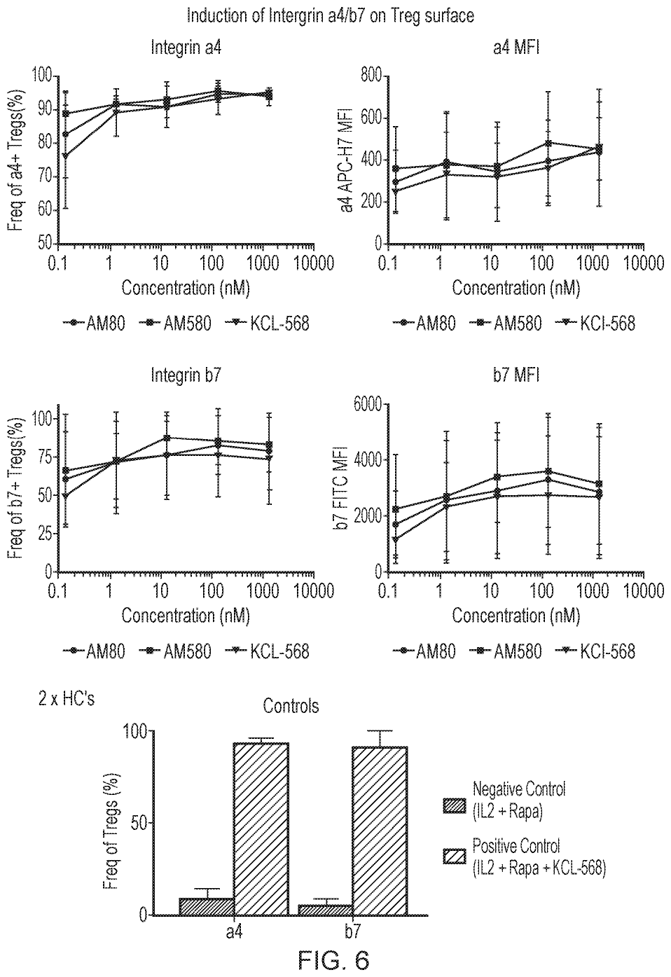

To assess if the induction of α4β7 was functionally relevant in vivo, cells treated with RAR568 or cells expanded under standard conditions were fluorescently labelled and transferred into a SCID mouse xenografted with human fetal intestinal small bowel by intra-venous injection. Inflammation was induced in the xenografts with Mycobactrium Avium Paratuberculosis (MAP). It has previously been demonstrated that MAP can infiltrate into the xenografts and induce inflammation detectable histologically and by the production of pro-inflammatory cytokines²⁹. Experimental design is illustrated in FIG. 5a.

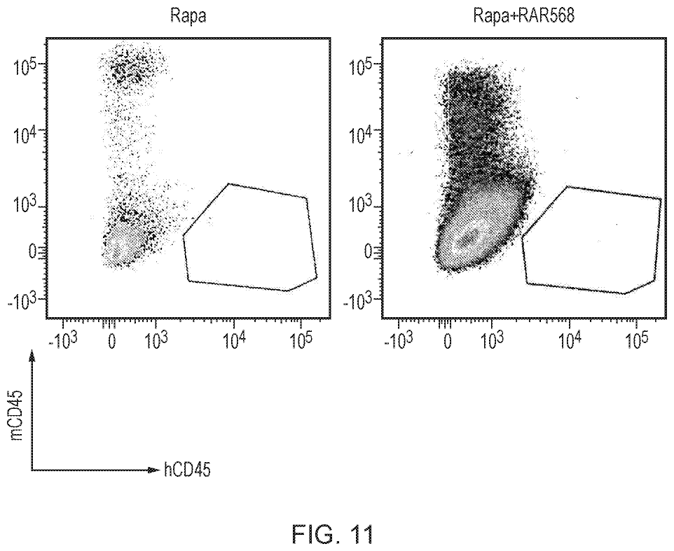

RAR568-treated cells were significantly more likely to traffic to xenografts 72 hours following Treg transfer compared to Tregs expanded under standard conditions (p=0.00560; representative FACS plots FIG. 5b, cumulative data FIG. 5c). The difference in Treg trafficking to the xenografts was further increased by the presence of inflammation; significantly more RAR568-treated cells trafficked to the inflamed xenografts than those grown under standard conditions (p=0.0095; FIG. 5d). The presence of CFSE labelled FOXP3⁺ Tregs was also evident in immunofluorescent labelled cryosections from the inflamed xenografts of mice which had received the RAR568-treated cells (FIG. 5g), but not in the xenografts of controls or those who received Rapa-treated cells (FIG. 5e-f). Given the concerns that adoptively transferred human cells may be located outside the gastrointestinal system, we assessed Treg trafficking to the spleen. There were no human CD45 positive cells found in the spleens of mice treated with cells either grown under standard conditions or those treated with RAR568 (FIG. 11).

Discussion

Contrary to previous reports¹⁸, we found integrin β7 to be more highly expressed on effector T cells in the peripheral blood of CD patients rather than on regulatory T cells. Furthermore, patients with active CD have a significantly lower proportion of circulating α4β7⁺ Tregs than their HC counterparts, and a significantly higher proportion of Teff licensed to traffic to the gut. This deficiency does not affect all gut homing receptors, with CD patients having a comparable proportion of GPR15⁺ Tregs in the circulation and a higher proportion of CCR9⁺ Tregs than their HC counterparts. The reduction in the proportion of α4β7⁺ Tregs is also not a function of a global reduction in the proportion of circulating Tregs, as there is a higher proportion of circulating Tregs in patients with active CD compared to HCs. Thus, while the absolute difference in α4β7⁺ Treg proportions between CD patients and HCs controls is small, the fact that this difference does not exist with any other marker in addition to the fact that the α4β7 pathway is already being therapeutically exploited with monoclonal antibodies for the treatment of CD, would suggest that this difference is significant. The Treg/Teff imbalance is also apparent in the lamina propria of CD patients. There is a higher proportion of α4β7⁺ Teffs in CD, whereas in HCs there is an equal balance between α4β7⁺ Tregs and Teffs. A limitation of this finding was that the HC LPMC donors were older than their CD counterparts, this is an unavoidable function of the patients who present for a colonoscopy in the absence of CD. Studies of Tregs in older subjects have suggested an increase in natural Treg and a decline in iTreg⁴⁰. Whilst this may explain a better balance between Tregs and Teffs in HCs, it does not explain the lower Treg and significantly lower Teff numbers seen in HC compared to CD patients. The imbalance between gut homing Tregs and Teffs could be a potential pathogenic mechanism underlying the disease. Thus, it would follow that by therapeutic expansion of the circulating population of Tregs that is licensed to home to the inflamed bowel, we could re-set the balance between regulatory and effector T cells in this organ which might contribute to disease resolution.

The profound and consistent induction of α4β7 by RAR568 confers Tregs with the ability to traffic to the diseased organ for which they are therapeutically destined. A far more robust induction of α4β7 by RAR568, a highly specific agonist of RARα, is consistent with the fact that it is the downstream function of this receptor, rather than RARβ or RARγ⁴¹. Although standard retinoic acid (ATRA) is somewhat effective at inducing the expression of integrin α4β7, there are ongoing concerns about the ability of ATRA to also skew Tregs towards a pro-inflammatory phenotype^{13, 25}. ATRA can interact with RARα, RARβ and RARγ, however it has a much higher affinity for RAR γ. The higher standard deviation observed in FOXP3 expression, IL17 and IFNγ production when cells are treated with ATRA compared to RAR568 suggests that ATRA's previously noted ability to skew cells towards a pro-inflammatory phenotype may be due to activation of RARγ and could therefore be avoided when using a RARα specific agonist. It could be argued that the observed heterogeneity in FOXP3 expression is simply due to sample purity, however, given that the expansion in the presence of ATRA or RAR568 took place side by side from a sample that was derived from the same donor and therefore underwent an identical flow sorting protocol, this possibility is less likely.

The increased expression of CD161 transcripts in ATRA-treated cells demonstrates that they may be skewed towards a Th17-producing phenotype. As an immune imbalance skewed towards a Th17 response has been implicated in the pathogenesis of CD⁴², it would be imprudent to introduce an expanded cell population that has the ability to secrete IL17 into the inflamed gut of CD patients. Similarly, the induction of STAT4 and IL-18R1 and CD38 on ATRA treated cells, may confer them with an increased ability to skew to a Th1 like phenotype under pro-inflammatory conditions and secrete IFNγ. Given the aim of treating cells with ATRA in vitro is to induce migration to the gut, the induction of GPR174 by ATRA, which impedes Treg migration, would hinder that aim. The near complete lack of induction of RARγ target genes in the RAR568-treated cells further confirms the alpha selectivity of the agonist thus allowing us to feel confident that we will not see any off target effects when it is used for large scale Treg manufacture in clinical trials.

Patient-derived Tregs grown under standard conditions do express low levels of α4β7, however, they displayed negligible levels of rolling, adherence and activation when presented with MAdCAM-1 in the Ibidi flow chamber experiments. By contrast, RAR568-treated cells interacted very efficiently with this ligand and to a much greater extent than cells treated with the nonselective RAR agonist ATRA. This suggests that high levels of α4β7 expression are required in order for a cell to progress through the stages of endothelial migration. The complete blockade of interaction between MAdCAM-1 and RAR568-treated Tregs in the flow chamber by treatment with Vedolizumab proves that this process is dependent on α4β7. Taken together, we propose that when RAR568-treated cells are transferred into a pro-inflammatory environment, they will home to tissues where MAdCAM-1 is upregulated, such as the inflamed gut in CD.

To confirm further that ex vivo expanded Tregs remain viable in vivo and have the ability to migrate to the inflamed bowel, we transferred cells grown either under standard conditions or in the presence of RAR568 into a SCID mouse xenografted with human foetal small bowel. The grafts in this model are known to express MAdCAM-143 and develop into tissue that is functionally and morphologically identical to normal adult human gut^{28, 29}. MAP was chosen to induce inflammation in the xenografts as it causes granulomatous inflammation, which provides a suitable model for the inflammation occurring in CD. Furthermore, the ability of MAP to invade goblet cells and induce inflammation in this model has been previously described²⁹.

Significantly more RAR568-treated cells found their way into the xenografts, particularly when inflammation was induced. We can therefore surmise that by inducing the expression of MAdCAM-1 the inflammatory process in this model draws more of the RAR568-treated cells, which are uniformly α4β7⁺, to the inflamed xenografted human gut. This parallels our in vitro findings and would suggest that after treatment with RAR568, Tregs will home to the inflamed gut when they are administered in upcoming trials of cell-based therapy for CD.

### REFERENCES

1. Sakaguchi S, Ono M, Setoguchi R, et al. Foxp3+CD25+CD4+ natural regulatory T cells in dominant self-tolerance and autoimmune disease. Immunol Rev 2006;212:8-27.

2. Himmel M E, Hardenberg G, Piccirillo C A, et al. The role of T-regulatory cells and Toll-like receptors in the pathogenesis of human inflammatory bowel disease. Immunology 2008;125:145-53.

3. Izcue A, Coombes J L, Powrie F. Regulatory lymphocytes and intestinal inflammation. Annu Rev Immunol 2009;27:313-38.

4. Huang H, Fang M, Jostins L, et al. Fine-mapping inflammatory bowel disease loci to single-variant resolution. Nature 2017;547:173-178.

5. Di Ianni M, Falzetti F, Carotti A, et al. Tregs prevent GVHD and promote immune reconstitution in HLA-haploidentical transplantation. Blood 2011;117:3921-8.

6. Valencia X, Yarboro C, Illei G, et al. Deficient CD4CD25high T regulatory cell function in patients with active systemic lupus erythematosus. J Immunol 2007;178:2579-88.

7. Trzonkowski P, Bieniaszewska M, Juscinska J, et al. First-in-man clinical results of the treatment of patients with graft versus host disease with human ex vivo expanded CD4+CD25+CD127− T regulatory cells. Clin Immunol 2009;133:22-6.

8. Sakaguchi S, Miyara M, Costantino C M, et al. FOXP3+ regulatory T cells in the human immune system. Nat Rev Immunol 2010;10:490-500.

9. Miyara M, Yoshioka Y, Kitoh A, et al. Functional delineation and differentiation dynamics of human CD4+ T cells expressing the FoxP3 transcription factor. Immunity 2009;30:899-911.

10. Sagoo P, Lombardi G, Lechler R I. Regulatory T cells as therapeutic cells. Curr Opin Organ Transplant 2008;13:645-53.

11. Katoh H, Zheng P, Liu Y. FOXP3: genetic and epigenetic implications for autoimmunity. J Autoimmun 2013;41:72-8.

12. Canavan J B, Afzali B, Scotta C, et al. A rapid diagnostic test for human regulatory T-cell function to enable regulatory T-cell therapy. Blood 2012;119:e57-66.

13. Scotta C, Esposito M, Fazekasova H, et al. Differential effects of rapamycin and retinoic acid on expansion, stability and suppressive qualities of human CD4(+)CD25(+)FOXP3(+) T regulatory cell subpopulations. Haematologica 2013;98:1291-9.

14. Afzali B, Edozie F C, Fazekasova H, et al. Comparison of regulatory T cells in hemodialysis patients and healthy controls: implications for cell therapy in transplantation. Clin J Am Soc Nephrol 2013;8:1396-405.

15. Brunstein C G, Miller J S, Cao Q, et al. Infusion of ex vivo expanded T regulatory cells in adults transplanted with umbilical cord blood: safety profile and detection kinetics. Blood 2011;117:1061-70.

16. Marek-Trzonkowska N, Mysliwiec M, Dobyszuk A, et al. Administration of CD4+CD25highCD127− regulatory T cells preserves beta-cell function in type 1 diabetes in children. Diabetes Care 2012;35:1817-20.

17. Sandborn W J, Feagan B G, Rutgeerts P, et al. Vedolizumab as Induction and Maintenance Therapy for Crohn's Disease. New England Journal of Medicine 2013;369:711-721.

18. Fischer A, Zundler S, Atreya R, et al. Differential effects of alpha4beta7 and GPR15 on homing of effector and regulatory T cells from patients with UC to the inflamed gut in vivo. Gut 2016;65:1642-64.

19. Saruta M, Yu Q T, Fleshner P R, et al. Characterization of FOXP3+CD4+ regulatory T cells in Crohn's disease. Clin Immunol 2007;125:281-90.

20. Mayne C G, Williams C B. Induced and natural regulatory T cells in the development of inflammatory bowel disease. Inflamm Bowel Dis 2013;19:1772-88.

21. Hovhannisyan Z, Treatman J, Littman D R, et al. Characterization of interleukin-17-producing regulatory T cells in inflamed intestinal mucosa from patients with inflammatory bowel diseases. Gastroenterology 2011;140:957-65.

22. Canavan J B, Scotta C, Vossenkamper A, et al. Developing in vitro expanded CD45RA+ regulatory T cells as an adoptive cell therapy for Crohn's disease. Gut 2016;65:584-94.

23. Brown C C, Esterhazy D, Sarde A, et al. Retinoic acid is essential for Th1 cell lineage stability and prevents transition to a Th17 cell program. Immunity 2015;42:499-511.

24. Lu L, Lan Q, Li Z, et al. Critical role of all-trans retinoic acid in stabilizing human natural regulatory T cells under inflammatory conditions. Proc Natl Acad Sci US A 2014;111:E3432-40.

25. Golovina T N, Mikheeva T, Brusko T M, et al. Retinoic acid and rapamycin differentially affect and synergistically promote the ex vivo expansion of natural human T regulatory cells. PLoS One 2011;6:e15868.

26. Schneider S M, Offterdinger M, Huber H, et al. Activation of retinoic acid receptor alpha is sufficient for full induction of retinoid responses in SK-BR-3 and T47D human breast cancer cells. Cancer Res 2000;60:5479-β7.

27. Clarke E, Jarvis C I, Goncalves M B, et al. Design and synthesis of a potent, highly selective, orally bioavailable, retinoic acid receptor alpha agonist. Bioorg Med Chem 2017.

28. Howie D, Spencer J, DeLord D, et al. Extrathymic T cell differentiation in the human intestine early in life. J Immunol 1998;161:5862-72.

29. Golan L, Livneh-Kol A, Gonen E, et al. Mycobacterium avium paratuberculosis invades human small-intestinal goblet cells and elicits inflammation. J Infect Dis 2009;199:350-4.

30. Maul J, Loddenkemper C, Mundt P, et al. Peripheral and intestinal regulatory CD4+ CD25(high) T cells in inflammatory bowel disease. Gastroenterology 2005;128:1868-78.

31. Reikvam D H, Perminow G, Lyckander L G, et al. Increase of regulatory T cells in ileal mucosa of untreated pediatric Crohn's disease patients. Scand J Gastroenterol 2011;46:550-60.

32. Afzali B, Mitchell P J, Edozie F C, et al. CD161 expression characterizes a subpopulation of human regulatory T cells that produces IL-17 in a STAT3-dependent manner. Eur J Immunol 2013;43:2043-54.

33. O'Malley J T, Eri R D, Stritesky G L, et al. STAT4 isoforms differentially regulate Th1 cytokine production and the severity of inflammatory bowel disease. J Immunol 2008;181:5062-70.

34. Xu J, Yang Y, Qiu G, et al. Stat4 is critical for the balance between Th17 cells and regulatory T cells in colitis. J Immunol 2011;186:6597-606.

35. Barrett J C, Hansoul S, Nicolae D L, et al. Genome-wide association defines more than 30 distinct susceptibility loci for Crohn's disease. Nat Genet 2008;40:955-62.

36. Hedl M, Zheng S, Abraham C. The IL18RAP region disease polymorphism decreases IL-18RAP/IL-18R1/IL-1R1 expression and signaling through innate receptor-initiated pathways. J Immunol 2014;192:5924-32.

37. Sandoval-Montes C, Santos-Argumedo L. CD38 is expressed selectively during the activation of a subset of mature T cells with reduced proliferation but improved potential to produce cytokines. J Leukoc Biol 2005;77:513-21.

38. Barnes M J, Li C-M, Xu Y, et al. The lysophosphatidylserine receptor GPR174 constrains regulatory T cell development and function. The Journal of Experimental Medicine 2015;212:1011-1020.

39. Su D, Gudas L J. Gene expression profiling elucidates a specific role for RARgamma in the retinoic acid-induced differentiation of F9 teratocarcinoma stem cells. Biochem Pharmacol 2008;75:1129-60.

40. Jagger A T, Shimojima Y, Goronzy J J, et al. T regulatory cells and the immune aging process. Gerontology 2014;60:130-7.

41. Kang S G, Park J, Cho J Y, et al. Complementary roles of retinoic acid and TGF-betal in coordinated expression of mucosal integrins by T cells. Mucosal Immunol 2011;4:66-82.

42. Abraham C, Cho J. Interleukin-23/Th17 pathways and inflammatory bowel disease. Inflamm Bowel Dis 2009;15:1090-100.

43. Winter H S, Hendren R B, Fox C H, et al. Human intestine matures as nude mouse xenograft. Gastroenterology 1991;100:89-98.

1. Method for making regulatory T cells (Tregs) with improved functionality, comprising contacting Tregs derived from a subject with an immune-mediated gut disorder with at least one RARα agonist, functional analogue or derivative thereof, wherein the RARα agonist is selective for RARα over RARβ or RARγ.

2. Method according to claim 1, wherein said Tregs are obtained from a biological sample, such as peripheral blood, thymus, lymph nodes, spleen, bone marrow.

3. Method according to claim 2, wherein said Tregs are isolated from said biological sample, optionally by cell sorting, suitably flow cytometry.

4. Method according to claim 3, wherein said isolated Tregs are expanded and during said expansion are contacted with an effective amount of at least one RARα agonist, functional analogue or derivative thereof.

5. Method according to claim 4 which is followed by the step of obtaining ex vivo expanded Tregs with improved functionality.

6. Method according to claim 5, wherein said ex vivo expanded Tregs are introduced into a subject suffering from an immune-mediated gut disorder, which may be the same subject from which the biological sample containing the Tregs was obtained.

7. Method according to claim 6 which is followed by monitoring for or detecting a resulting improvement in the disorder in the subject.

8. Method according to any preceding claim, wherein said improved Treg function comprises increased capacity for gut-homing, and/or increased expression of α4β7 integrin and/or increased expression of CCR9 and/or improved Treg retention and/or increased potency.

9. Method according to any preceding claim, wherein said RARα agonist has a greater specificity for RARα than RARβ or RARγ.

10. Method according to claim 9, wherein said RARα agonist is selected from the group consisting of: RAR568, AM580, AM80 (tamibarotene), RX-195183, BMS753, BD4, AC-93253, and AR7.

11. Method according to any preceding claim, wherein said immune-mediated gut disorder is selected from: inflammatory bowel disease (IBD), particularly Chron's Disease (CD); or colitis, such as ulcerative colitis (UC), checkpoint-related colitis, treatment-resistant Clostridium difficile-associated colitis, or GvHD, where the gut is involved, celiac disease; autoimmune gastritis.

12. Method according to any preceding claim, wherein the Treg is selected from: a CD4+CD25+FOXP3+ T cell; a CD4+CD25+CD127−/low T cell; a CD4+CD25+FOXP3+CD127−/low T cell; a CD4+CD25+CD127−CD45RA+ T cell; a CD4+CD25+CD127lowCD45RA+ T cell; a CD4+CD25+CD127lowCD45RA-CD45RO+ T cell; a CD4+CD25+CD127lowCD45RA+CD45RO+ T cell.

13. Ex vivo expanded Tregs obtainable by a method according to any one of claims 1 to 11 and having increased capacity for gut-homing, and/or increased expression of α4β7 integrin and/or improved Treg retention and/or increased potency.

14. Modified Tregs modified to (over)express a gut-homing molecule, particularly α4β7 integrin and/or CCR9.

15. Modified Tregs according to claim 14 having increased capacity for gut-homing, and/or increased expression of α4β7 integrin and/or increased expression of CCR9 and/or improved Treg retention and/or increased potency.

16. Modified Tregs according to claim 14 or 15, wherein said Tregs are modified by gene editing or by introducing into an unmodified Treg (e.g. by transduction or transfection) a polynucleotide or vector comprising at least one gut-homing molecule, optionally wherein the gut-homing molecule is selected from α4β7 integrin and/or CCR9.

17. Pharmaceutical composition comprising Tregs according to any one of claims 13 to 16 for the treatment, amelioration or prevention of an immune-mediated gut disorder.

18. Method of treating an immune-mediated gut disorder, comprising administering ex vivo expanded Tregs according to claim 13 and/or modified Tregs according to any one of claims 13 to 16 and/or a pharmaceutical composition according to claim 17 to a subject having an immune-mediated gut disorder.

19. Method of treatment according to claim 18, wherein said treatment restores to more equal levels of α4β7+ Treg and Teff compared to levels prior to treatment.

20. Ex vivo expanded Tregs according to claim 13, modified Tregs according to any one of claims 13 to 16, a pharmaceutical composition according to claim 17, RARα agonists and analogues and derivates thereof for use in the treatment, amelioration or prevention of an immune-mediated gut disorder.

21. Use according to claim 20, wherein said immune-mediated gut disorder is selected from: inflammatory bowel disease (IBD), such as Chron's Disease (CD); or colitis, such as ulcerative colitis (UC), checkpoint-related colitis, treatment-resistant Clostridium difficile-associated colitis, or GvHD, where the gut is involved, celiac disease; autoimmune gastritis.

22. Culture and/or expansion media for use in the production of ex vivo expanded Tregs, which media comprise at least one RARα agonist, functional analogue or derivative thereof.


SEQUENCE LISTING (8 sequences)

SEQ ID NO: 1 - DNA, 82037 bp, Homo Sapiens
ataacgtctttgtcactaaaatgttccccaggggccttcggcgagtctttttgtttggttttttgtttttaatctgtggctcttgataatttatctagtggttgcctacacctgaaaaacaagacacagtgtttaactatcaacgaaagaactggacggctccccgccgcagtcccactccccgagtttgtggctggcatttgggccacgccgggctgggcggtcacagcgaggggcgcgcagtttggggtcacacagctccgcttctaggccccaaccaccgttaaaaggggaagcccgtgccccatcaggtccgctcttgctgagcccagagccatcccgcgctctgcgggctgggaggcccgggccaggacgcgagtcctgcgcagccgaggttccccagcgccccctgcagccgcgcgtaggcagagacggagcccggccctgcgcctccgcaccacgcccgggaccccacccagcggcccgtacccggagaagcagcgcgagcacccgaagctcccggctggcggcagaaaccgggagtggggccgggcgagtgcgcggcatcccaggccggcccgaacgctccgcccgcggtgggccgacttcccctcctcttccctctctccttcctttagcccgctggcgccggacacgctgcgcctcatctcttggggcgttcttccccgttggccaaccgtcgcatcccgtgcaactttggggtagtggccgtttagtgttgaatgttccccaccgagagcgcatggcttgggaagcgaggcgcgaacccggcccccgaagggccgccgtccgggagacggtgatgctgttgctgtgcctgggggtcccgaccggccgcccctacaacgtggacactgagagcgcgctgctttaccagggcccccacaacacgctgttcggctactcggtcgtgctgcacagccacggggcgaaccgatggtgagtagagttggactgatgcgccctcagcagctcagagcggcgtgagaatggcgccctagggattccctgcccgattcaaactttcctccctcccaaggaggcaggagggaaacgctgcgagagccagctcgctggaaatctgccagggaaactaacttatcttggggcggcagcgccccgggtgcgttatggtgcaaggtcttggagcttcgagtcgggggtggtgggcggaggaggaggtggggaagctgccctgctgcggactgcacatctgtggcgacagggagctcagtgggcagcacagctcacgtctgagcgcacgtgcacgtgtctcgctttaggctcctagtgggtgcgcccactgccaactggctcgccaacgcttcagtgatcaatcccggggcgatttacagatgcaggatcggaaagaatcccggccagacgtgcgaacagctccagctgggtgagttgggtatgggaccaggagttagtgacctcccgaccccccatgtggaccccaccatagggcaagtcctggaaagattcacgttgcctgttacttctggtttaaagagcataaagttttcagtttcaactccatctcatcttgcctccttaatataaaagggattccctttctgttaatatcgttctcccttttccttatggcaatgattgcagtactctgacaactatgctagagaaatttcttatgataccttctcttcatctctccccgtctctccgtgtgtaaagcacattttagactctctcagcgtattaaggacaggatccagctaagccaagacttgagaattttatttccatctttgacctatcaaccctcttaaagaacaggctaaggagcatatcaggaatagcaattggaattgatcccatatcccgtcgtttaacctaaaggcctaaaaaaaaaagtgatagttttacttacaatatacaatctaaaagaaaaggaaaattggcatcttcatcgttaatgtagtataattttccattcccaaaaccgtcatccaaaactcatgttatatgggtccatctttttgcagtcatttctcaatgtctcagatttgtgacaagttgaaaaatgtgtactgatagaggggagaagtttcatgtttcaaagccaaagacaagtttgttgcacacaggcaagtggggaaagaggaaggaatattaaataacaagacctatcttgttccaaagtgttactgctatatatattgcttaacttttttccccttaaagcaaccctgggtcatggttgacattgttcctagtttatgtatgaggactgtggctcagagttgcaaatacctttttaaaagttacttagctagtgactgataggtgggaactaaagtcaaactagggtctgaatgcgtccaaagtctaagccattttcttccatgtatgtatcttcaatgcaaataaaattaagtgaatggtttaatatgttgtctgctgagctgatgggtcatctgtaaacggtgaataaaattggtacttctttatatttctttgctataaatggacttttgagattagttattggatctttaacttcttttgtattttatagtagtctttgctctttttcaaactgtgaagctggttcccttcattcagatgaattcagatagagtataaacataatggaatttttacttttaactcagagcacctataaaagtctacgagttgtctgtagcatgtagaataagccctagcctcttgttaagcatcctcaataccagaaatcacagtaggaatgcataaaacattcacagacccatacattttggaatgattttgcaaagcataattttcattgcatgagagagttgtgggtgaagaattagggagtggagccgacaactttggtctgccagaggatcttggcaaggtctcaggagggagcaattttatttctgttgtcctgtcatagtttcctgatccatgacattataactactgatatttagtgcctacttcctgtctggatggacactgaatctatttctggatgtctgtgtttaactggataaatcatgacataatactgtattaaatttaagataatgcctgtcaatggaaggaaaaaattgtttgggcatgtgtagcatgggagttgactgttctcagagaaatgtaaacatagtagatatgtcatagatatgaattaaatgaagatcttttggatggatgaatgaataaaagtggagtgaaaatttatcaacctgactgatgagtaagataattgattagaaatgatgagccaaactgatttttaactttcaaaattttggccaatctacaaggtatttactgggatgaaaggaaaaaaggcaagattcctagtcagtcaatgagttaaaacattaatagtgatagtaatgccttgagtatttgccgaagagttgactgaagttatttctcatgtaacattctaaagaatacagatatcttttattcctcatgtaaagaaactgcctctctattcagaataaattcagtggtagcttgaagctataaaatagggattgaggctacagtctagaaataaaacactaaaaacaatacattggtaataaagataatttaggattcttaggtttgtgggctatgcactattttgtatgaagaagtttcaagagtgtgtgtaaatatatataagccatcttctgtagaagagtgtgtgtgtgtgtgtgtgtgtgtgtgtgtgtgtgtgaatcttctgtataaaatagcttcagttgtcctgtttcaaatgtgggtgactctaatgtttcaaaaatgcttagctcagtactgacacataatacattcaaaaaatagtggttcttctttattgtttacttaaatctattatctgtatgttatctgtaacaagcaggctgaggttaatagggacaattgataaactgaaccaaaagtttaaaaattaaaaggtaatacattaaaaggtagaagttgaaatgaaacacaatgagaaatttttgttggctgaagaaaaaaatgttataatgattaaagatcgatctgtgattgccatgtcagtaaggtatcttctttcctttggaatagctttacttgctatcaagaacctgaaggtgtagggtcttcaaatggtgcccatctcccacacaattaaaaatatccacatggactagtctttgagtctctgtagaatttcagatcagtggttgattcagatagactcctcccctcaaaagagaaggtccatatctcgcctaccctaccctctgtggacaaggtgtcccatatggtatattcatgccataagcagaagtgagcctgcctgtttatctagaatgcccagggtgagagacgttaggcatgagataccccttttagtcaagcaagttgactgcagagccagcaccaagagagttgcctggatagtaccgcaaggtgcttttcttcctgcatatatattgttggcctccttttacctccctgactccccaagagacatctcttctctcacttcttttgtaccagaggaaagtggtctctctgccaggagggtctgggcccagacttgctcgtgaagactatatctgaggagacccaggtgatttctacatgtccactttccacccgggcactctgcatcatcaggcttgtttaaaggaaactttaatcacagctggttcctaaattaaggtgtcacaaataatgactcaattaatatagttaagcatagtccatccatggactatcaatattcaaatagaacataataaaacatagaaatgattatcaaattacactaaaaagatggtctttatttggttattcaaatatgtttccagatctttcattactttggagttaccactgaaagcattatacacacactaactctcatttgggtaatagtctaaagttatacaattacactatccagtatagtatccactaaccacaggtggctttgaacacataaaatgtggctagtcagcgatgagagggctgtaatgtaaattacatactggcttttgaatacttagtataagaaaactcattaatttttatgatatatattgaaacaaacactttaaaattattgattacataatattttggatttattaggcgaaataaaattttaggttaaataaaattaattttatttttacttttttcatgtggctattataaaatttaaaatttccaatgggcttgtattatatttctattggacagcactgctgtagagaaacattaagttctctgtgtaaaaataacgctatctctttaggagatgctttcctgatatttatgcataaatgagtagaactttcactttcttttctagactaagtgttctatagttactgggccttcaagctgcctgcctgtccctctcttacaggaaagacccatctcaagttagtcaccattacccatatttagatttctttctggaaattttattaccttagccagcctcctggttcatattatataacctatttgaaagaatttaattagcattgcattattaaactaatgattacctaatcctcacttctacttgattcataggagttattagcttaacattaaatagcaaatatctcactgttgaaacttcaaatatgtttttcttgataactttttactttttcatttgcaatgtgctttaaattgtagcttatgaagctgagattgcagattggtcattttgaaaaatcatccctgaatgaaggctgttatacaattatgtagaaaatcaagacagagagcttcactaataaactatcaggttaccagaaagaagtttccttatttccagcagtttcccaaactttatgtcccaaaaggatgaggtttccacctaatttgagagatgaggctttctaaatgcactttagtttttgccttaagtttacttggtcactgagtgatatacccaatgacttgaaaatatatccccttggcaacagttctagggtcagatgctgaaatccgttgcacatgtgggtaatttatccattcagttgttcattgagggatgcctaagggcctgaacctgggcatacaaaagaaacatactccttgtcccaaaggctttaaagtttataagataggttattataggaaagtatggtgagatttaatctaggtaagacaataaagtgatgcattcctgaagaattggtgttcaagtgggggaactgaaaaataatcagaagttagccagaggaagaggctatggaaaagtttgccagaaagagcaaactgtgtgtataaagaccctgaagtctttgagagcacgatgcttttgaagaactaaaaattgtgtacagttgcactatggagtgtgagctaaactgtaggttagagaggcaggcaggaattacttctgatacaactctaagtatgaatttaccctaggatatttaaaactggggagtgacatgatcagatttgtgtgtttgacagattctctggttgcaggttagtgaatgctttggagtaactgggtcaagactgaaggcaatgaggcagttgtaggaagattttgttataatttagactacagatgacaagtgtctgtacaaggacactggcaatagcagtggagaagaacatggagattctgaaggtatttaggagatagcacttggtaattattgaatgaaagagatgagtgagaatgttccaaagatgttactgaagtttctaatttgggctgtgacatggatgatgatccaatatactgaaaaagggaagagcagcaggttttctggcaaaatgatgagtttcgttggcttgagattcctgtgagacatagaagtaaaaatctagtgatcagttctatgatcagttccatgtatgggtgtggtagacagtagagaggtctgtactagcaaatcagaggtgagtaattgacgtaaccccattcacctccaaggctttgattaccatctctctggggtaaatagatcaaatggccaatggcaaatttctaaggattcctacagttcgagaagtgattagaatagtgctgctcaagtctggctgtaacttaaattctcatgcagagcttttaacagttctggtggcccatctcaaactactagaatctctggggtaagtctatagggcaccagtagtttccagaggtccccagatgattccagtattcatccatgcttgagaaccgctggagaagaaaagaggctgaaaaaaaggctaagagggtgaagccaaagaggtagcaagaaaaatcaggtcaatctgacattaacctgattaaaggaagagagtgttttgagaagaggtgagtagttggcagtagtaaaggttgccaagacatcaaccagggtggtaaattatgaggtctcattagatttagagaattagttcattagtaacctcggtaagacaagctttaatggtgaatccactgcagagggttgagaagtgagaaacaatagcaagcgtcaataacttttaactgggttcttccttcattaatacattttattgattacttatgtattggaaatacacagttgaataaggcatggttaaagcccttgagtaactcagttgagatgatgggagagacaggtaaatggacaattaattatgagataatgtaataagcatgacaatagacaggtacatagtgatgagaagaaatgaatgggagaaggcttcctggaggagtgaaatctgagtagtggacccagttctgactggttttgaagagcacatgtaagggctcaataaatgtgaaataattttcatctttttcacatttgggcaaaatcccctgggatttgctgcacatttcagcctcattccaaagtgtctaggtccgccttgggttccaaagactcatatcaggtagttcagacagtaacacctgctcctgaaatagggagcttggaaatgactgtctttaaaagcagggatttttaattttgagattagctggaaacatcaaattagaatatggaagtggacatacaggtgacaggtctgggctcagtgtgcttgttctcattttacctctgagaatttttaaaaatcatagaaaatcttttttatataaactatgtaaaacactataattcacaaaaatgttcagaaaatagattgtagacttagttctaaaaaccccttttatgggactccttgtgtgcagtggattagacatgttcttttgacattatgccctgtgtagaaaatgctggtcaatgtgcaccatctgtttgtgaatcccttcaaggtctacctcaccacaataccagtctgttttcttgcggaagttttctagaagtcattagactacaatcattttgtctgccagtcattgtcaatatcacattttatcctttaaactctgcccagagaaacgtcctttgatatctgtaactcctcatctcattctgatcctctattggccaggataatatgatacattcctcactggtttttctcatgtatttttaagccttcctatcttcctgacctcctttaattctttcattgctgtctttttttctttttctttttcaaccatcctaccctgtggattccaaaaccaagcatggaactttaatactgaccaccaagcatatttccctggagtattttctgtttacatttttgtattttcttgtttcaaagatagacaaagtggcatccacacattagtgattgtttttcaaatgaatttttaaatccaagagattaattgaaaaccatggaggttcagaattttctttccttattttctaatgttagaggtttaaatatgctcttttaataggttgtttcagaatagtataggtttattcaaaatgtccatagttgaaaataatggaaaagtactaacaactaattgtctggcagcatcagatggtggttgatagtgtgagttttaatcccttccttccccacttacaagtggtgtgaccttgggcaagtgcttagtcatcccttgtttcagattctgcatccataaaattgggatgataatagtattgtctacttcatagggatgttatgaaaagtgagttaatacatgtctttgcatgtattaaaactgtggtacgtagtaagcatgcaataagtattagtaattagagactaactttccctctttaaattatgcttacaaaatgtatcagctggggcacatttttcagctttagttattaaaaagacttgcgtacgttttctgtgggattttgaagaagtcctggcagagaccccaaacctaaaatcaaacccagaagtgacctagttgttagagttctgaggcctcatgatgactcaggatcagcccagatttataaaattgcaaccataaagcttggcactactctctggggacatttacctcttgaactctttctaaatagaatagaataagataaaaaaaaatagctgaaataactggctttttcaggactacaattatataaactgtttctcacataaattgagcatgattattggccattgtgagattttatttaggcaatatgaaaagatgacatttaaacacatttttttaaaaaaacatactttgagaatggtttccaactttgatgttttgctcttttgactcttattctccactagactagctggaggaaaagagttctagaagtttggtacttttatgtcctgtatcacctctaaaacccaaggatattttactttccatcacccaaaattatttttattgtagaattcataaacatcattatcttttagaaacattgccaccaggtttaactagatgtgtgaaaagagtacatagtttaatatagtcaattattatatgaattagtattaatggatttagtaagtattaggacttaaaaggaaaaactaaattaaggggccattttaattaaaaagctaaagactgctgatcaattcttgcagcattgtgagcgccctctgctggctcagttcaaagcaccataatgcacgtaccctgaggagcagaggcattgattatttattgaccacctactaggtgccaggcattcttgcatacacaagcataagttttctgtcatcctcatagccctgcaatggtaaattttgtcatgttctcatatttgaattgattttctcacttttaatatatttttcctaatctgaagttatgtgagtgaagggattatatactttaattattgcttttataaaaaatatcttgaatgaatgactctttataaaatatggaattggaacaagggtcattcatgttttaggattaagaaaaactttagagtggaacaatttgaaatgtatgggttttgtcttacaatggaatagagcttcagcttaaaaaaaaaagacagttggtggaaggtaagggcttttactgcttttctgaaacttcacatcacatcatgagttttgaaagatcaggtaagtatatatgttgtcttagtagccagaaagccagactgagagccccagtcaaatgtctgaattggtggggaagtgacagtacgatgacgcaggtagataaagcaggcatttacttgcattagaaagtcccaagctctcctagccaaaaagtatttcactagcactgtcaaaagttatatagaaagaagaccttagagtctgaccttttccctggtccttctttctttagtggaaatgccaaaatactgagaaccattctaaatggaggaaaagagcagggcaagatagggaacaagtaagtgacctttttcttgaacaagtgaaagtcgattgtacagtctgtgggcatatttgctatattttatatattttcagctaaaagcttcagaaagtggaatagatttcagttaattcatatgtctggaatatttctagagttctatgctatagagtgaaataacttagagatcaaagatgctattttttaattctaatctttccacttacggatttgggcaagttatttgattcctttaagactcaattttctcttctgtagggtaggattaataatacagcaatcctctgctactactgtgggtgtaaaatgagttaatgtgtatgattacagttccccaataatgatatgccacatatattttatgagccaggttatatgctgcattgcatgtgcagcttaatatgcttacattaatctttaatatgatgatgccaatggcaactaacatttgcatgttttcgctgccaggctgtcatccaagcacgtcacacatatctcatttactccacagtccttgcagaaaccctcggagataggcatcttctgaggaccccagctgattctgtcttaagtcatcccactcacaaatcatcctttgtaaagatcttcaaactcagagactgtttcctttcagaccatctgttactgtcttaaaattgaagaaaataatattttggaagagaagtaggggagaatcatagtaaagagaaaagacctctccaaagcttagatctctgagagtttggtgataattatgcagtagtacattctagtaaaggatttttttgtggtgatataatgtatttcaaaattccctccctcatacatactgagttcctgcctactgcatgtaaggcactatcttgagctgtgggaatgctgctgtcaacaaaagtcaatctgccctcatggaggtgacatacaaggtcaaatatctaatctcccatagtccctgtcacacttggagcaatgacactaaaactggtgatatgaataagaatgaaagggggaggagtaaagaatgtatttaactagctttgtagtatttctttgatcaaaattcctgttcatttgcatttatatacacatttatatatgctgatacacatcagtaggaagaaataaactgtagtaagcatgcatttttacattttctttcccaaattaaaaaaaataggaaagagaaaggcttaaaaggagatggtttttaatgtggctttgttttttaaatatctagtggaaaaatgcattcaactgtggaaagaattagtcattctagttgtttaaaagcagggaaaattatcaccatgttaatttctgctgacctcttttatgttaaaccatgtaaagtactttttcaattatggagctgatttgatttactaagagtttagatgagtaatttatgaactgaattgaaaaccagattataaatcacttcagcctgacatttggataaaatataatctgaggttgtttatggtgttctgtcagagaaattcatgagaattagctctcatggcagttctaataatcgtcatttttgaccaacttagaaatattgtatttctacccaaagacaatttgcttaacatttgatatttcaataggtaactaaacaaagggggaacaaaggatattacttttatttttaaaacagtaggtttttaaataatacaagaaaaggcataaccatctgttaatacatcaagcttgtccatgtggtccaggatggctttgaatgctgcccaacaccaatccgtaaacttagaatggcgatcattaaaaagtcaggaaacaacaggtgctggagagattgtggagaaataggaacacttttacactgttgatgggactgtaaactagttcaaccattgtggaagacagtgtggcgattcctcaaggatctagaactagaaaataccatttgacccagccatcccatgactgggtatatacccaaaggattataaatcatgctgctataaagacacattcacatgtatgtttattgcggcactattctcaatagcaaagacttggaaccaacccaaatgtccatcaatgtagactggattaagaaaatgtggcacatatacaccatgaaatactatgcagccataaaaaaggatgagttcatgtcctttgtagggacatggatgaaattggaaatcatcattctcagtaaactatcacaagaacaaaaaaccaaacaccgcatgttctcactcacaggtaggaattgaacaatgagaacacttggacacaggaaggggaacatcacacaccggggcctgtcgtggggtggggggatgggggagggatagcattaggagatatacctaatgtaaatgacgagttaatgggtacaggacaccaacatggcacatgtatacatatgtaacaaacctgcatgttgtgcacatgtaccctggaacttaaagtataataataaaaaaaaaagatataaatgttggcaagaatgtggaaaaatggggaacccttgcccactgttgatgggaaagtaagtataactataatggaaaatagtatggagattcctcaaaaatttaaaaatagaactattacaagactcagcaatctcactgaaggtatatatccaaagaaaataaaatcaatatgtcaaagaggaaaaaaaatatgagattttcttgcaattttttttttagctgatcagttatcattagtgtcagtgtactttacgtatggcccaaaacaattcttccagtatggcccagggaaaccaaatattggacacccctgagctacattgtagagctattgttcacaagcccaaccatatgctacaactacctggggggcttcaaaaaaattcttatgtctggttcttaacccagacatgtcaagtctgagcctctaagagtggggcccaagtggaggtactggtttcaaactcccttcagttgattctaacgtccagtcaaggctgagtactgagtggaaaaggagtggactcaggatcacacaggttaagggagaaaagctttggagaaggccatgatcacaagctgatcatgtagcagtcttacttcttctaccaagattccgcaggcacaacaaaagaaaagagaagctgggcatggtggtgggcacctatatccccagctacataggaggctgaagtgggaggattacaagagcccaggagtttgaggctgcagtgagctatgatcacgccaatgcactgcagcctagatgacagagtgagatcctatctccctaaaaaataaaaagagaaaaagtcccagaaattaagaaaaaacccagaacactaggggaaattacaggatgttctctgaacagattctaacctgtaaccagtacttgaaagttacctcaatattattgctgttgggggctgcctctatggtaaaaacagtgttaagaagtgaaaaggggaaacatatctaaatgtagtatatgtatttgctcacttattaagattggtttttcttctgtggaagaatgattgtttcatgactatttccatgaactttctttctggctatgcattcattctttttacacacattttgaattaagatatatgaaataagcagaagatgtttagtcaaaccataatagaagctttgaattgtttttcgtttactgacctttccatttcagcacccatttctgggctgtttgtcgggggtgggattatgacaaagatacagtcctgggcaagatgaatgaggtcactatttactaagccttggaaataggccagaaacattgtaaataaataaatgtagttaaccttaacaaaaatgaaaatacttattcctacaagatattaatcaatttcaccacaatttcctctgagtgatcgcatcctgtgtgtctaagaacccttgagtgctgatgttgagagtcatctttcagtatctaagagtggattaactaacaactgtataggagatatttgaaaattaatgaatcagaaattttatctatatattagaaggcaactattgtttggaaccacattgtaacagatatgagattacatctagtggcattttgacccccaccccaccaatttcccacttacacacctgccatatgtttttctatttggaatctcttctgctcacgggtgaatgaaccacatcactggcttcctgggaaagctcttcctacatttattcttaagtaaacatctctttcttgtggcttctggagggctaattgttctcctatttctaagaatcattatttttagcctagaacaagaagaagggaagggcatgagccacactggaaataaagtcccaggactttggtgagactacatgggacacatcttaaaatagcctaatgatttagtggcccttttcccagagagctgagaggcactcatgtggagagaaagaagtttcagggaagtgatgttagtaagtgagaaagtgagtagtttgtggtgtgtgtaaacctcagtgtgagcctgggcagtgatgacattgtgatgttacctgatggagctaaaactaaaacaagaaggaggcaatgcagaatattaaatagataagagttcataatgcaagagtttgtctagtcttcacttagaaatttacaaaacagttcagctaagaaaccgataacaatacactatttttgcacataattttttggccattttcattaagctatttacatggtacattgtggttctaaactattgtctttttaatcttctgggtggctctctctagaataatagatttgtttctaaatattggatgtaatcgtaatgaaagtgtctattatacacgccatattatttgtgacccccatcctctcctgtccttaattttcttcttgctcaattgtcttaactgagtgttgactataaattcataaaaattttagtattttcagagggaacttattaacatggctattgtagtaaataacaggaacttcatgtagactgaatctaccatttgttatctatttttgacagtaaacaattgattttagtgaaaactgaaaattgtattataatgaaaattaaaatgccttgtttgtcattctcttttaatttaaattttgttggtttggcattaggtagccacaataacaactccctattttagaccttacctatctcccacacacaattttttttctcctaccatcttccctctgttttccccttctcttttcgtgtcatttctctcttcatcccttcctttctcaagttaacacggagaccttatgtgtccacctggactgttctttaccaaggtttgggccacagcaagactcctttccaattctaatagcaaccattgccaatcaaccccagcattctttgtccagacctagtcttagtcctcctcccttcagtctagttctccaggaagccattaccagttagtgggggttgccatttattctatgtgtatgctgtcgagaaaaacattctctgcatttcacagtaacagtgttcctgaccatgagaaccacttagggcaggggtccccaatccctgggctgtggatgggtactggcccgtggcctggtaggaactgggctgcacagcaggaggtgagcagtggaccagtgagcattcccgcctgagctttgcctctgtcagatcagtggtggcattagattctcgtaggagtgcaaaccctattacgaactgcacagccgtgggatctaggttgtgctttccttagcagaatctaatgccagatgatctgaggtggaacagtttcattccaaaccttactactgcccatggaaaaaattgtcttcacgaaaccagtccttggtgccaaaaagattgggaaccactgacctagggtaatcagggcctcacctggatctgctggagcagaatctccagaggaggagctcaaacattttcttcacttcagttgtgattaaaggttttgtttttcctgaatgacattttagaaaacgaaagtataaacaaacatcttcccacactccttaaacaaaatctctcctacccaggagacttagtgcaaataatttcacttctcaaatattagccttaccaggcatagtggcttatgctggtaatcccagcactttaggaggtcaaagcaagaggatcacttgaggcaagagttcaagaccagtatgggtaacatagtgagacccccattggtaccaaaataaaaatattagtttgtatctctgtaagtgttctgagtttgcccaggaagctacatttaggggtcaacattataggaggagacaaagtgccaatgatatagagaatggtcaccataaattcatgaaaacttggtacctagtgactagtgttcaaaattaacaaaccgaagttccacatccttatccaatcaaattgatgctaatgtatttatctgccaggaattaagaacagatgcaatagaattgtcatatgccaactgaagagggtgaaaagaaagacagcaaaaagaatattaggatactgcatcttactcttccccaaagtaacttatttaaaattacttaacatgggattttaataggaacccagctataacacaaataattttttctaacaaaagaaaataatgatcactgtgaagaccacttcttatatagtatagaagtactatctagtacatgagcattttatttatgtttggctattcatgcatttgccaaagtatctattgaagttttttgtttactactgagataatctggtttccaaatcacttttatctctgttgaaaacatgagtcatattccaataaatgtaaacatagggcagtttcttataaacgcaaggcattattggtatgctaattgtatgttccactttctgcatgcttaatcatcaccttccagctccaataaattgtaccttaattgctgaggatgtattatagtctttctattcttaatatctcagtagaagaaacatttttaaaagtgctctgataatattgcttacgttgttagataaatggctaattcttttccaaacgttagacacagcagttatagttgcacaacctggtattagctttaagtggttttccccactcctagtcctgttagctatctcttatatgattgagtctatctttttatggtgctaccagtcagaggtcatttctacaaccaaagtcaccttgtattagagaataattagttggcacagagtaattcacatttatcctcacattgtgcactgaatataagatagagaagtagttaacagaaaaatactggggatgagtgcacagttttctcttcttttgtagaatgttttcaatacaactgataaaattattttcacatgctataggtagccctaatggagaaccttgtggaaagacttgtttggaagagagagacaatcagtggttgggggtcacactttccagacagccaggagaaaatggatccatcgtggtaggtattggaactggtccacagatccatcgtgaaatcagctatcctgggtgcagctttcacatcatttggtctacttttattttattcagacttgtgggcatagatggaaaaatatattttacataaagaatgaaaataagctccccactggtggttgctatggagtgccccctgatttacgaacagaactgagtaaaagaatagctccgtgttatcaaggtaaggcatgattttgatacgaattagataaagataagtaagtgaataccttttagtgttttaaatttatgttcaagtttagatgttcagaggagagggagatcttctaagtaattatgttctttttaactactcaatttctttctttacccaattgcaatagcattatttttcataaatgcactttgtgctataaaaattctacaatttgaataactctgaaatctagctctttaactacccacttttgcatcaaatttactttcaatttggtataggtgagactagtacccagcaaatataggacatttaaattgtgaaattatattggaagtctgattaaaaagaaagactttaggtttccttttttacctttagcttccaggagtaattgggaagaataatcagtgtaagcaaatctttcattcgcactcactatctctttttctaatttacatgttttccttcaagcagcaagagattcgtgtcttgagtttgaaacatttttctcatggtaactctatgctataggtagcatcagcaagtacagagctaggacataacagaagtgagcagaattggggtgaatgtcaacagagcatgtcagaggatatctgcagcaaaactaaaatccaacaatgcgtctttaggagctaagaaacatatgaatgcaaattcataattttctaaccaccctcactcaaaaaaaatccctcagcacgcaaagaaaaattttattggttggcaaaactttggccaagtatttgagttcaattatgggtgctaataagactttggtctttcttccttggaatgtgatgtcacagagcagcttatattacatatattcctattttgccttcggatacccaaaagataaaaaagtgaatattgattctcagttgcaataaaactcagtcttgatttctgtatgtttgctgtcttctttttcctaatagttttcggtttatctttaaaatattttcatagtagagtttttagtttttctcggatatacatagccacatactacaaaaatctcaagatgccataaacctaacccatctacaattgtaaaatacaggatcaggtaagatatatttttttaaagggatgaaaactcctgaccagaaatggtcattataaatgacttaccaatttattgaagttttctgcttagcaaagccaaaaggaagctgtggttccaattcttattttcatagaagtttacttatatcattttttggaagaagcaaagctttgcccccttagaaagttccaaaagcattcattcatgggactttgtaataatagacctttatgtccatgcagatcttgattcagtctatatttcagttgggcaaaatctaagaaaggacactctcttcaattaatggtgttagcaatagcaaagacatggaatcaacctaaatgcacatcaatgttggattggataaagaaactgtgatacatacacactgtagaatactacacaggcataaaaaataacgagattatgtcctttgcagcaacttatatggagctggaggccaataacctaagcgaactaacacaggaacagaaaaccaaataccacatgttctcacttataagtgggagctaaacattgagtacatatggacacaagggacacaaagtaggcacaaacactagggcctacctggtggtagacggtgggagcagagagaggattgaaaaactacctatcaggtactatgcctattacctgcatggggaaataatctgtacatcaaacccctacaacacgcaatttatctatgcaacaaacttgcacatgaaccctgaacctgaaagtttttttttaaatgcctttgggaaaattggctatctgcgtgcaaaagaatgaaattgaacgcttatcttagcaaatataccaaagtcaactaaaatggattaaagacttaaacacgatacctgaaaccataaaatttctagtggaaaacataagagaaaagctacttgacattagtctaggcaatgagtttttggaaattacaccaaaagctcaaacaacaaaagcaaaaataaacaaatgagactatattagaaagctcctgcatagcaagggagataatcaacagagtgaagagacagtctgcagaatgggaaataatattcgcataagtcctatatatttaaaatgtataaggatctcaccaactcgatagcaaaaaaccaaataactgattaaaaattagtcaagagaccaaatggctttctttccaaagaaggcatcaaaatggccaataagtagatgaaaaggtgcttaacatcaccaatcatcaaggaaatgcaaattacaaaccactatgaaatatcacttcacacctattagaattgtcattatcaaaaaggcaagagataacaagtgttggcgaaggtgtgaagaaaagggaacccttgcacactgttggctggaatgtaaattggtacagccattatagaaaacagtagagaagttactcaaattaaaaataaaactaccatatggtccagcaatccctcttctgggtatatatccaaaggaaatgaaatcagtaccacataaagagatctgcgtttttatgttcattgcagcagcattcacaatagccaagttacagaaacaagcttagtgtccattgacagatgaatgaatggataaggaattgtgtgaggtgtatatatacacacatatatacataaacatatacaatggagtattattcagcctttaaaaaaggaaattctgacttttgtgacaacatggatgaccccgaaggacattattttaagtgaaataagctaggcccagaaagacaaatactgtatgatctcacctatctgtggaatctaaaaaagtcaaatacatagagaagaaggtgagcagagagagggttaggaataggaagatgttggtgaaagatgttggcatttgtgtaggatgaataagtctagagatctaatgtacagcatgaggactggagttaataattgtgcactggaaatttgccaagagagtagatttcactctaaatgcacaaaaaagatgaccgtgaggaggtatgtaaagtgtagtaactgtttcactatgtatacgcatatcaaacctgatgttgtacaccttagatatacacagtaaaaagaaagcatgggctcaaaatcacaaatcctacctctgtcttactcatttgacaaaaattttttgagtatatgctgtgtgtgcttcctatgaagaagcagagggagatatttttattgccatttcatagggatattttgaatactaatatggaagggatttgttatatcaaggttactggtttttatttttaaaaaatacaaactatagaccttaaaggacatttttgtaaggatttaagaaacaaattatggagattttatctttaagataaaattctgagttgttttaatatttcattttagattatgtgaaaaaatttggagaaaattttgcatcatgtcaagctggaatatccagtttttacacaaaggtaattgttcaaaaaatagctgctataaatgtttacatatagaatcttaattcttctcatggtttggaagactgatatgttttaaagtaaaaattgtgtaacatcttcatcatgtctcttaaacttgagtattacaacttaatttttgccactcaaaattccacatatatttcaataaaatatcaatatttaggtggctatactgcataagatgaatgaaaatctatactaaagaacatttctttcatcaacaatttaatgaaaatcaaaagtagctatggttaattgtcttgttaacttaggtattgaaaatatcaacccaagtatgttattttaaatttctagttgaggggcaaaggaagaatatcagtctatatttttcattaaattatgataattgggaaatttaagaatttaatatccccttaaatcagatagacaatattcccaagatttcttactctctttctaacattctaggatatgtatgaaagggcacagttgtactcaaccccaaatctctcagttttcttaaaaacacactgtgaattcaggaatgttttttgactccatgaagatcaggcactatatcaagtttggggttgacagtgtctgcacatattaacagttatattaaaatgcttacaaagaaaatgctttcttcagtttaagttgactttttttaagtttaggattgtttaggagtatagaaatatgctaaagatatttaattgctgaaagtgttcatactataccaggcaagggcatgctcattccaatcttctcctaaattttcctctgtataacaaaatatgccccacattccattggacttttttccctaagctaacattattagaaatgttttccctaagctaacattattagtcaggggttctttgcaaatagaagtaaattaagtctactttttattctttggcaacacatgattatctccataattagcactaatgaattacccacatacgattttctttaataggaaccacattgcctttctcccaaaagcttactctagcgttcagtacttgtcccttcaacttttgaaatccactgataaatgcattgaatgagagtttcagagtttcttctctgagatttcgcttttagaaatccattagccaacttctctggctttttttttttcctagtatgttttaacttcacagaatattcctacttcaggaattgattattataaaaatatgttctcttcactatcgtgtatctggaggaggtttggggtggtgagattaaagtttaaataataatagttttttttttcttacaggatttaattgtgatgggggccccaggatcatcttactggactggctctctttttgtctacaatataactacaaataaatacaaggcttttttagacaaacaaaatcaagtaaaatttggaagttatttaggtactataaaaattgacaaacttaaatgatctgtgccttacaaatactagtactgtaattataaataaggaaaatgtattttccaaagatctaaatggccagtataattacagtaataattaggcagttcagaaactgtctcttaaggataggatttatgaaacttcagtgaactcagtattttacatttacacatgtgactaaaatcatcaatatataattcttaatagaactcctagtttagtttactctttgtagttcactttaggaaacactagtaagtttctatctctatgtagtcaactgattatacaggttgtttctgttgagtgcctcccatatctcaggaactttttgtgagaactttacatgaattaactcatttaaacatctgcataagtaagtatagtttccttaagcaccaactgaaagccagtggtactgtccaccacattctggatgaggggctccatgaagcacatgggtctaaaaacagctcattgaccttcacctcataagttcttagaatatttcccagagcttaactatttttatcatcttcctcctcctcactgtcatctcataagtcaatagtaggatactttatcatctgtatttcaacagaaatgcaaacttttctggcattaatgtccattacaagaaaataaatgcttttcctatagattttcagaaaatccagctttgtttaaactgtatgtgtgtcaaatgtgagcatcttacactcagaaataattataaaataagcactctttctttctattcaaacagaagaaatttcaagtagaagaatttgttacaattcaatgttttcttgtttatgatgaaaggatactttgaaatcgttcaagaaatataacttgttacgccttttatcacataaaatacgaacaattttttgtcatcgaaaagtcatcacaaagatttggtggcaaaaaaagttgaacttcagcaagatgtttatttaaagtttaaagttttcatttcccaattgagattttatatttaatatactctaaaatatgtctgttgtaatcctagaaactgattatactgcaccagatgtaaagatgctattttcaatttctaggactcggaaatacataccatatgactgcagttttgtaaaacatatcaaaattgcataaaacataatgaatgaccaattttgagtatctctatgctaaatattcttaactgcttaatgtagtgattttgaattcggtatcatttaattttatgatctattttacccatattaccatatgatgtactttacccaggactctcatactttctcccttttcttaaaggatattcagtcggagctggtcattttcggagccagcatactaccgaagtagtcggaggagctcctcaacatgagcagattggtaaggtaagaattacatttttatatttatttcttcacaaaggttcaaatatattgcatgaataagatattaaaggagaaactgtgaatgttgtaaggaaagaaagattagaaatgatgaacagattattacatttagtggaattggtgaaagatgttatggttttagaagaaatttagcaaaaaactttctgaaaaattacaaaagtcaaacgagacattgttttaagaaaattataagatagaactatctaaaaatgtccagggagtatatggtgaacagtttcacagctgaaaagaagttgaggtagctacatattttgtagtaagactagaaagaaggaaatattattcctggttttagtttttgctgtgtcgctatgtgaacattggtcacccagcttttctgagccttacttttgaaaactgaggcaaacaacacttccggtgccccctctataggatcaataagataattcctgagaaaatacttttgcaagtaaggcatattaatgtaacagtggatcccaaacccttttgtgtcactctcaacaggatttatttttatatttgagcaattaactgcaagttgtaaaaattcatgttttgatcaaacagaaaggagtggtgttttaaaaaatgttattcctaaaaacttttctaattctttccctaattacaggcatatatattcagcattgatgaaaaagaactaaatatcttacatgaaatgaaaggtaaaaaggtaatatgtctctacctttagtatctctgtgggcttttgcgagtaaccctgcttttttctcaatgagtggatctggtttgttttgggacagcttggatcgtactttggagcttctgtctgtgctgtggacctcaatgcagatggcttctcagatctgctcgtgggagcacccatgcagagcaccatcagagaggaaggaagagtgtttgtgtacatcaactctggctcggtatgtccaagtgccccaactggaagccatttatggaattatgatcaaaactcaaattggtcttattccagagatctgagattgttttcaggtttcttttattgacaaaagttaaaattctaatactgtactgatgctctaaaaattaaatggaatatgatgatgaatgggaaagtttcccattttgatattccagaacaatctatgaaatagattgttagggagtttctctgttcacagcttgtatgtgttcactctggtagctgaaatttctttcagttttatgttgatactaatctaatttgaaagaataattttcatacctccttcaaaatgtagatttcttaatagttctagttaaggtccttatataaattataaaatattactaggactagaagatgaagaattaagtattttcaatttcttaatttgaaaacaattagaaacaagatgtgttttgttaaaacattaattttcataattcatatatattatgctcttgataagaatataaaattggcaaggtttattccacagtggatatattagttgcataaataggtaattactgactgtgattcaattttaaattattagtgataatttgtggtagaaactgaccacttgataattcaaagacttctggccttatcttgccagcctagattcttggcgttaagtcaaaacaaaaaattggtggagtcttaatgaaatcttcttggcccgttctttgatttgaaaacaagagtgggcttgtgagaagaccagttcatttataatggcatagcatttttcagtcaggattcaacaaagtggactctgtttcaagaaagtatttgtaactatgttcagtcaatggtttcagagcatttagcaagttcttatattaagcatcagactaaactctacaatttaagaaagaaaacatctagtctcataaatggaagattctctaagtttgcaatatagagcagggaagttatttatgctttggtatttcctacactgtatttaacctacatttaatagtaaaagcaaattatctacacagtgatcattaaatctcatagtagtaatatcagataattttcttccatttctatctccaaaaatagtggtctttgtacttgctacatcctcgtgctgcaaatttctcttattttcctctttacgactaaacatccacatgtatacctaaagtaaagcaatatagaatcattttcccccattgaaatcttaatctcattaagaaaaaacagataaacccttctacccacttggctaccctccttttcttccctcatgcctaccctgaggcactactcttatgaaacacttggattaaattaataattgattccagcttttaaatcctctgatgtagattagtcttcctttgttccatctaacctctcatttctcaactctgaaatggcactaaccactaagtggcatttatttacattgatttgatttggtattgtgtgttcttctactgtagtatagatgccttgtggagagagattgtctgtcttgtttaccgtggtttcatcagggcttggaagagtcctagacaagtaggtgaaattgtttttgaatgaactcaagaaagaatgatacttgagtgactggcttctgcctacttgtactaaaatagctctcaggggttcctagcgaactctcagtcaccactctctgtgactagggaatacttgaagaagagtgatcacctcttcctattgaaactctccttccagaacttcagagccattgcattgtcttaagtctataccttgtctgttctccctggagccttttcacccttccactccttaatttggacatttgcccaggagccaagctgttttatccctgactcctttgaatcaatcattcactgcttcagtgtcagtgattatatttataaagatgattcccagtcatcttagttcatttaaaataaaataaatgacatttagtgagtatctgaaatgtaaccaggactataaaaagagctaataatcttagagctgaccccaggacttcaggatctttgtgaagacaccaatgggagtaaacaattgactacaacatggtacatggcacattataagtacatacaaagtggaagaagtgattccctttgggtcaagaagcttttacagaggaagtagtgtttgagaaagacaaaaatcagaatttttctattggaaaattcacaagtgaagacacttgacaaagagcatgtgtatgcttaagaggtgccaagtaaatttgagaagaataaaggacatggcctttttttctttttaatacaaaagactgacatgatcaaagtttcagaagagaactggtagctgttattgcaacagcccattattacattattgaatgtcttattgtagtaactcatcttcctccttcctagttatcagttgactaatctatgctccactgctgctggttccaaaggccagcttcaatcatgtcactcctcaactcagaaaccttcagtgtttagtattattccctaaattaagagtaaattttcctcgacaatcaagacgctacactataagactgcaatctgacatttttgtctcattcattatacataccattcattagagccaaattctttggaatttgttttgctgctagttttctctgtatatcaaaattatgcttatccttcaatagcctcctggcatgccatttcctttatgaaatgtcttctgttataattagattttttttttttgcgtctcgatattgtaaagcagtttctacttctcagtcatttatatgcttgtgctaagccttccctataactgtgggcttattgaagaaagggaatgtgttttgtgcacacactgccttgccccttttcaccttttcgctctttgcacagtgtgttgtctttagtagaggctcaataaatatttgctaaattgaattatcaaaatgatttattggattttcaaagctagagatcatgagtttgacaattttttatgtcctctgaggtctaatcatactattatgtgttgacagactcaataaatatttattgaattgaactgagatgtgtgtactaaagctcagtctgtagtttgtccaacttgtttgacacattagaaaaaatccatatccaattacaacagtaaatcgtgtaaagttgtcagggagtgttataatgacacgttttctctccctttctatctagggagcagtaatgaatgcaatggaaacaaacctcgttggaagtgacaaatatgctgcaagatttggggaatctatagttaatcttggcgacattgacaatgatggctttgaaggtaattaaaattatcaaattggtacttgatttctgcttttaaaatggtttatggaagaaaatatgattaaagttttgtattgttttccttcctatagaagatggagccagaatggcatgctaagttttttcttttctttagtgttatatatgacttctcctcaattgtcacccattgatctttaccactgttaataatggatgatattcaaaataccttatttcagtgattctaaggcaccattgattagaaactgcattattatttatgtgtccctaaaagctacctattaagctgttacacccaccatttttctgttaaggaagatcctgatttcagaaataataaaatatgggcaggaaatgtgttatcatagactcattggaccacggtatttaactatcagtacaataaagacactgaccaaacccaacagaggctggctgcagagctggagcagggttctggaagcccctgctctgccaggcattccctatagctatgttgtggaggatctggggaattcaagaagagattctgagcaggccactgggaggcctggggacagtggctgtttacccactgatattgacgttttttattaattgaatagatggtagtgtggtaactgtgcatattgccatggatgtcagtcctgttgttgaaatccctgcatctactagcatttactgttaaaaatttgcttttatcaatctagttctaagtggtaagtaactgagtctgttaaaactgtgcataaaatggtgaataggtttaagaggagagtgtgcagtttcatggagttcagcacataagtatttgatacttatttttagggttcataattgtcttcttgataaggccattggcaattagtatacggtagttttaggctaaagctttctcagagattaaagtgctggggtactataaattctcatctccttaatgtcttcttgtacctagataccacaatctactttaaatatataaaatgcattattctacataaaaagtcagttattgaatacatgcttccagtgaaccttcttaaacgtaaaatagtgtaacttgttatacaagtattactgttctggattttttgtttactattttaaaactgaaagaaaaaaataggtgtatatacttttggttattacgattacacataatttttttaaaaaacatgtatgaatgggagcctctctgtaccccacccatcactagcttccccctacttcccttaccctcccaggaccccaggttgggcattggctcattttcataaataacctcatcccagacaggggtgagggaaggtgttgtggctgttttgtttaaattggagctgtaagagggatcatgtcatattttatgcccttcctagaaaggggacgggacacctggtttgcattcctgtattgaccactgctgccgtcactcgtcacattgagttcacatctcctgagagtttgtataaattcaggtcagcgctgctcctatgcagccatccagtgtcatagaaaagcaattcactgataactgatctctccattcagaagtgtgcagttttaatgtatagcaataagaaattgttatttatctattacaagcctggtttgattaaaaacaaaaaataaaatgttattatgcttgatgttacatctcataagtaaccagcaattctgtgatatttattgaacatttattttgggtcagatgtaccataccaggtacagaatatgtgccataataaatgaaaatgaagaaaatagtggcacccttttttatagctctaccatcaagtagaaaataaaattaatacttgaaaacataagccttatttcgtggcaagtttaagggttctacctaataaagacctatggataaaaccatgtttatgcattttgatttgtcatgtgtgttgtatgagacaacccaattctttcttttaaaaacacaaaatcactgtttagtaaaacacttattttgtctatgatttctctgtcttgttttaattgacacatttaaagaagaccattcactatttttactcttttgaatgtgaaaagtatatgtatgtacacatgagaaattatttttgtggcatcaaaatgtcaatgacaaatttgaattattgttagagctgtaaaactgttgtgagcataggcaaattcccttatcaaaattactctatacctgggaaaagcccaagtaattttccagtggtgtgaaggacagagagtgttcggtcgatgagacttaggaagaggacttcggaggtgactttcaggggacagtgcagtctctcaatttgtatatgagaccctgggcttcacacagctcttgtttcactgaaatccctcagcactccactcctaaaggtaatacctttatgtctggaaggttattatgcaaatgattttttaaaatattcctgcatatttttgtgctatttataatgtcatttataattcaccgcatatttacatttagtggctacattaacacttcagtgagtgtttataatgccatatatctttttctacttggtccagttctttttttattttattttttttttgagatggagtctcgctgtgtcacccaggccagagtgcagtggcatgatctgggctcactgcaagctctgactcccgggttcatgccattctcctgcctcagcctcccgagtagctgggactacaggcacccgccaccaccccagctaatttttttgtgtttttagtagagacagggtttcactgtgttaaccaggatggtctctatctcctgaccttgtgatctgcccgccttggcctccaaaaatgctgggattacaggcatgagccactgcacccagccctccagttctaattttatcttctattttgcccatgagtaggcgttttctatattttagatattagcaagaccattgctgatgtggaaacagggccagtgtgtcctagttcttggctgaggtcttttattccattccaccatgtctgcctgcatgaggcagcaggaatttacaagttatttttctttatgtgtttctttattactttgccccatcttctcttattttatttttctggggtggatgggtaaagatactttgtgttcatggcaagaagtgtaggattatgaagaaagagactccaacattggatgcaaatcacacttaatgtctttttattatgcaaatttttgtggtactttgcatagcaaacctaagttatcttggcatttgtcatatcacttttcaattgttctcaaatggaaaaaaaatcagactctatttcctgtgtccttagaaaagcaacacaagccattttctaaaaagatgaccaattgatttggcaaaaatttagtgtaaaactcatcaaaagcatgaaaaaatcggtcagacagactaatgtacctgacatgaccaacagcagatcacattttttatttcatatgtaaaatagacttgaccactatttatcggtttagtttcttttttcaccttgagccttattatctgacttttagactatatgtctgaatatttttagagcagaaccataacaagttctacaattgtaaattacaagctgtactattattttgtgtgcacctgaaatattaatatatcgtcaaatcattaagtgagcacttctgttttatttgtttatagggtacaagtgaaattttgttacatgcatcgattacatagcgatcaagtcagagcttgtgggtatccatcatccaaataacatacatttttacccattaagtaatttctcatcatccaccccacacaacctctttcccttctgagtcttcattgtctatcaaaatgtagcaggacgagccacagacaaaactcttcggacgccgagttaaagaaggaaggggtttatttggtggggggcatcggcaagactcctgtctcaagagccgagctccccaagtgagcaattctgtcccttttaagggctcacaactctaagggggtgcacgtgagagggtcgtgattgattgagcaagcagggggtacgtgactgggggctgcatgcaccggtaattagatcggaacaaaacaggatagggattttcacagtgcttttctatacaatgtctgtaatctatagataacataaccaattaggtcggggtcgatctataactaccaggcccaggatgtggggccaggctgtctgcttgtggatttcatttctgccttttagtttttactttttctttctttggaggcagaaattgggcataagtcaatatgaggggtggtctcctcctttaattctactttatacatccatgtgaacacatttttaaagcactgatttatgagtaagagcatgtgttattttactttctgtgcctggcttgtttcatttaagataatgacatcccccaccagttccatccctgttgctgcagaatacatgatttcattttttttttttatggctgaatagtattcgtgtgtgtgtgtgtgtgtgtgtgtgtgtgtgtgtgtgtgtgtctgtgtgtgtgagagagaacctctttgacacagaacaggaattggtattaggaagggaaatttgggggtatgacctctgttgttaatgagtctatagagcatgggaatagcacaggatctggaccaggctgcagggtttaaatcccaggtccacaacatcctggctctgtcaccttgagcaggttataccttgtgcctcagtttcctcatctgtaaagttggagtcagagtatttatttaatatggtggttgtgagagttacttgggtaaatctgcacaaagtccatatcacagtgcctacattgtaaacatagtataagtgttggctgacatactaataattttgatttatactaaaagaatgaaataaaatgttcagataagaagtaaaagcaatcattaaatacctatgttctgtatattgtgtcccaaattaagttgcataggcttggaaaacatttattgtatatatttcaaaacatatctttctcatttgaataatgatagctagaactttattatataacagatccaagcgtaatccaggaagagtctgaatctatatatgtgatagccagagattgaatatacccaagtgtcctctgtatggaaaaaaatttatctgggcctttttaatattttggctgaaaaggctcatctgtatttttcctaaatatttgccttaatctagatcactcttgctaataacaggtactgtaagaattacatcgccgggcgcggtggctcacgcctgtaatcccagcactttgggaggccgagacgggcggatcacgaggtcaggagatcgagaccatcctggctaacacggtgaaaccccgtctctactaaaaatacaaaaattagccgggcatggtggcgcgcgcctgtagtcccagctacaagggaggctgaggcaggagaatggcgtgaacccgggaggcggagcttgcagtgagtcgagatcgcgccactgcactccagcctgggcgacagagctaaactccgtctcaaaaaaaaaaaaaaaaaaaaaaaaaagaattacatcaaccacttcactaccaaggcttgttatttttttcatgtgttatgaagaatcaatatataggaaattctagagatgtaaccacagtggtggaagtataaacccagccctgattaaaataaattgctgtcttcccttcaatggctataaaatacttttccccactccttcttctcacatccctcttctcctcctcctcctctttctcttccttaccattcctgctcctctgttcctgtatgtatatattctgcacaaatctgactccagttattccaaaccaattgccattcttcacatgtgccatgctgcctattgcttccctgcccttgcacaggcatttccctgcctctaaattgcatttccccttttcttcacctgtctatgacctatggatactttaatctcatcttgggcatcattttctatgagaagccttttctaatttccaccacttcctcctcatatgctgtgttattagtctattcttacttgccattcctccccactagacaatgagtttcctattgtgatactgccctgttttgccagctcctggcaattgcctggaatatagtaggtgctcactagctgtccgatgaatgaataaatgagtgggcatatttgtatatgaaagtttgaaaaactttgtacaagtcaaaagaggctagcctttgtatgtctaaacaaagtaaatataggtgtctaccatttctttttaactgaaaatagaaaagcttttgcatttttaaaattttacattgtttagcatggagttatatatcaaagatatttttatcctcattgaaatagtagcccaaaattaccactatagttgataaaatgattttcaatttgatggtaataagtaatatcatgaagacaaactactttaggtttctattaaataaaccattgcatataagtaaatatatgcataaatagacaatacttggtaaaataatgaaatgctcttgcaactgtagaatattatttttctcctttgtgctatatatttatagcatttaagcaaccatgtcatttactcaggttttcttatattagttgatcagatcttttttcccttggtatcatagtctgacataattatcaataatacagaaaggtgataaaatcattattttacagaacaaatcctttttctgaaatcctgtgtgtctgtgtgtgcagatatctctaacagataccttaatcattttcattggaataggaattaaaagtcattttgcaaataaggtaagtctagactcccaaaataaaaattcttatgatcaaatattcatagattatgaggttattacatgcgaggcactgctctaagcactttacataggtcatatccttttatgctgagagtggtttttattatgatcccattatataggtgagggaattgaaacacagattaagaaatgggcccaacctagtgtggcagtgcccagtggcagagctcggatcagaccccagtggtctggctgtagagcccgcgctctaatcattttgttacactgcctctcagatgcttccactctgctttcttccatgtgcaaactattgtgtgcaatcctgtatatgatgatgatagtattttatttttttctgtctgagaagactgttatttttcataaaagagagttttcttatgtgatatgaattaaggttcataaggttagttttcgaagttgcattctttgtatcaagaatttatttttccattgtttaaattattggatagatgttgctatcggagctccacaagaagatgacttgcaaggtgctatttatatttacaatggccgtgcagatgggatctcgtcaaccttctcacaggtaaggtactattctatttccaaaagaagcattggttataatgcatatccttaaaacttccagggtttatgtggacttatttttcttaattttctcattcaaatacttaacactttatttcaccgtttacttatagtggcaaatgatcttattttaagggattaataactcttcaaactccctttctccataaataagagttgtagttcatttccaccctagcttgtctatagcccattaaagatttctctttgtatttcattaaattgcattctgaggtaatatccatatgattctaaaatatgcccatttggatttgattaactgtgtcttggccttgtggctagagccccatattttgtgaagattgcacagggagatcaaacacacatatgattctctgggtttttttcactgttgagtagtaagatgatagcactttaataatgtctcaggatcttactctgacttcacttttgtatgttgaaaaatcatggcatttcagtgcctagaaaaaaaatgacattcatatgcagagaagaaaagtttttcccttgatatttgcatggaaatattagctggcacagttatttttgattagaaaaatgagcaaatggttctctgtaattgacataaaagaaatgtatttgtctagtaaaaaaactaattttcttagcacgaatatcagcttaatatttataaacctgtatgtgaacatgaaattagatatgtggaatagaaagtacatatctggctagggttgttcttgttaaaaagtcagcttttatcaaaagcctcagtcataggctccactaagtgattatagacatcttgttttacaaattcttatttagttattcatgttgggcaaggaagtaaatagagagcctgaagagacctaaggaggccagacatggtggtacacacctgtaatctcagctactcaggaggctgagctgggaagactgcttgaacccaggagttcaaggccagcccaggtgccatagtgagacccccacctctaaaagacttgagagggagtaaagactatttcagaaaatctgtacagcataattttccctttgctcgcaaagtaaatttttttagtcacaaatgtcacctgttcatttcaagctggaaaaattttgtcacccttagaagtatttgatttaactaaaatgcagctttgttagtagatgtgccaaggcatgcctgggaaagctcattatttagctaaatcattgcttctctggtatattagtattcaaaaactacttcccactcaactttcctatttttcagagaattgaaggacttcagatcagcaaatcgttaagtatgtttggacagtctatatcaggacaaattgatgcagataataatggctatgtaggtgagtaattagtttatcataatttataaattggaataagctctatcatagatactgctttatagtgaagtacgtaaaactggtatgaaagacttcatcttactgataattttttttcttcatttttaaagttctccttaattcattcctaaaatgatcaagtaattcctcagcttaacagtgctagttacacaatgttatagtctgtgtttttgaaaacatgaaagcactcatgaaaatcaaatatatgaggaaaagaaagaaaatttttatttagaaaaatttgagtgaatgtttctagtccaaaaattgaattttgttttagtaaacttgtttttgtcataaagtttatgatatatcataatgggatgaatgttgtacatgaatagattcagagaaaagtggaaagaatcgtaagatgttagcatatattctctaattttctataaatagtgttttaggtagtcaaaggtgatacgtagttaagtatttatggctgaaaaataattctctttgactaatgatgatcattaatctgtgttgttttttatcctccagatgtagcagttggtgcttttcggtctgattctgctgtcttgctaaggtaagactgatatatttcactgcttaattgcaatttggtttaattgtaaaatgatgggaggtggtgtttaaaatcagcagtggtagtgacctcatgatgctcttttggtattctgaagcttaattattgatttttagggtatttttttcaccttacagagatataattaaatcatcaaagtcaatatttttagacatttaaataaaaagttattttgccctgtgcacagaaatgtaattagtatgtacacacataataagactcatgaaattacttggtgaatgtaaactgaaaaaacaaacgcatttctctccttaaggaaaaataattctgcaattaacattgctacttttatttccttctcaggacaagacctgtagtaattgttgacgcttctttaagccaccctgagtcagtaaatagaacgaaatttgactgtgttgaaaatggatggccttctgtgtgcatagatctaacactttgtttctcatataagggcaaggaagttccaggttacattggtgggtatgccctacaatattaatgcttgatggggtgcggttcattcattaatcccacaatcctgcttggagccctcaccggttttcaccacggagatcttctttagagcgggggagagaagctacctgatgcatgcttttcctctccatcttccagattcttgcgttgttctaaccaatgagttgggcacattagaaggctgtagttgtttagagattatgagaagcaaaggacatagtatcacttgctatattagtttaaggtaaaccaactgagcttaacctgtgaaatctctttcttcttgacattggataaaattgacattttatcaagtgtcaataattgaggaaagccaggcatggtagctcatgtctataatcccagcacttgaggaggctgaggcttcagcccaggaggtccaggctacagcaagctgtcatcacaccactgcattccagcccgggcaatggaatgagaccctgtctcaaaaaaataataaccataaaaataaatattaaaaaaattgaagaagctagacttgattctgatttctataaataatatgcttgttaataatttactgcgactacaagctcctaagaaccgtcacctatgtccctctgtataggcaactctcaattatctgctctggagaaagtatcattgatatagctcatccaaaatattgtggtggggaatcccaaatcatgtatatatgactttataagtatttttgctagcagattaatgttcctaattatgttttatttaggctaatataagaataactactagtgaaatgagcctgtggtaggagaagagcatatctgggcatatcatttagagattaataattcacaacgtttatgatcccctattaggacctgtcaagagtttattgtattcatgaccctgcaggctgctgttatctaaacatcgtgaagggcccacatacttattaaataatgccatggatatgtaatagaggtcttagtatgcattaggtacacaatgagcctgtacctgaaaggtacacaatgagcctggcatctctactttagatcagactcatttttaatagtgtcctggaaaagtcatggccagagctacagtaataagagggtggacaagccttcatctagttattgattggaaaatggcaatttgagagcctgaccacagagtaaattacacagtaacacagacaagctgaaggataatctgaaagacacacattgagtagatgacgctctttccttttacataaaactagtgtttcctctactaacaattccattgtgaccagatatttttaaaaatagttgaagacaaacatacgaaacacataaataatcatcacattattggagaagcataccttataattatcataaaatgactcccaggagaaataattcacatggcaagcatgtttaataaaaccaggacattttaactcttactaatagtggattttttttcacctttttcatttactttttttctgaactcagggcaatgaacttgtagacagctatatcaattgcagtgctatttctctgaggtattgaatctcagttattataattttgaaatccaattggcttggacttcattattttccaactaaaaagatgattgaaggatttatttgaaatgtgtaaagagtaatatagattttatgcttatgtttccttgaaaaaagtaggtaaaattcttctggaagtgttactcctaaaatacaaatgaacatgtcaagaattacataaattctttaaactatcacttatgaattattgcctctatgtagtgagtgacacctcagcagactattagatttagcttgcatggcaaagaactcatttagattcatgaaatggttctcactttcttggtaagatctggcttggacgtttttgttaattttttcttttttccttttttttttctttcaaatttggagatcttcatgagcagatgaatatttactatttcagatttgaaataaaccctgccaaactgctagaatatttattgagagaattttttaaaaattgcacaaattaacactggaaggtcaaactagaaaccaataacacacagaggaaggacagataatcagggcataagactggaaagatgacatttttaagaaaaccgtatatatagatttgacttaatcatgtgttcacttcatgtataaaaaaaagccttcaaatcagaaacaaatctaaaatataaaaaagtcatatataaaattagtcatataaccacataagatagaattattccaagtgacatttgagcaaagtgctgtgtctatacttccttagtcaatgttctgagaaaaaaaggaatatgaaacatgcaagacttttattagcaacaatttttatatttttgaaatttttatataaagattaagcaaataactaaagattttaatgttgttaatagaaacctagaggaaagttcataaatctttataaaagcaatttttaaaatttgtatttttaattgttccctctttcatatttcaggttagtctttaaaaagctgtttaataaagagaattccagatattatttccagtagtccatataactttaactattatcacttcaaaaataacaatagatgtatttcagtaattagattaattgcaattctctatatttttgcagttttgttttataacatgagtttggatgtgaacagaaaggcagagtctccaccaagattctatttctcttctaatggaacttctgacgtgattacaggaagcatacaggtgtccagcagagaagctaactgtagaacacatcaagcatttatgcgggtaatgtaagctattttttattaatgaatatgtgaacttcaacgttgttgatagagagcaacttattgtattggtatctttttataaaatgtagataaaagcaactgttacccctcctgaactatgacctgttgctccagttaaggtatgagacaatctcaacccaagtaattcagagtcctcttattgtatcatcctttttcacatttatcacatttgacctcatcatttgaaaacacttgccttttcctttctaaaatttcatttctcctatccctctttatctacttgcctagaaatatacatcttcagtagctcaactgagcctcaaatctattccgtggtacgttttttggggtgggtcaggggaggcagggaagttagtttgtttttcatacgactccagcgaagcagaatccaacatgactgcttttgggttatgtctttgtttccgtgccagctttgccagtgatgtcagtgggaactcacacagttgtagcatggtggggatcatctgtgggccaggacgatagctctcatgatggtttggggtatgtcctgccaaagcacagccgatttcatgtttcttttattaaaacactcattcccaaactgatcatcacattcctcacattggtcaaattttatcatatgaagctgtgccaggtctttttcattagggaacggtgtacttcatcagaaaatctattgtcacaaaaaaccaaactattcccaggagccctttctgggatttcaaatagtagcttcagcttagggcttaattttcccttagatacttattccaagcaaatgcaagcaagcatcaagactccaatacgctattaaatgctaagagcaataatccccatgctatttcctcatttcccttaacagtttttattgttgtactagaataaacctattagatggttcagagccaaaagtatagaatattaatgtaaccatcatttatatggtaggaattaaagaggttctttttttcatgcttcatagatgtcgttttacttaccttagtcaattgtagagacaaatattgaaatcattttaaaggcaataaaagggccaacattagatgttcacaaagaaatacattgtgttaaatttgtaaaagtgatctggtttatttagtcctgtgtttcttggcatcgttcctgtaaataagggatcatcaaaattgtacagctttttcggagtaaatttgtagcagaaatgattagcagaggtaagtgttgtgttgcatgtatatttgtagtgttaccatggctttgatggtagcaggattcagccattgcctttgtaccatattgcaaatattaaatgatagcacttactgctgggattgtacatggcagggactttttaagaaatgctcgtttccttcagtatctttttctcttcctaagttaccttgcttgtgtttggaacacttttaaaactcgtatatagaaaggcaaaaagttactccaaccctctcctttgtaacaaacccacacagaagataagaaccacagttgcagctaccctgaacttgagttctgtcaaggagctgtagctgttacttaagaaggtataggaatgtaatgattgtactttcccacctccaccagggtaatagcccaaggcattaggaaacattaacactcttaagtttctacagtctgaaagcataagacagcaacctgaattcttccatcttcctagaaaagaagacgcatagcggttgggatgcctgggttgctcctgtgttccataaaacgcatctggtaccatggtaccaggagtgggaatgttaacatatgtatctattttatctctgcattccttacaatgaaatgaagtggcacttgttaattatgtactggggcccacaacccccacttgtcagaggactccctgcctgtctgttggttggacatggtttacttgtgtagatgtccctactggccattcactccattaatttgataggtatttattgagtacccacattgtgtcaaattctgtatataccagtgaataagacataaaaacatcactaccctcatggagcttatagtctactggggtgagaaaaaaataatatgataaaaatatacattttatagtattttggaagatacaggtgataaaggaaaaaggagggcagagtacagaaatgtgcagtgctgcagaaggagtgaagcggagaattttaaataggatactcagaataggcttcattcagaaagtgacatctgagcaaagacgtgaagaaagtgaggaagttacccatgtagatatctagggcagttgttcttagttttagggtgtaatagcaacaacacctgtagggcttctgaaaacacaaattgctggcccccatctagaatccctgatttagtaggtctgcagtaagggctgagaatgtgcatttctgcaaagttcccagaggacccatactttgagaacagctggttcaggagaagagtattccagcgagaaaagctgagtcaaaaggcctcaagaatgtacctggttattttaaagaacaatggggaggcaaggatggctagaaatgtaagcaaagggaagaatggtggaagaagagattatatagaacctgtggctttttctctgagagaaagggggatctataacagcattttggcagagaagtcacatgatctaacatacgtatacaaaggaacactctagctgctgtgttgtgaatagagtaggagatcaagattcgacacatggagaagaggctactgtggcaacacaggtgagaggatggttccaacaaaagtggtggcagtggatatggtaagaagtagttgaagtcaatttgctgacatccaatttacaatttgctgagagactggatgtctggcatctgagaaagaggctctgtgatgactccagggtctttagcctgagccacttggaaatgtagttttcatcaattgagacaatattgcaggagaacaccttttgagtggaaaattctagagttcagtcgtgggcctgcttagctagagagacctattttaaatgtaaatatcaactaaggctttgtacatactcatcttgagttgaggacaggcagagaacaggaaaggtaacttaatatatctttggcatatggatggtatttaaagccgtattaagatggctgagatcagaagagggccaccgtctgtactctgggccgctacaacatgaagaggttggcaaaaagaggcccacagaggagataagaaggtccaatcagtaagaaaggaagaaaatcaacagtgtggcatcctggaagtgaagtgaaaaaaggggaaagcatgatgatgttttctatactgcagcttgatcaaataagatgagggctgaaaactcaacattggatttataatgcagaggctgttggtggtggtgacaacagcatttcagacattagtgggggaacggggggaaggaaaggaattggaagcgtcataggttattctttcaaaaagttttgccacaaaggtgaccaaagaaatggagctgggggtcagtagctgaactggagtgaagagttttttggggttttgtcttttaagacaggagaagtattagcatgtttttatgctgataggcatgctgcaatagagtgaaaaacttgggatatatgtgacaggggagaagagctggaactaaattcttgaaggcaaaaagggaaggattaaatgtacaagtgaaagggttggttctagataggcatgtgcatgattcatctgagtatgtgcttgcagatgctgggtagatgtggtgatggggactgtagaagttcttgtctgattgctttaataatctcagtgaactaagaagcaaactcatcaactggagtagggatggaggaagtggcatttggagtttgggaagatgtgaaatagtcttctaggagaatgggagagtaacaaaatacaggatggttgtcagatggtgtttgggaccacttgcacttattgtcataaactgagtgtagcctgtgtgcttgtgttttttttcccagtcacattcagtcttgcaggagtagatgaagagttacaattaaccagggttgtagtttggtcaagagagtgaagcaagcaaggagcatgcagcgggagggaatgtacaagggaatggttatatttttagttgtgaagttgaagcaggctaaggaggggagggggcacaccaagacggtgagggaccaggaaagaaatttgaattggagatcccaggacaatcagcaaattgttggaatcgactattaaacggatcgagctgcaaagatggcatagatattataaaatacaaaagcccttggagacctaaaagtgaggaaaagattgaattttgcttttatttttgctgacaatactttgattaccttatgcaaacaaaagcctttaaatttacttacaaaatccattgtgttctttgatggcggggaattcggctgtcaaaaagactccagatcttaagtttaaaaaattattctcttttaatcacaattctttagttttatgctagatcttcttggactagaggtgcatttttaatagtctaataaggatctatatcactggcatgtagatttttgttctggcatgttatttttctgaggctatgttttcatttatccggttagtaggggaatcagttttcttaagtctaatcataatcaaaagagtatcacacaggattactttgcttaactggtcaaaatttgttcctcataaaataagaaagaaggaagaaattgtctttttttttttttttttttttttgctttaaaaaatcctttgtattttagttgattataattatgccaagctttaattttggccaccagagccatacaattattagattttgctaatgagttagaatgatatttctgtaatataaaggaaatggttaatttcatttatatcttttagtgttttatctattgaaatcgtcttctattagacattgctttttcctctatgaaaagatgtttccccatttatgtaagtatatgaaaatctttgtttttgctttatttaaatatacaacttcatcaaattgtggagatttcttaggacaagggataattttgaagcagttttgatttagggaaacccaaactgtaacttgaagcagtttttagacatgttagagcagccatggtagtgaaacttcccttgaaattttgacagatgatatctcttggttgaacttgatggatccctgcatcaaaatatccactgatatttgagaggagaagaagggtgctacattgggataacaaaagaagcaatgagatttcctgtttgtctgcatttccaacaaagctagaaatactggcttagttgctgaacaaagaatgcctgtccccagagatattctgagagccagtgaattcagtggccttcgcatgttttattaaaatcatgacatcaatctacctgggtagattagttaattcttttctttgatttctcaaatttctgaattttagatttatgaaatgtgggtttattttgttatgtaaatctttaggtgacttctaaaactcatcttttttccatagtagtttcaggaaaacgtgtatttgattagtaaatacatttttttgtaattttttatttcatttgcttatattgttgagcaatacatgcacatagtttaatgaaatgtcatttactccaaagaagaaattttttaacaatagcacactatggactgattcatatatgcagacatttctgtggaaaatctaagtgtgagcaaactatatcactgtactaatattatggtattctaaaagtgaattgattattataacatgtgaattcttcattagattgcaagatctttgaggcaggatattatcttttgcatatttatattcttagagctagcacaatccattacataaggcagaaacaataaatatttaacaaatttatggatgctatgttcttggtgctatttaaaaaattctataagaaaaatctgagaatgttctattggtataatggttagggcatttgtcttaggggtgctgtcttttctcaggaaattgtaatgctagatgtctgactgttctgtaccacttgagccaagcttactagataaagggactgcaattatataagatggtgatggttcaggtaccaacctgtgtcttggtctcagcttttccttcccgagccctgcattagcaccagagaggtgtttccaaaatctagtcatattccttgcttaaaatcttcctatgtaagagcctccccagccccttttatgtgtagattaggtggccctacttcttctgtatttagacatgcctctgtcagaatattcatgccccacactgtctatgtggatttgtggaacatagattgtgacaggtactgactttcagttttgtaaccctagagctgccttggacacagtaggcccatgaatagatatcttcaaatatttattcctgacattattttttatttagtgatatcttataaatttttttgccccaccaagttaacaggaggacatgggcctgttgctcagctctcaagttggccagaaatacagatttccatgaaagtcaagcaagaagacagacattatgtaatttcaggcatgtgctaatttgcacgcttgtgggcaccctattttatgtaattgtccataaaaatgtggcatggaatcttcattatttttaatatagatagcacatgcccatccaaattttaaaatgtaaacaaatctacttttttactcccaaatatttaattaatcacttcaagacatttttgatattacagcttttgtccttaggtggagctgttaaagttaaataagtgtgaatatctgtcaaatacagtttttgcaagagtgcatgtacattttatatattgtaagaaaagcgtaattaatagctaattaggataatgaaagtaatatacaacagatgtagagaagaccttgtggaaaattagtatgaattagatagcatgtgatttggtcaaagtaagcttgttgagaagaattaaaacagtggacatgtggagggacaatcagtggctgtctagctcctcctacacctagggaatgatttaatcaagtttgctattacttgaatactcagggcttcaagctgttccgttttcacaagtcgataacttgggatttggccttgatttgcagcattcctatacggtcaaagtttcatgttgagatgatcacttaccaagtaatttaatataagaatctctgtataagaatggaaaagtaatcagggatcttgagtactctcaggctccaaatcttctaaataccgtttaataaatagagctctattccttgttctataactaattttctatttaatgttgatggttggcagtagtagttcagttttcttattccttccaaatgttcaccagaatctcctattctaggaaactgaaatttagtaatttcttagctggcttatatacctgaaatttgaagcaacatacaaatcttgtcaggtctgctttatggacattatgtcatttaatcatcacaacagtaaggtataagtataaccaccataattgttattattcctattttcagataacataactaagccatagacaaattaagaaacttgtccaatgttatgtagataataagtggtggaccccagatttgaacaccagttcttcaccttcagaccactattgcttttaaccactatatacataaattgataatgttgtaaatgaaaagagaaaaaagattacatcctttctgtttataattacataaagacatagaatagatatagtcttttatgtggtgtgtgtcatctttattaaattacttcgttgatttttatcaaaagttactatataaataatatagcaacacagcaataatatgtgtgatagggatttattccctatttcttaaggattattaattgatgagctttgaaaataatctattccttatctgctttggggcagtaatgtttaagccatattgcaatcctgaaatgtctatagataaatagaaacctggtccactgacattacttagtaggaggtctgtcaattgcagtgtcatgggtctaccttgaggaactcatctagcataatataaaccttaattatggttgaagctaatatgtctagcctgggtgaaaatactagactctttaatggtcaagagggaatttgttgaatctattttttaagatgagaagaacttatagattgaaacagccatatgaaaagtagagtcaaaaactgagccttaaattattagtatgaaagacatcacagtctgtgaccaattcatcaaattatctcctgcgtatagcaacaattctaataccaagaaaaacaaaaactgcatagattttctccacttgtttccgtgtaggtgggctatatgtggtgatgtgaactgctctctcatatcctgagatcatttctacttttaatttttgcagtgtctaaatcttattttatagatttctcatcgacatgtcttactttaggtttctcaattgaattctgttataaatcctcttaggttactttagtagtaaaatcttattagcttacccttttagataaggagaagtctgttttgttgagtaccctttgaaaatcacatttctgtctgcgtagtgcttgaacaggtgctttctaagagacactgtgttttatatgctacctgattttcatatacatttgccctgcagtaaccctgggggattgattccaggaccccacttggatacaaaaatccaaggatcctcaagtcccttatataaaattgtgccatatttgcacttaacctttgtaaatcctctcatatactttaaatcacctctaaattacttataacatgtaatacaatgtaaatgctatgtaaatacttgttatactgtattagttagggaataatgacaagaaaaaaaatctgtacatgttcagcactgatgcaaccatccattatttgccaactatttttgatccaaggtggctgaacccacagatgtggaaccgatgggtacagagggctgactctctttgttttcatagacttgagagtctaggacacttttactatctagtgaagtgcttacttaatcgaatgacaaatgctttcaaagatttttttttgtttatttcactgtgtgtaagcaaataattttgaaaataatgtggaatttttttaatgaggatagcaagtaatcaaatgaaaagaaaatcatttgtaagttgatagaaaatatttttaaaagaaaataatgtctgcatatagaaataaaggttaaaatgtaagaattgtcaatcagttgtaataaactcgagctgtttctttggagacagaagacaatgaattttaaaaatctgattattgaaaaaaaaaacttgtgaccacatatcaacaagaataggagtaagaaatgctgtgtagacataagatattaagtttaaatgaatgttatacagagcagaatggattgtgaatgggttacaatttctgtgtaactggtaaataaattcaagagttctttgtgaagtataaaagcacaataaacttagaaaaaacaaaaacggctgggcgcggtggttcaagcctgtaatcccagcactttgggaggccgagtggggtggatcacttgaggtcaggaatttgagaccagccctaccaacatggtgaaacctggtctttactaaaaatacaaaaattagctggatgtggttgtgcatgcctgtaattccagctactcgggagactgaagtgggaggatcacttgaacccaggagagagagattgtagtgagctgaaatcacgccactgccctcaagcctgggtgacagagcaagactgactcaaaaacgaaaaataaataaataaataaataaaaactttaacaactaataagaaaatcagtgaagcgaccagtgtatacaattagatgtacaaaacaaatacttttcctatataccattcagtatctgttagaaaagataatagaaaagttccataaagaaagaaaagggctgggtatggtggctcacacctgtaatcccagctctttgggaggccaaaactggaggagcccttgagcccaggagttcaagactagtctgggaaacattgaaagaccctgtctctataaatatgaaaaaatgggctgggcatggtggtacacatctgtagtcctagctactctgaaggctgaagcagaaggatcgcttgagcccaggagttcagaactgtggtgagctacacttcactccagtctgctgacagagggacacatcccatctcttaaaaaaaaaaaaaaaaaaaaaaaaaaaaaaaaaaaaaaaaaaaaaaactaaaaaggatgttttataataaatctggagtcaaatgaatcaaattcaatcaaaatgttgttgagagatataaagagaaaatgtatagggtaaagttaaatgggataaaaagcaccttgaaatgatttacaaaaagtttatcatgaaaaatgcttatgttataatatcaagttatcttttaaatgcgtagaaaatagcatggaagaaaatactcccaagtacagaactttataactattattggatagtattatttttcatttttattttttttgtgtttttcataagaaacgtaaatgattttgtgtgatatgaaaaaatatacatcttcatttttttaacggtttagataaaagataagtatttgctttgtcttccatgtatagtgtttggcccttttcaggaaaggagttttatttttttttaatctacgtgcttgtttttgttaattcatatggtggttattttccttagaaagatgtgcgggacatcctcaccccaattcagattgaagctgcttaccaccttggtcctcatgtcatcagtaaacgaagtacagaggaattcccaccacttcagccaattcttcagcagaagaaagaaaaagacataatgaaaaaaacagtaggaatattttcctttattcaaattattgtatggcatttaactaaatttttaaaatatggcataattctgaagaagtgaactatatgtcggaatttttaaaaatacgaatttttaaaaacaaaaatagatttattcatttctttaaattgtttatattccctttaggatattgaactgaatattttaaaatatgcatttaatcacaacagtagattctcatataaaatgctgatgttctatttttaatgccttgccagtgaagaaagatatattactagagagaaatcataatgggagaagattgtatttacaagttagttattcagtgtcatcatgtgtcctcagtttatcttcctcttctcattttctcccttgctttcatcctactcacagattcactcatgtcttcataatcaaattttcttgcttagttttacccatttacagacacatgtatttatgttatacaataatttctttttaaaaatttcattatatgcttttcagccacatcccaggatcaattgcatttgagatggatcctacattctaaccagtgaaccgttttctaaattatctcaaaatatttaagtttcactgacagtgtatttattctcaggagtcctgttgttcagacctttagtgtctatcaacacaaagacatagggtttgtccaaaaacaggatgtgtctgggcatagtaaaaagccttcattaaaatgttcactaatgacacacagaatgtaaaagatgaaagggacatttaaaggtctagttcacatcctgtccagacaaaattgccccatagtcactttaagataattgagcttacaatcaccactagtctagtctagaatacaggatcaaatgaagataactgaagagcagatttttctctacaccctcatatgaaaatatttacggaagtcaggggttgtttttactcttcttttgaacttcagttgtcaacatcaactatttgtaaatgtctttaatagtgaaaatctcaatattgcaagtacacaagccaaaaccttagcaattggtagttatattaatttatattttgattcttagaacaacctgctttaccaatagaaatggcagaaaaaaaaatcatatttatttagagagatatttacacctctcccccagctgtaggcacgctatttgacagtactctagtcacttcacacttaatgagcctggctccttctgactttctagatctctctttgttttctgcatctgtctctctctacctgttcccgttaccttcccttttatctctccccattttttccttttgcccagcttatgccttgggtcttcctaggtcctttgtagtatttattgatcttattaaaaagcttcgagttaattaaattctgaaacccttacttatttaattttgttcaatatttcctctaagtaaaatttctaagtatctccatgtagctaatttcaatgaatataaaatcactgtcccaacatctgtcacaattaagtgccatacttacacgtaataaatgggatcaaactgcagcaacagaaaattgagaggaacccaatgaaaaacatcttggcagctaataaactcctttaatgcctttcatttaactttttttccaaatcttatttcacttagaaaatttgggctttaaattttacctatttagaacttagctcttttagccctgcaagtaattgtcatttgaaccctactaattctcaaattgaactttttaaaagagataaaataaagagttgtcaatttgatgtatcaattttatttttctcatgcatcacaggtgtcgtcatctttaaaatatatataaatatacttgctgattaaatcaaaataaaaaacgttgtctttttatttccagataaactttgcaaggttttgtgcccatgaaaattgttctgctgatttacaggtttctgcaaagattgggtttttgaagtaagtatatgtggtatatagtctctgttattcatcgagagggtgtaatgagtgtgtgcatttgcattcccaaaagaaaaggaacaagagctaacccttgaaaattaacagcgtttaaagaattagcaacacaatgcttagttttttatcggccagaaaactggttatatcactcagactgatatataatccaagtatgacaaatttaaaaaggaataaactatcttgctcttctaaattcatagaaggggaaacagagtaaaaaggaaatagatatgtgttttagaatagcatctaacacatagcaaagactggataaatgttggttgctgtcgttattttataatggcaaataaaccactgcaattttgtatcatatctggatcccctatattcaaatcattttgcatatcaaaaagatgatgtgaaaacaaggaccagagagagagaatgaacacttcaggttgaggattgatcatagaaggcaagtgaaagaagggaagaagatgcatcagtgacagggcagggaaacggggaactatttttgggagattgggatataaatggagcagctttaatctggggcatgttgaatttggagggacttttctatgtggcattccacttgaaaatctagttcaggattatgtttggaagttcctccatatattagtgacttgtgagatggcccaagggatagaaatgtgaatttcacagaggaaaaaaattacagatcaaatcttggaggacagttttattcaaggagattaaaaagaatggggagtccctgtaggagactgagaaggcatcttcaaggaattaaagagaaccaagtccctggtgaaatagccaatggagaatataaaaagggagacttaatattgtaaaatttataaaaagatcagataacgtaatgtctaaaggtcatggaagttggcatttattaataaaatgtgacgggtggtgaaaaaatgggtatcactggcaaatgttaagatagaactctcagtgcagaagtagccaaatgacaggattagtgattaaagaaaggagagtatactgggaacaagtaggttcaagtgaggtatttttatgtatttatcaaatgttgttatttgttcaattattagaagatgtaaatgtgagtgccagaggcagggaaaagaataaattggagggagacatcttcaacttaacttccagcacatgcttttcagtccatacttgaattccactctgaagcatttcactgttcacttcctcttttgaatgtcttatcctttaattttcatgatactatcctactcccattgtcttagtagttatttagccctttcttttcagctgtttttgatggatctttgggtttggcaccctaaatggtcctccctcagccctttctctcactaccacattcaaccttggtagtcttgctcataactgcaatttcaactggcatgcctacactgctgatgcctaaaccagtgtctcttgagtttcacacccacctacatatcaaattccctattagacatgtccttttggttgccctatagtttcaatctgtccaggcactatccaggaatgacatggacattagaaagttaccaaagccagaaacctagggctcaatctgcattcttttctctttcttatttctgatatccagaattattaagtactacaatactaccacctagatgtctttcttatccatcatgctttgctatctctaccactattactccaaactaggcactcactatcctacctaggtcactaagttctcctgctttctaggatactggcctttgattttgcattattaacatacacatgtgataacagtgcttcagtggctcattttagacttaaaatctaaactcctgggtggtatttttcaccaattccccaggaagtttttcatcaatggctgaatacttatggtggtttggctgtgccatgctctcctgaacctctgtatcttctcacttcttctgcccatctacctgattaacttttatttatcttttaaatctcacctgtttcctaggggaagccccctggtcttcagctacttaaacttaagttctgttgtgcccttggtcctatggccctgtgctcacgattattgaaagtctgtctccacactgtcctggaagctgctgcaaagcagggtcctgtcatttttgtgttcccaatgcctaacagtgcctggagtgctgcaggtgactggagaggtttgctgagaatggaacaagatcccaaaagggatcagattcataggtagaggaattagccttagttaagaatctccttatcttgagctaacaagctatgaaaatatgtttgacattaagaaatttcagtgtggaaggaagggaaatacagagaactcatggatggctaggataaaaagcaagtttatatgtggtaattgaaggcattgaggatatgtttgggaactgaaaggaatggaaaaaattcaaaatagattaaggacagggagtcaactaggaataagtaaaaaggttgacaaatagggttgagtaccctactgtggtaggatcacatatggctgagaatcaattattttcacttaggcaatgtcagtttagccttgagagtccatagttcagggccaaaaagccttgacctatttgattgatgattggattttatacttaaattacccagattaactgtaatccagaaacagagactacggactactttgtatcccagcatgtagggcagtgcctttattgatttcttagttgaataacttataatcttcccatttctgtatttattttcttcctaaaccgcttaattatcttggtgtttttaccctcatgggcctttgataagaagacgagctgtgtgggggaaatcttttctaagctgcaagtaaaggagtgttttcattttcttaaatcagacacttgtgctatcacctttcattgcagtctctctggcctatgagaaaatgagacattgacatttataagaaaatattactaactatgcttctttcctgaataatgaaaatgcatcttaatgaattatttttctttgttacatgttttctcacaacaaaatatctgctttgcatttttaatttagagtgataatatttcttgcagcctatcaaacccattatgatttttaaatggcataatgattttaaatgaagtataaggctgtgtttcttaaggtgttgtctctgagccacctttatgagaatcacctggatgcttgaaagaaatgcagattcctaggccccagaccatccacgtcagaactaaaggggaccaggccacagatcttcacaattaatagagctccaagttcttatcacactgaaacctaagaaccaatgttatggtggtgaagagcttccagatttgaagtcaatctgacctggttcaaatcccatatctactgtattactagtgaccttttcagagaagacttttaacttctctagacctctttcctcattgctaagtgggtaaaaatagtaccgactcattaaggttgtcttaaagggtaataatgcaggccaagtgcctagtgcaaagcctgatttccatgaagactcaataaaagtcattactatttctatttttgaaatattatggttggaatttctatagataagcttttttagtttggccataaaatgaaacattgccaattctatacataattctgcatataagctttatatcctgaaaatatagctgctgtgactggctcagtagttgcagtcttacaaggtttgaagtggcatgaatgtccatataaagtagtaggcgccatattttttgtatcaactaagaaagttacaggctatgtatttctattagataaatgattatttgaggaactttcaaatgagagcccttctaattaatttcttagactcacagtgaaaggttctgacaaagtgctttgtgttttggtaaattatactctatcagacagaaatatgtctctcccacagaggtgtcctctctcctttctcagaaacagtaggcgaattgtgaccatatgttccatacattctcccagctataactctgctggcactaatggtatttatgtgccccagagctataaacctgttttaggtaaatgtatttactcagtaaagaacatgagtttctgaagactgcaataataaactgtcaacagtacgctaaacagtgcttatccactctctctcatctcttaagttgaggcttcctcctgtatgtctaaatgcttaggacaataaactgcatttataaaggacttgcagtgcaaaataaagatgaaccaacacgacttcaagattacaaatataatagaaggtattagccatcacatagggagtttaaaatcaaatccttgaaactcatgtaaatgataccgcaatacatttctgcacctcagttgtgcttccagaatgagtgatgtaagctttccctgaacaattgcttctgtatctgctaggaatacttttaattgcaagttttagagtaattaactaatagtgacttaaacaataaagatattatttcgcataataagcagtcttgaggaaggatgttcacaggtttggtgaacagctaccaaagactagacattgatggttatctttccagctctctcagcttttcctcatgatcacagtatagccaagcagcatcaagcttccaagcaagagaaaagggaaaagagcaaaagtgcttggtcatctttaagttgcccttccaggaagggcagtgcctcatttgatgtattctgattaaccagacgtgtgttacctgtttatccctgattctgttatggttgaaggagaatggagtcgtcatgatcacagtaaattgatcctgaatcaattattttgttattgtgatacattctctggagttgaacctagaggtctgtcttctgaaatattaagggaatgctgcctgtcacctgaataatctagggctctgttggcagctaagaagggaaatcactgttggctagacaatgagcaaggagcaacatctgccaccatgctgttaattcccgaaagagctagtgtgatgaaattgtccatttttaaaatggaaatagtacgaatatttggctcactgacagcataaagttttgggagaatgaattatatgctttataaactaagacatgctaattactggttcaatcttccaatttaagtttttctgctgttcttcccttaacctcagtgagtttgcctgtctttaataaaatcttcctaaatgtcaggtaacctgtctattccaacaggcaagaatttttcagtattgtctttttccctgaagtcagtagtaaaggaaaaaaagataaaaattgtacttaggcctgtagactacttgctgcaggttactcttttgtcaaataaatgagtcatgtatcttgacatagtgaagtatggtatcagtttacattatattttattctagcatcttgattaaaaacaagttagctatcaagccctaattcattttagggagacagggtgaattcattatactaagacttggggagcatttcttgcaaaataaagctatattattcactcagaaaaaaacatagagcctggtgctgcggtacatcatttttctatttttcctctgtcacttattaagttatgattctgtatgtacacagtaccttgatttgtttaagttacatggattctgtgctgctttgtaaatttcaagtctgcattatgcatgttttaatgaagaatgaactatgagtcccagggacagctctggttcagtcttgtcgcacttccagatgtgcacctggttctttattatgtctcacataggaagctggtaggtatctttctgagagcatcttgttaatgaatcaatttacagtaagatgtatatttaggggaaagaaagtagaattgtaattatggtaatggggcaaaacagcttaaaaatggaaaataaaccaggagtctagctgctgaaatgtgaacattgtgtttctctttaatttgctgttgtcttggaacctagctgtttatgcagtgctttcaccttctgttgcttgtctaagtgccaacttctttcccaaaggtgttcagtgactaatgctttggttacaagccagaatattttataaatgtctatttctgggcagctcttctacaagtcttaattaaactttaatgtcaagctgaagtttacttttagaacctcttaggttgctttagtcctaacatgattatagagtttggggtgggggggcaggatgtgggacagccatgttcccacacacttcctctctcccatttaacagtgccaccaagcagattcatactacttaaccccaaggtttacaacacaggcacgagtaaatcacagccattccctaaaatgttgtggtagcacactgtcactcagactcctccaccctgcagtgatacccagggtttggaattactgctttgttagttattacacattcttccagcatatccaactgctcaaatcagggatcccaatgtagagatgatgcatgtggaagtagcattctttcctttcttctccatgtctggaggagttatgaggactgggggatcattttctctaattctcatatggcttattggggtaaagaactgcatcctatgccatctgaaataatgaagcttctattatagtcagaatagtacaaaatagatttcaattaaatatggtaatatgttatatgtttattttgtaattaatttactcatcaattggattgctctgtagcaagagcctgcatttttttctgtgaagggacagatagtacatatttgagtttttgtgggacagagatctctgtcccaactactcaactctgtcactgcagtgcaaaagcatgtctgtgttctcatagagctttatttactaaagtaggcagacatgtgaatttggccttcagggagtagtttacagacctctactctatgatatgcttgctttttatatggaaattctaaagtatatatatttttattaattaaaaatagtgcctgatagtatattttgttttttaaacatagtttgtcaatataacttaggctggttgtgaaagtaagccaagattcaaagatttttaagaaattaatcctcagttaaaaaattataagaatctctcctagatagaaacaaagatagatataaatatgtatctagcctacagaattacgtgtgtatgttgaccaaaaaactataatagttccaaaaatggaaattgtcaaaagtcccatggataattgaaaggataaataagatgtggcatattcataaaatggactatatacaccaatgataagaaacattttacaactacatgtaccaatatgttaatctcacaaacaaaattttgaataaaagccatgcaaaagaacagatactcaaagattacattacatttatataaaatacaaaaccgaacaaaaccaagttatattattaaaaatcaaggagggtgaggacaataactggaagaaaaagccccagagggtttttcagtggtgctgctaatatttatttccttgagctaggtgcttaacgcatgtattcataaaaaatattgagctgtacaatcaggatatgtgcatttttctatgcatgtattatactctgataagtttaaaatagaaaaaaagaggaagaattttctagcattaagtgaagtgtccattttcttagaagttacattagagttataaaagtggggggaaatatccaaaattattttatttgcatattccgaggttcataagaataactctcaaaaaccgtaccacatttccagtttggattttgccatggtcatatgatgccattaattcataggtcatttcagtaggtttatcaccaccaaaacattaggttgtctggatgggggtacagatcgcaatgtggtaatgctagatatttactttcttagtgagacatttggaatttgtatagcatcacagaacagataaacacggtttaaggctataacgtgacagtatttatgctgatactttcatagacttctaacagtcaataacttatccaataatttccccaatttttttctgacatgttgcttggacaccaccgaacccttctttacatttaatacagacatgtacttacctgcttatattttttgaagactgtgttgttaggctctgagaaagcatgtgaatcacgatccagcatcttctccccaaggcaaattagtctagtagtgagaggaaccagagacttgacattgtgaaaatactgcatttttatagaggtgccccgtgtgctttgagagagggagaagctgccaccttacgctgttaggggagagcaatgcagggtgaaggtggttaacagtgagttttggctggaagaacaactgagaattattgacgtccaagataaatcttgaagaataagtacacgtcagatgcatgaagaggagatgttgacatcagagtgctagtttgaaggaagacgcaaaacatcctggtgtattagatgcctgtttatagaaaaatatgactgaaacaatgaattgtgcagtgaaagtgaagcaggggccaactctggagggcctctgtgtcatgctaaggagttggaactcttatcttgaaggcaatgtatggttctcaacagacaccattatctagtttgctttcttgaaaatgaccctctggcagtgtgaagactggattaaaggagaggagaaggacttgagacagataaatcaattagaaactcaatccaagtgaatttaggttcaatcatgacattttcagacttaaaatttcttgaggcagccaatttaaccatagtagtagagaaagaacaagtatcatgtctttactacctcaggcaaaaagtggtttcccagagccctctattcacaacacttgcccgtttgtgtatcttttttacatttatttacacatctgtcctttattatatcagctctaggaaggagtgaagtgtgttggtatttgggaacgatgaacttagtgcctgatttattgtacagaaactttcaattcattcatgaatcttcaagtaataattaaaagcagaaattttgaaacatgttcttaaactctgtccttacatactctgtctttaatcaattcacaaaaacaactaaattcctactgtatctttagtatttccataaatagtttattcaaacactaatcttctctacaaatgttgatttgaatgacacaatttaaacattattgaagaaactttctaaattctgcagtgctaggtggatatttatcttttttatctatcctcacctgatattttgctttgggtaaatgcttctctacttttaactaatttttctgaatgttaaattactagatatctaagaacttctgttcttaggggaaagtttataaaatgacatactgtattggaaagttttccttagcacatagctgttctaaccctctcactgggaaaaagaatgaactgtagaaatttgcaattgtttcaagacttcagaacattctagctgtggcctttgaataagttccttatgtctcaaatacttcatatttaaaatattttaaggactttatacagtatacataacccagatcttcagtgaatggtaggtagtatggaataggatgactgaggcttaaaaatgctaaattacttccccaccagaaagaactgctggcagtgccaggacttgaactcgtgcatatatatggatttacagcaatctatactttgcaatacacaggaggatgaagcatttctccagttctgtttttaaatttggcctagttactggtggaggtgaccagctgcttcacaatttaattctagtccaacagattttagtgccacgcacttgtgggtggacatagtagttcttcctcatgccttgctcacttctaccaagtattccttctagtcttcttttctaccagttccttccattttcctccctcggttcaggcagcagctagttcaatgtctattttaggcttgcaagaaagaaaagaaaaactgctttgcctttagaaaatgttacgaaataatcaggaaaatgttaaaattatagaagcaagaggttatgcagaaaagaaatgagaaaagatacgagtttaattatagctcattaaagtttccttgggtaataggtgactgaatttataaaaaacatgacaaagagttcttcttaatatggttttacatctgcttgcatagggtcaatcccatgattgtttttccaagaaaattagtattttatgttcagcataaattatatacatatatccttgtatgcatataagagagagggatttttatttcctaaacaagaactaaataatattacttaatttttaggccccatgaaaataaaacatatcttgctgttgggagtatgaagacattgatgttgaatgtgtccttgtttaatgctggagatgatgcatatgaaacgactctacatgtcaaactacccgtgggtctttatttcattaagattttagagctggtaagtactgtaaaccacaatagcatgatactacttggtattttacttaattttttaaaagtaaatggtagaattcacttcactgatttggggtataagtcttaatatttaggctcctagttattttcagacttactctgtgcccctgctcagaatggcaagttcttgggtcttaagtccaaaggcatcagcctcctgttttccagtactaggagtaggaggaagctgtttgggtgcctttacattctcactgccgtagccagcacacgtctgatgttggaacagtatccccttgattgtggtccttgcacaagtaacattgatcctccatcctggactccagagttatagttctttattttaaggtgacactcagacaattaatagaattttctctattctttatcaaattcgctcatcttttgtccagggaccacaccttcataccaatagtgacctttgacctctcacgaatatctgttaccttctttctctagtgtttgcgctacttgcaaaagtaacagtggaaactcagatattcacggacagtatttttaaagcattttatacttattgcaattataacctcattacacctattgagattatagctattttgattaaataccttaaacacattctcacataccagacagaagaggaccattaaaatatgaaccttatggattccttgcctcatgatttttcaaggaattcagaccctggattaaatagggaatatatcaagataaatgaaagctacaagtccagaattttttttttgtggagaatctgtttgaatactagagtacacacatatatgtatatacacacacacatacacatacacatatatatacacacacatatatacacacatatatacatacatatatatacacatacatatatacacacatgcacacatatttatatcatatgtctatttatctcaaacatataaatagaaaatagttttcctagaaaagcaaatacttctgggatgatattcttttcaataaccatccttaaacatatgttacaaactttttatttccttcctgtccaaaacagttgtttcatttttcccattaggaagagaagcaaataaactgtgaagtcacagataactctggcgtggtacaacttgactgcagtattggctatatatatgtagatcatctctcaagggtaagtgtttcatatttatggcttttgttcactatcatgaatatttttttctattcttccctatctttaggttgcatagaaaatattataaatattttagcttggggtaggtagctgagtgatgaaataaaactggttcttgaaatctttgagctttgtgtttatattcttggcaatgtttgctgttttaaagggggtggcatgtttacatcgaaatgggcatgtgcatgtgtcaatcagaattctgctccccctacacacccttcccgaaaacccccacccccaccgcaggtggtcctgtctgccaggcatgttacctctgctatacaaaaaggtgtttttggcaagagtctccactcaagttgtgaaagcatttctaattttgtctagacttgcctgcgttcacattcagagacgtctttgtctctgaatgttacgtgtggatatgtgtgtactttaaaatagccacaaacccaacaacttccctgaatcttattgccaagggaggagtagctgatgcctttacaatggttcaattctacattccatagaacataaacttttaagaaaaaaattcagattataaaaaatgtacttaagattttttttaatgggctttcctggtctgtgttttacagatagatattagctttctcctggatgtgagctcactcagcagagcggaagaggacctcagtatcacagtgcatgctacctggtataatttattgttaataaaatgaactagaaatatacccccatattctgaggggggggaattaggagaaccgtaaaactgtgttccattaattgtaagagaaaacttctcttacgtggtatgcttttaaaagaaccaaacaacatagttaaatgggagttactggcaatgttttagctcttgagctatgttaagtatttattagattagattagagtagacatgatgtgtcacgaatcaaactgtgtatttgaaatcaaaaacagaataaacagtttacaagtaataactctacaagatttaaaagtgagggtaatgacttccatagatatgtctcctagagtaaataggaaatcgttaatgactatttcatgagtgaagtttcaatatattttttaaaattcagaataaacgtcaagatggacttcattttacctaacacaacaacttggtcaccctctcatcctaaaaatctctataagtaaaactgattataaagatacgttaaggcagtacatgacttgctatacagtttaaaaaattatatgattgatgtgctttctttgatcattaagtcttgcaaaaatacttcttcaacctaaagagagtaatagatttgtttctaaactacctatttattctggtttttgtaccacctagaacataaatgttaaaaaatcttttaatctaataaatgtaactatttgttaaaaaaaaaaaaattcctctatctagcattagaagcaaatgataacagtttaaataatgagagaaattttctgccaaccttgtcctaaaaaatttttactaccatatctacttttatccccactcctcaaaaagcatttaagcaaacctctgagtatattaggtatacagggtatgtaattagttctctctgaagatttttctgcttggtgaattctaacagggtgttttctgtttgtatagtgaaaatgaagaggaaatggacaatctaaagcacagcagagtgactgtagcaatacctttaaaatatgaggttaagctgactgttcatgggtaagtagacataaaggcttcctttcaaatttagagctgatttacagtttccttgcttcttacattaatgaaaaaactgtattctagaatttttttaaaataaaaaggatactctatcttttaataattacctcattgttatagtcccttacttagctgtccattataataatccactctataatcatcactgatactacatcaaactagtacccagctggaggagatttcttactgtgagagccccaacaagctagcaaggccacactgagcctttcagctgtgcactgtgtaatcctaatctggaattaggcagtatgcagtctgcataatcatacttaatgcccatgtcaagtgtgaagttcagagaagtgtgaacttagtcaaatcactattctgcagacccgtatctacacatgtggcaaatggattgagatgtgagagatctggctgcaaggtgtcccttgtccactttttatacctatgggatcaccagattcctcaaccacagtatcagaattcattgcttcctcatgattttaatgattgagtggatccccctctcattcaagaaggagatgtgtggagaaagaataaaagggggcttacggaaaccttcagtaattccactagaacttattcatggtgttagtccttaaaactacatttttatgcagtagaagatatgagttacagaacaaatagacagatgagcctttcctgttgctctaaatctaaaccatcccgcagtcatcttatccacttagaaaggtgatatacagtcatgtaaatgtaaaatgtcagtgtctgatggaattgagtatcaagtttgagataaagcaaacgggtgaaaaaaggccattcacaggagagtaagacctagagaatcacgagtaagcaaataaaaagttcattgcgtgtttcctggttttcagtgtttatcttgccactcctcctggcttatgatgagtgtcttcctatttttctgttattcacatctcattattttgaacaaggtatgttcacctgttaaactgcctaaatctatgctggttactttcctgtttcatgagtttctaatttggtattacctaatattagctgccaaatgtgagcaccttatggagtagagatctccctagaaacatatttcttagagtttattgcagataaaaatgtcaaatcacaggcctttaaaagagagtgacagccttgtattgtaacaaaaaatttatcatacttaataatgttagacttgttccccagatttggggttcattataagtagccagcagggatcatgagatgggaagaaataattcaaaaatgggtaacataaattgaggtatgagaaccttaggaaaaaactaagaagttatcccacattacatttgccaaatttacatgaatcacttaagggctgaaagagaaggaatggtcagtagaatgaaaaaggtgtagtgtcatggaaagcaagaaaagaatatcggtgagggacagatattgtcacccaggtcagagcacatggccttttttttttttttttttttttttttttttttaagagtctcgctctattgcccatgctggagggcagcggtgtgacctcagctcactgcaacctctgcttctcgggttcaagcgattctcctgcctcagcctcccgagtagctggtactacaggcacacgccaccacgcccagctaatttttttttgtatttttagtagagattgggtttcaccacgttggccaggctgccctcaaactcctgacctcaggtgatgcgccctcctcggcctcccaaattgctgggattacaggcatgagccactgctcccggccctaatttaatttctttttaaataaaatgtatatctgaccacaaaaccaaaagtattccatggtgacttgtaaatatgctataatccagactatagaaaaagactctttataatacgtcatcgaataagacagttaattaaatttgaatttgtttttaccaggtttgtaaacccaacttcatttgtgtatggatcaaatgatgaaaatgagcctgaaacgtgcatggtggagaaaatgaacttaactttccatgtaagaaaagttaattgtgttaatatttgtaacaacctaaaaggatgtcactgatgcattgctccaagggacattgtacacctatgacgtccttcagcctctgcagaccacagctggctggaatggggcagcagcagtggtactcctgcctctcagacccctgctgctttccttccacttgtcttgtccctgatattcaagcccatatttccttacaggacaggtgagaaagagcatgcaggaacagagataatgcctgaaacaggcaaagttatgggatgagctgaatttgctagagacacatacagaaaacaggacaggccaagggaaggcatctttgggtgcaaatcagtgaaatttcttcttggttcaagcgtataaaaacctcagcactacaaagtaaaggaggctcttacctcccatctccatttaagatcacgttggttagcctttcaagccataagtttatgacctgtgacaagtgctttatcaattattaaataaccttttaaagtaaagagtactatcgtaattcagaagtattaggagactgatgtaaacctaagtttttaaaaacattttttgctgtcaaactgatatttttattgtatcaaagatgttggtgattagtaaatataatttccaaaaaaaaattctttgaaccaaaagcactgtccaagcattcttatagaataatctatatttgtgagtaaatcttggattaggtataatattttgtaagacaatgcttttcaaaatgtgtattagaatcacttggggtacctatccagaatgcaaattccttggctttgccccaaatctgtggaaaaagaatgaccttgaaatcctgttgattctatgtatactccaatttttgttacactagttgatactaatgttgaaaaattttagttctccactttgaagtcccaagaaatgctctttaaaacccaatgggagtttgtgctttgataaactaaaacagaaaccaaaccggactctagaatgtttatttattaatttacaattaatagttaaaaagtattttctactaaatgtataatgtagtgactggcactggagaaacttgcagtatataggatttctgtgtctagtcaactatgtagatattcttagaggtttaattgttgttaggcaaacagatgtttgtgatttagatatttaatggcatccagaagaccttgtctgtagtattgggcactgctaggtatctgttgctgacatttcctgctgcaactgccctgtgcttttatggattagcaattaccacctgttcccaatttggagacactttcttcatctatgtcacatgcattcataaaatgactgcaggtctgcaagtcatcttttttaatcgaggtattgggatgcatctctttctgacttactgtattggttttacagttaagctgccagttcagtaggctatattttggccccatttcccttttcaggaatcttcaatccccaaatttatcatcctcttatgcattttttgacaagtattgttaaaaaccttttgtgctttctcaaaacccaacaacatacttttcttctgaatgcatcagcatatagatcctgaataaaaatacatttttattcactttctcctgctgattatccccttagcacagagtgcttttgcgataacatcataggatttagaattacaatacctgagttttggtcataactcttgtcattttttaacttttatcttgggaacactaagaacacctgttagcatttcccttagctggtgaataccaaaatgttggcctacctcatgagaaccaaatgatatttttaaaatatgtaatgtaacatacaaatatatattgtttatgtctcctaagactgtttctccaataaaccatcagcctgtactttttgttttcaacaataccaaaactagaaggttagaaacaatgactcatttgaaattattagcaaactttaaaaattctgactatggatatcctgtctccctttgttatgcagctgccaaccataaacctccaccactataaatgttgcatggaatatacgggatcacctaaaagctcactgatatctgtacttacatttatagaaaacatttaatttgttaaaaaatttttccttcttatcaggttatcaacactggcaatagtatggctcccaatgttagtgtggaaataatggtaccaaattcttttagcccccaaactgataagctgttcaacattttggatgtccaggtaaaaatgtatttcttccatctaacctttattgtgtgtcttaaatactaaaataaatgcaaaccaatcctttattcgagtaatgatgtgaaaatggataattgttaacttacttcagtcacactctaagctatgaattcaccatgcatttattgagttatgctttgaatattatacatttctttagaactatagagttacagtgcaacttttacatttgatttaaataacagaactatattctaccatctccattgaataattattttaagatacattgttgtgtgatatggacctatagaggtaacttctattagtagaattcttcataaaacttgaaactactgtgcaaatatgaggattatagcctttttttaaggaagcactattcaaaccacttcttaagaaccgatattcttgcactgatggttccatcagaattgttttgagcttaacaggttgaagacaaaagtcaacaaatctactaaataggtttgaggtctgatttttaaaaatcctatttaaattccagatttagagagaagaatatggtgttatattgtttcgcctataaaggcaaagaaatatttaatgtcatgctaatgttccaatatcatttttcacatttctgcaaggttaaggccattttatcagcttagaacctatatccaacctgctgtaatttggctgggtttttgtgtttctgccacagacatttaatcactcccaaatctgtcaaaaaaatttctacaatttcagaggtacttgatatattttagcaaagataagctactggaacttttaaaaccaggttgctttttgctgttttttccagtgttgaaagataagatttctcttgctttctgtcttcatagactactactggagaatgccactttgaaaattatcaaagagtgtgtgcattagagcagcaaaagagtgcaatgcagaccttgaaaggcatagtccggttcttgtccaagactgataagaggctattggtaagtttcagtttttcaggttgtagttcctgctttccaacagagaagtgagacacttaaaatcaagtcaatgggtttgagctgtcacttcaataataagagagaagacaagtttaggtagtattcttgcgtggaaggaacaagcatggttggaaagttgagatagacccagggtctatcccatatataaacatgttggccgggtgtgatggctcacgcctgtaatcccagcactttgggaggccgaggtgggcagatcacaaggtcaggagttcgagaccagcctggccaacagagtgaaaccccgtctctactaaaaatacaaaaattagacaggtgcctgtaatcccagctactcaggaggctgaggcaggagaattgcttgaatccgggaggtggaggttgcagtgagccgagatcacgccactgcactccagcctgggtgacagagcaagactccatctcaaaaagaaaaaaaaaaatgttatacagtttcatttttttatctaacaaatgcccactatttgccaacaagacattgttccagacccttcaggacaccaagaggttgtcttcacctgggcagcctttgtggcatgggttaattttcattcttttcagcttagtgaatgtatgaaaagaaactggagaagcattcatcaccttgaaatgatccacgttttacaacaaacaaagaatacgtaacttagcaagactctgattaggaacattttttaaaaattctgtcccagaagattatgttaatacaaataataagaacatatgctgcagtttaaatactttattataatttagaaaacaaacattcatgtcttgtttaaaggtaatttaaatgccactaaggaaagcagatgtttaatttcatatttatggaaagatgattttcaaaatgccattagagtatatattaaattctatataaaatattttaaaaatattttccaatgttgaaaattttaatgtagcacttttatattcccttcaagtactgcataaaagctgatccacattgtttaaatttcttgtgtaattttgggaaaatggaaagtggaaaagaagccagtgttcatatccaactggaaggccggccatccattttagaaatggtaagtaagtctaaaacattgacaacttggtggctaagtttacataaaatctataaattcagtcatataggcaggatagtatgaaggcattcaacaaattaaattttaaataaaattttggagtaaaactctaaaactgtatttctatcacttcaactatctacattgactaagtgaaatggctttagaaattaacaggtaataaaggctaaaataggccaggcacagtggctcatgcctgtaatcccagcactttgggaggccaaggcaggaggatcacttgaggccaggagttcgagaccagttgtattcctaggtagatgtctggtgttatttctgaggtctctgttctgttccattggcctctgtatctgttttggttagccttgtagtacagtttgaagtcgggtagcctgatgcctccagctttctttttgcttaggattgtcttgactatatgggctcttttatggttccataggaaatttaaagtagttttttctaattctgtgaagaaagtcaatggtagcttgatgggagtagccttgaatctgtaaattactttgggcagtatggccattttcacgatattgattcttcctatccatgagcatagaatatttttttccatttgtttgtgtcctcttatttccttgagcagtggtttgtagtgctccttgaagaggtccttcacatcccttgtaagttggattcctaggtattttattctgtttgtagtgaatgggaattcactcatgatttggctctctgcttgtctattattgtatacgaatgcttgtgacttttgcacattgatttttgtatcctgagactttgctgaagttgcttataagcttaaggagttttggggctgagaccatggagttttctaaatatacaatcatgtcacctgcaaacaatttgacttcctctcttcctatttgaataccctttgttgctttctcttgcctgattgccctggccagaacttccaatactgtgttgaataggagtggtgagagagggcatccttgtcttgtgacggttttcaaagggaatgcttccagtttttgcccattcagtatgatattggctatgggtttgtcataaatagctcttattgagatatgttccatcaacacctagtttattgagagtttttagcatgaagggatgatgaattttattgaagcccttttattttaaaaatatagctaagaagaaaaactaaggctttcataaaatcaaataaaatcaaaattcaacaacaaaaacatatttaaattttaaaaataatgaaatatatcttggactttttcctacaaaggagcatgtatatttatgaggagtagtttgagatgaaactagttttatgtggagttctgtttttcagctttctcatctgttcaatatggataaatgagatgatttttataagcacttagttcatggaacataggaagccaaaggaaatggcagctgttatgttcaagtggaattcttgaaattgccaaacagtgcttaagaggaagatacactagacaggcaaagaagaaatatccattttagtttcaactgtgctaaaagcaaagtcattgaaccatcagcaagtcatttaaccttttgcagtttgccttatctttttctctaggtctaggttttccatcagtggccataattggcattttgaaccagataattttgttttggggggttgtcctgtgcattgtaggatatttagtatcatcctcagcctctatacgctcactgctggtagtaccacctcccacgttctgacacttgacagtttctttagacattcctgactgtcccttaggtgataaaaccctccctgctgagaaccactgctctagatctataggaagcttgtcatttttggctctaaaatgttaaataacatttacacaatagttttaaaggtcatatccctctttaagaatctgactaaatatattctcatctccctcagcaaatgcacacatgaaaacatctcctaaagattcttagaatttataaagcccttctacagatccccaagcttcatggattctgcgttataaaatccttgctttagatagtaaagttctgagttcatccactgattttatatttgaattctataatgtatttaatttagaaagaaataaaaagcccaaataagatagattcaatatgcggtggtggggtggagaacaatggtaaatggtaacttacctctttctgtagtctaatatttaaatcaagttatttgcaaggtagctgtttaatcaaaggatttcaataaaatgatactatataaattaataccaagaccacactttgggaaccaaaggaggcactgcagttaattttaagtggtgaaataaattggggaaggtaaattgtgaaaacctctagctagaaggtaaagatcctgataaattatgtctttatgaaataaaccaggctatggtgatccttcttttattaaacaggatgagacttcagcactcaagtttgaaataagagcaacaggttttccagagccaaatccaagagtaattgaactaaacaaggatgagaatgttgcgcatgtaagattaccctcttaactgctacattaaaattataggaaaacacatttcaagggtgtctataattacttccttctgagtaactgagttgggaaagtagagtgtgaccagctcatggagggcccccaatacttggttgagagttggcattttccacaactggcaggagggaacaatacagcagttctttcagagaggtgaattgggcagcagtgagaattgaatagattggaggaaaagtcatggatacagcccattacactcactgtttctgctaaacagttaccagtgtttcctgcaattatattagaaccagcaagaaaatgggctgggcagttctaaatactactggggattatggcagggctttaaaggaaatataagcaacgttttaaactgatttttttttttggtttttgagttttatttttcttaactcacgtaggttctactggaaggactacatcatcaaagacccaaacgttatttcaccatagtgattatttcaagtagcttgctacttggacttattgtacttctgttgatctcatatgttatgtggaaggtaagcatttaacaattaccaacattagtctactaaaaatgacattttctcaaagccaatttgacttccaagttattagatttaaatatttcactatttgaatgttaactttttatgttgctcagtactgatctcacatttctcttcaacaccaaagtctttctagaaaaatatgcattcttaatttgttttgaaagacagacgttccttttaaaaaaccatttggaaaagattgcaaattacacggaatgtcaattttggagagaatgtggtgtgtatcactgagaatttctgcattaaaaacaaaaatgtttctttaacccagtatgtccccactaatttttactgattacctctgttaaacctttatgactgatgttacaagggatgcagtttaagaaatattggtgttaactgcttagatattagaaatgagtatagtagataagagagtgttcataactatacactagtgattatgttatgctattttcaggctggcttctttaaaagacaatacaaatctatcctacaagaagaaaacagaagagacagttggagttatatcaacagtaaaagcaatgatgattaaggacttctttcaaattgagagaatggaaaacagactcaggttgtagtaaagaaatttaaaagacactgtttacaagaaaaaatgaattttgtttggacttcttttactcatgatcttgtgacatattatgtcttcatgcaaggggaaaatctcagcaatgattactctttgagatagaagaactgcaaaggtaataatacagccaaagataatctctcagcttttaaatgggtagagaaacactaaagcattcaatttattcaagaaaagtaagcccttgaagatatcttgaaatgaaagtataactgagttaaattatactggagaagtcttagacttgaaatactacttaccatatgtgcttgcctcagtaaaatgaaccccactgggtgggcagaggttcatttcaaatacatctttgatacttgttcaaaatatgttctttaaaaatataattttttagagagctgttcccaaattttctaacgagtggaccattatcactttaaagccctttatttataatacatttcctacgggctgtgttccaacaaccattttttttcagcagactatgaatattatagtattataggccaaactggcaaacttcagactgaacatgtacactggtttgagcttagtgaaattacttctggataattatttttttataattatggatttcaccatctttctttctgtatatatacatgtgtttttatgtaggtatatatttaccattcttcctatctattcttcctataacacacctttatcaagcatacccaggagtaatcttcaaatcttttgttatattctgaaacaaaagattgtgagtgttgcactttacctgatacacgctgatttagaaaatacagaaaccatacctcactaataactttaaaatcaaagctgtgcaaagactagggggcctatacttcatatgtattatgtactatgtaaaatattgactatcacacaactatttccttggatgtaattctttgttaccctttacaagtataagtgttaccttacatggaaacgaagaaacaaaattcataaatttaaattcataaatttagctgaaagatactgattcaatttgtatacagtgaatataaatgagacgacagcaaaattttcatgaaatgtaaaatatttttatagtttgttcatactatatgaggttctattttaaatgactttctggattttaaaaaatttctttaaatacaatcatttttgtaatatttattttatgcttatgatctagataattgcagaatatcattttatctgactctgccttcataagagagctgtggccgaattttgaacatctgttatagggagtgatcaaattagaaggcaatgtggaaaaacaattctgggaaagatttctttatatgaagtccctgccactagccagccatcctaattgatgaaagttatctgttcacaggcctgcagtgatggtgaggaatgttctgagatttgcgaaggcatttgagtagtgaaatgtaagcacaaaacctcctgaacccagagtgtgtatacacaggaataaactttatgacatttatgtatttttaaaaaactttgtatcgttataaaaaggctagtcattctttcaggagaacatctaggatcatagatgaaaaatcaagccccgatttagaactgtcttctccaggatggtctctaaggaaatttacatttggttctttcctactcagaactactcagaaacaactatatatttcaggttatctgagcacagtgaaagcagagtactatggttgtccaacacaggcctctcagatacaaggggaacacaattacatattgggctagattttgcccagttcaaaatagtatttgttatcaacttactttgttacttgtatcatgaattttaaaaccctaccactttaagaagacagggatgggttattcttttttggcaggtaggctatataactatgtgattttgaaatttaactgctctggattagggagcagtgaatcaaggcagacttatgaaatctgtattatatttgtaacagaatataggaaatttaacataattgatgagctcaaatcctgaaaaatgaaagaatccaaattatttcagaattatctaggttaaatattgatgtattatgatggttgcaaagtttttttgtgtgtccaataaacacattgtaaaaaaaagaatttgaattgatatctaaaaacagaatttgaattgatatttcatcttgacttttaaagccctagaggctaattgttagtaacatcaatttctattaggatatccgtttggccacacagcaggaggttagagcaatggagcattactgagttcctccccctgtcagatcagcagcagcattagattctcatagaagtgcgaaccatatggtgaactggtatgtgagggatctagagtgccatgttcctcaagagaatctaatgcctgatgatctgaggtggaacagttcatcctgaaaccattcccccatccacggaaaaattgtcttccatgaaactggtcccaaaaagggtggggaccacaggtttaaagcatggccacatttctttatattaaaattctagtttgtacatttcttttagaaacaattacatgttactttggaatcatttcttccatgcttcctccataaagactgataagtcttggatgcaatctgtaaagaaaatacattatttcatcaacttattttgttgtttttcacatacacctaataagtatggtacacaatgccaatgccaaatacaaattgataacaaacacagcattcccaacagagctgtaatctagaaaactgagaaggtctgattgataaatcatcaacaacaataattgctctaaaacctccttaactgacttccttgattgtccaatgctctccattacctctgtaaaacagtcagttatgcctctagaacacccatgtctagtgggcacccctgcatgcttcttctaaccactgagtgtcacaatgcctaccaagaatgcgtttgcaggttcctaaacctgtttataccagttgctatgtaaaattgttcccaagggaagttgaatgctctgtaaaggcctaataaaagcaaattactgaacaaaacatgttacagtaattatgagtgagaggaaactaagatggaaggataaaaatctaacactttactattcagatggctccactaaaagatttaagatcttgatccatttttaaaaatccaaaatggaagttgtagacattatctgtagtttatgcacaacaataaattagaaagccaatgtagacacgcataaccaaagaaaatgccttgggtctacataacagttgaataaatgtaaagttgctttt

SEQ ID NO: 2 - protein, 1032 aa, Homo sapiens [sequence omitted]

SEQ ID NO: 3 - protein, 195 aa, Homo sapiens
MAWEARREPGPRRAAVRETVMLLLCLGVPTGRPYNVDTESALLYQGPHNTLFGYSVVLHSHGANRWLLVGAPTANWLANASVINPGAIYRCRIGKNPGQTCEQLQLGSPNGEPCGKTCLEERDNQWLGVTLSRQPGENGSIVTCGHRWKNIFYIKNENKLPTGGCYGVPPDLRTELSKRIAPCYQGSISKYRART

SEQ ID NO: 4 - DNA, 16058 bp, Homo sapiens
tccactcccttggctcagccccacccctatctgcaccaccttctagatcagtcacccccaccaaccccaaatcttccccaccctggggagtgtcacttcctcctctgccgtctcccagatcagtacacaaaggctgctgctgccgccagaggaaggactgctctgcacgcacctgtgagtaccagggcaccagcccttctgcctcctctccttacaagctctggcccacctggtcccaacctcttgggaagagggctctgacctggataccgagggtcctgtccagacttgagaaaggctctgaactcctttctgcattcctaagatcccctgacctggttccctcctccagtttcacagtacccccacctccacctctccaaaagcccaatctctcctgcatcagtttcccatgagtgggtctggaggcagggatgtatatgagtgctgtcatctgagtgcctgccatgtgccaaggactataccgaagtagggcaggtataccgattatactgagggagggttcttccaacatcacccccagctgctatccctaatgctctgccttcagctcagacttcttcctaatcggcaccttggtcacagactccactgtcctgggacactgaggcccagcagctcagttccgtcccctccttgtgactcccagtgggcagcagagggttctgcttgatttggaaggtagggatgggtttggaagaggctctgtttctcagctgctgaggggggtcacagggtgtgtactggagtcagaatggagtccagaaaaatggaaataaccacttggtcccagagagagaagggaagactgcagaatacccctaaggaaagagaaatgggattaggcagagcccaccagggctagtggggagagaacacaacttttgatggagatagtgcctgggttggagggaggaaaggagaaagcacagcagaggcctggccctgccctgtcctgccctgcccctagttcactgggtgaccctggcctcatggaatttaggtttccatttgtaaaaagagtgggatggagtagtagattcagccattctatagttaggaacagaggggaaaaggaagtaaaatagccctatgaagaagtggagcagagaaaagcagcagagagatgcaatagaagttttgaaatgaaaggagccagagagagacagagaagcagacagagaaataggtggcagaacatgtattctcaaggaggggagaagagaggataattggaaagaaaggtaggaaagggctgtccaggaagtcccagggccccctttaaggggagatgagtgggcctaaggtctctgaaaaacctatgcggcagccggggctccacttgtgagagatgagtcaaaacccggctttgggttgcagcatctccagcctccctgaccacaggcatgcagagcaagcagggctgaggttacgctgaggctgagctgggactggcctctccccgaggggtggaggctccgttatatatggcgcctgaggtcacaggtggtggggagcaggaccaggacattcaaggactcaacaccccacccctttcccacactctgtgcagagtatctggacctgactctgggcccaggctgaagaagatgcaaatggtttatgacaaggagttagttggcctttctcatacattgctgatgggaatgtggaccagtacaagtcctgaagagtttatgttggcaatacccacctatatatatatatttcttagcatgagaatgtagatgaaatacacaccagtattacaaatgatacctatctagttatcctgcacgtcctctgctagggatttatcctacagattcagttgcagacgtgtgaaatgataaatacatgacatttggctgggggtgatggctcacgcctgtaatcacggcaatttgggaggccgaggcaggtgggtcgcttgagctcaggagttctagaccagcctgggcaacatggggaagccccatctctgcaaaataatacaaaaataattagccagacatggtagcatgcgcctgtagtaccagctactcgggaggctgaggtggaaggatcaattgagccggcaaggtcaaggcagcagtgagccatgattgcaccagtacactacagccgggtgacagagcaagaccctatctctcaaatgaataaataaataaatacatacattacttttcatttcagcattatttgtcatagggaactagaaacaatttaaggccaggcacagtggctcatgcctgtaattccagcactttgggaggccgaggtgggtggatcacttgaagtcaggggttcgagaccagcctggccaacatagtgaaactctgtctctactaaaaacacaaaaattagctgggtgtggtggctcacacctgtaatcccagctactcaggaggatgaggcacacgactcacttgaacccaggaggcagaggttgtacatcacgccactgcattccagcctggtcgacagagtgggactctgccaaaaaaaaaaaaaaaaaaaaggtaaagaaaaaagaaacaaagatagtagtaaatgatatttatttagggtaggtgtgggaactgggtatggcatatggagtaggagatttctcaccatcttatttaactttttgatttttaaaatggtaagatacacaaacatttatcatttttaccccttttaagtgtacaattcagtggcattaagtacattcactaattccagaacttattcatcaccctaaaatgaaactgcccattaagcagtcattccccacttccccttctcccaagctctggtaaccactaatctaccttctgtctctgtggatttttctagtttggatatgttccatatgtggaatcatacaatatgtgacctttcatgaatggttttgttcactgagcatgttttcaaggttcatccatgttgtagcatgtatgtatcagtacttcattttttttttttttttgagaccgagttttgctcttgttgcccaggctggaatgaaatggcacaatctcggctcactgcaacctccgcctcctgggttcaagcgattttcctgcctcagcctcccaagtagctagggttacaggagaccaccaccatgcctggataatttttgtatttttagtagatatggggtttcaccatgttggtatggctggttttgaactcctgatctcaagtgatccacccgcctcggcctcccaaagtgctgggattacagtcgtgagccactgtgcccagacccatttcattgctttttatggccaaataacgttctgctgtatggatataccaccttttctttatccattcatcagttgatggacatctgggttctttctaccttttagctattgtgagtagtgctgctgtgaacatttctgtaccagtttttgtttgaacacctgttttcaattcttctgagtatatacctaagagtgaaattgccaggttataggataatcctatgctcaatttattgaggaaccaaattgttttccacagagattgtaccactttacattccttacacggtaatgtataaagatcccaatttctccatgttctcatcaacactcattgtttttcattttttgatagtcattctagtagaagtgaagtggtatctcattgtggtttttttcttcttcttttcttttctttctttcttttttttttttttttttttttttgagacagggtctcgctctgtcgcctaggctggagtgcagtggcccgatcttggctcactgaagcctctgcctcccgggttcaagcgattcttgtgcctcagtttcccgagtagctggagctacaggcatgtaccaccatgcccagttaagttttgtatttttagcaaagatggggtttcaccatgttgcccaggccggtcttgaactcctgacctcaagtgatccacccacctcagcctcccaaagtgctaggattacaggtgtgagccaccatacccagatcattgtgggtttcatttgcattttcctaatgattaatagtgttgagcatcttttcatttgctgggtggctatttgtctatcttctttgaagaaatgtctttttaagtcttttgctcattttgaaagttgggttgtctttttgttgttgaattgtaagagttcgttatatattaaggatactcatctgatatataatctgcaaatattttctcccattctgtgggttgtcttcgctctcttgacattgtctttgatgcacaaaactttaaatttttgatgaagtccaatgtatctgtttttccttttgttgtttgtgcttttggtattacagctaagaatccattaccaagtccaagattatgaagatttacctatatactttcttctaagaatttatagttttagtagttatatttagggctttgatctcttttcagttaacctttgtatatggtgtgaggtggggagggggtccaacttcattcttttgcatgttgaaagcaccttttattttttgagaaaaagtggtggcgtgtgccactacacccggctaatttttgtatttttagtagagacggggtttcactatgttagccaggctggtcttgaactcctgaccttgtgatctgcctgtctcggcctcccaaagtgttgggattacaggcatgagccacggtacctggcctttttttaaagatggagtttcagctgggcatgtaatcccagctcatgcctgtaatcccagcactttgggagcctgaggcaggtggatcacttgaggtcaggagtttgagaccagcccggccaatgtggtcaaaccctctctctactaaaaatacaaaaactagtcgggtgtggtggcgcatgcctgtagtcccagatacttgggcgcctgaggccagagaattgcttgaatctgggaggcagaggttgcagtgagctgagatggcgcgactgcactccagcctgagcaagactccatctcaaaaaaacaaaacaaaacaaaaaagacagagtttcactctgttgtccaggctgcagggcagtggcacaattgcagctcactgcaacctctgcctcctgggatcatgtgattctcctgcctcagcctcctgagtagctgggattacaggcgcccaccaccacacccagctagtttttgtatttttagtagagacggggtttcaccatgttgtccaggctggtctcgaactcctgacctcaagtgatccacctgcctcggcctcccaaagtgctgggattacaggcatgagccactgtgcccagccccagccccattttttgaagacagtttttctcattgaatggtttggcacccttgttggaaatcaattggccacagaagcatagtttatttctggactctttcaattctattccactgatgtataatatatgtgtatccttatgccagtaccacactgtcttctttttttctttttaagagacagggtttcactctgttgcccagggcaaagtacagtgacacgatcatagctcactgcagcctcaaactactgggttcaagctgtcttctcaccagcactgggattacacgcatgagccactgcgaccagccaatgctgtcttgatcactgtagctttgcagtatcttttttacaagattattttggctatttggggttccgagcaattccatataaattttaggatcagcttttccatttctgcaaaaaaaaaagggccaatgggatttaactttttgacttatgaagcgtggtaaatgaattacctgctcattttaaaaaattaatatgtttttttaaaatcaggaatttgcttggctttcctgaaggtcactcaaggtattaccctgtctccaggctctttcttcaatctagattatctttgtgcaaagtacagaaagtagaaaatttttttgccaacactggcctttctccccttatcccagacctgcagagagttcttgtggtctaatcttcattcctcttgctaccatgtcaaccaagcagaactattcttccttgactctttcacacactccctcatctgctccagctttcttttctttaattcccagtcactgaccctcttctcttatttagaatccaagattgtaaaagccgccaagtatgtcagactatttattcaacttctccactttgcagatgtggaaactaaagcccagagagaaagtctgacttgccccacagccagtgagtgactgcagcagcaccagaatctggtctgtttcctgtttggctcttctaccactacggcttgggatctcggtaaatagaactgggtgcagactttagtgctttcaggctcagtccccaaatcctttagtaggccattttgcccaccttggccacatgaagtctgcacacaggaaggacagatcttctctgaggcccctattttttttttttttaagacagattcttgctttgtcacccaggctggtgtgcagtggtgtgatctcggctcactacaacctccgcctccctggttcaagcaattctcttgcctcagcttcctgagtaactgggattacaggtgcacgccaccatgcctgactaatttttgtatttttagtagagatggggtttcgccatgttgactaggctggtttcgaattcctgagctcaggtgatccacccaccttggcttcctaaagtgctgagataacaggcatgagccaccacgtccggaggcccctacctttgcgtgatacctgctccttcccattattttgaagaaactgctgaatccacaggggaagtgaaactatgaggggagataaagtgttggggcagagttttgagagtgataaccacaagctaagtttagaggactgagagtttgagagtcctccctgaggacccacatgtccccctttatatcctattacagggcatggtggctttgccaatggtccttgttttgctgctggtcctgagcagaggtgagagtgaattggacgccaagatcccatccacaggggatgccacagaatggcggaatcctcacctgtccatgctggggtcctgccagccagccccctcctgccagaagtgcatcctctcacaccccagctgtgcatggtgcaagcaactggtaaagatgggcctatggtctttgaaccatgtgtatgtgtatgtgtatgtgtatatgtatgtgcagcctggatatatgtgtgtgattgtgtgggcacatgtatgattttctgggaacactccacagtctcatgtgtgactgagtttgtgcacataggactcctgtgtgtggatatacgtgtgtgcatacacatgcatgcaagtttcttgtgcacttaggattatgtctgtatccatttgcttgtgtatatatggagagaatttgtgaccatgaattgggagaaagtatgtgcttgtgggaattcatgcttgtgtataggtagagggagctgttgtgtatgtaaacatgggagaaaggtactacccagtgcgggctgccataacaaaataccataagctgggtggtttaagcaacaggaattcatttctcctaatgttggaagctgggaagtccaagatcaaagtgctggctgatttggttcctcagttagggccctcttccttgcttgcagactggctaccttctcgctgtgtcctcacatagtggggatggggttgggggatgggggtgggaactgcctctcttcttgtaagcgcactaattccatcatgagggttccacccttaggccctcatctaagcctaggtacctcccaaaggctccatctccaaataccatcacatggagggttagggcttcaacagaggatggggtgggggtggggaggggtaggggtgcagagttcagtccatagcagcaagtgtgtggttagatttctaagtctgtgttttgggtggaggcattctgttcctgggtgtgtagtagcctatgactggacaggtagaaagggaaggggtggaagcatatgggcttgagggggactggaaaggcttgagctacctaagtggccaagaaggatgctagggagcaggtgtctccctggctcttcaaacaaagctgttgtttgtttatgctttaaagagacagaactcatggagcagcagagcaggaggggtgcccaagctgggatgggggcaggtctgcctctctcagtgtaagatggagggttagatcagtgtgggagcccagataagagatgggggcaaattcacatggaagcagagctacagatgcttagaggacccaggagaaaggcacacagggacccaggagaaaggcacacagtgaatctgactggacaagaagggtagatactgagtctgcacaaggcagagaggcagaggtgccaggctggagctggggagctggcgctcttgagatggtcaggtctgtgtctggcttctccagtctcctgacacctagggccagctgagaagccgctctaaccatttccttcccctgtggcttcccaaccagtcccagggataccagacatattggtggggaggacgcctcctcttctgttcttatcctttcctttccaaggagctgtgggtcttagtctgaccaccttcccattttggtgtgagcttgctggggaggttggcctaggaggcaaggcccctgtttttttgtggtagtgaagtctctaagatctcaacaggaagtctgtgactctattggggaggagaggggtaggtggctggggcaagggggagttgagataggggaggggtgggggactgctataggaagtggggctctgacaaggaagaaaggaccagagttccaccggtggcggttcatgcctgtaatcccagtactttgggaggctggggcaggaggatttcttggggccaggagttcgaaacaagactgggcaaaaaagcaagaccctgtctttacagtaggaaaaaaaaaaaaggcgtggtggttcacgtctgtaatcccagcactttgggaggctgaggtgggcgcacttgaggtcaaaagtttgagaccagcctgacctatatggggaaatcccgtctctactaaaaatacaaaaaacctgggcgtggtggcacatgcctgtagtcccagctaccgggaggctgaggtacgagaatcacttgaacccgggaggtggaggttgcagtgagcggagatagcaccgcagcactccagcctgggtgacagagtgagactccgtctcaaaaacaaaacaaaacaacaaaaaagaccggagttcaagggtagctgtggcgaggaagaggggagggaatcagggactcaaggagtggactggaggtgggggcattagaggtggccgggtttgcagggtgtttttgggagcggggcccagggcctatgcaggaccgggtcccgacgcgccttttcttgctgagcagaacttcaccgcgtcgggagaggcggaggcgcggcgctgcgcccgacgagaggagctgctggctcgaggctgcccgctggaggagctggaggagccccgcggccagcaggaggtgctgcaggaccagccgctcagccagggcgcccgcggagagggtgccacccagctggcgccgcagcgggtccgggtcacgctgcggcctggtgagttaggggaggcggggccaggccagaggtctaggctggcgttccaatgcgctctgctgacccgcccgccgcccaccccctaggggagccccagcagctccaggtccgcttccttcgtgctgagggatacccggtggacctgtactaccttatggacctgagctactccatgaaggacgacctggaacgcgtgcgccagctcgggcacgctctgctggtccggctgcaggaagtcacccattctgtgcgcattggtgagccgagcgctgcctcccgccctgttagcccttgcctattcaaccactgccctagcctctgccaacatcctggactcacaagggccccaacttgccctcccagttgttgacaggcctagccaggccacaggttcgttcctgcatctctccctagacacccatcttctaggtcagagctggagaggactcaagagactatccgttttacaggcctgagacccagcgtctggagagactgtgctcaggcactctctgggcctggacagctgccctgggcctaccaacctggaacctgccctgatgcaagtctccctggacaagacctagatgccactgtccctggctccacattctgtgccaactctcctctattcaaatttccttcctttgcctcatgctgctaccatatcctatacccgggccttcttccgccttctggaagttcccatttgcccaggaagttgtgtcctcctcttaatgtcttctacctgccacttccctgagccagtggtctcactgctttgcttgccagttaccaggtccctgccccctaggtgcaccctctcccatcaccgtcctccccaccccacttcccatagagcgctctcatgggtcctctgttgctgccctgcgacttctggcacaaccttcattcctctcctctcccaggttttggttcctttgtggacaaaacggtgctgccctttgtgagcacagtaccctccaaactgcgccacccctgccccacccggctggagcgctgccagtcaccattcagctttcaccatgtgctgtccctgacgggggacgcacaagccttcgagcgggaggtggggcgccagagtgtgtccggcaatctggactcgcctgaaggtggcttcgatgccattctgcaggctgcactctgccaggtgaggaggtggggcggggatctgagcctggaggtctgtttgggaacaggactgccccttatggttgtgtagtgcacagttctagggggtgccattcataccatagttgctgtagtttgtatatttagcaattacctggtgggttggagtaaaatatcttgaggaggaggcatgtttttcgaattcacaagtaagcctccactagcagcagccttctttagggggctggagagagcagggagaagcaaggtactggaggcaaggggatggacaagctgactcagagtgaggctggccacattcttggctcacagagctggctggggtggctcaagggccatgcccaggtgcatagcctaccacccccactgtaactcaggagcagattggctggagaaatgtgtcccggctgctggtgttcacttcagacgacacattccatacagctggggacgggaagttgggcggcattttcatgcccagtgatgggcactgccacttggacagcaatggcctctacagtcgcagcacagagtttgtgagtccctattgtcaccctgcctctccccatttcagccacacctctttccttccaggactcccactcccagccttacagtcctcaccagtaaccaccctgtagacaccctgccatcttctccagcagttcctcctttcagctgccgagacaggacacctttgtcaccccattccctacctccttcctattccatagcaagcctccagggctcctccagctggtggaaccttggttcctggattccctgggctgagggcaccttgcatgttgtgggggaaacctgtggggctcaccttgtccctgctccctgtcccaggactacccttctgtgggtcaggtagcccaggccctctctgcagcaaatatccagcccatctttgctgtcaccagtgccgcactgcctgtctaccaggtgagagctgtctacatgtcagatcccccagccccatcccaacctttctcaattcccccaaagatacattctccgtcccccatatgccctccttccagacctccgggagcccattttcctagagctgtgacccaccatttcccttaactccccaaaactcaggagctgagtaaactgattcctaagtctgcagttggggagctgagtgaggactccagcaacgtggtacagctcatcatggatgcttataatgtgaggggccaggggaaggggagatggtgagggtgggcactagggaaaggtggaaaggggcccttgggtggggtggcagaactaagaggaaatggtcactttttggtctaacattccacagctcctacctatagtctgcatgtggctgtcattaactgttcaatcttacattcataaatcttaaaacatctgacatgcagacagcatggagctctgctctgggcaagaaaggaaagtttcaatcactgagcttttatttcatgcgcccaccatcctaggtgtacatactgtttgctacttcatttaaccctgccaaaagccttgcttcataactgttaccatccctattttacagatgaggaaacccacacaaggaggctaagtgacttgtccaaggtcacaaagataacaaacagaagggctggcatttgtccccagtcagtctgtacataaacaatgtgagatctggagcctgctttcactaagtttataaataattcagagctattcttcacaagaaccatagaattttacaggtagaaggaaccataaaaattcttagtggtttaggtcttacctctgcaactaggctgtaggcttcttgataacagggagcatctcctaaacctatggcaagaaaatctgagacagtatcaaatttcccaagataaactctgtaatgaatctgccctttagacctggcatgcacccaggttatgtgtcctgatgtggggcttcacaaacatggtctctgtacatttacagcacctggccagcctgtgaccctcagtggcccttgctggcagctgcactgaaatgctgtattagaggcctctggatcctgtatgttctatgcaaggttctcacgggtcctccctccagaaatgcctcgactgggcagaatggaaggagccccaggtggcagagggcagggtgctggcagaattgggaacacctggagaggacataaggtggggttggagtccttttctgccttcacgtggttatccactcgctctcttcccatagagcctgtcttccaccgtgacccttgaacactcttcactccctcctggggtccacatttcttacgaatcccagtgtgagggtcctgagaagagggagggtaaggctgaggatcgaggacagtgcaaccacgtccgaatcaaccagacggtgagagccagcaagcacaggcagaaggcgggcagggggaggtgggagtgggaaattaagcaggcaggaaaagatgggtggcaggtacagggagtgacatgagagcctgaggtagtcctccatccatccttcctggagcagtctgagtggcttcctcaggcatgtttctaggagttgggtgttaacatgaccccatcccttcatttactaaggggtgaggttctggatgattggtgagtttggggtatgtgtgtgtgcatgtgtataaccatggcctgattcctgcctcttctcaggtgactttctgggtttctctccaagccacccactgcctcccagagccccatctcctgaggctccgggcccttggcttctcagaggagctgattgtggagttgcacacgctgtgtgactgtaattgcagtgacacccagccccaggctccccactgcagtgatggccagggacacctacaatgtggtgtatgcaggtgagggcctcctcttcccttcaaccacctggctcccaacaaccaaccaaccctggcccgtattcctccgctgctttccatcccccccacaggagtctcctcgacatcccgactccccactcagtttcatcccttgaagcttcccatgtatgtccaggccctccccctgactggggctccctccactctctcacccctcagtgtttcactatacttaacatcctttcagctgtttgtctgtctccaactcttttgagaacttgggtttagaggttcctgggagagacaagtcaggggtgatcaaaccctctgacctacttctctcctttgccttccccttccagctgtgcccctggccgcctaggtcggctctgtgagtgctctgtggcagagctgtcctccccagacctggaatctgggtgccgggctcccaatggcacagggcccctgtgcagtggaaagggtcactgtcaatgtggacgctgcagctgcagtggacagagctctgggcatctgtgcgagtgtgacgatgccagctgtgagcgacatgagggcatcctctgcggaggtacctggagccttggggagaagggcggggtctgagaggccttcccaaggcacttaggctctgggaatctcctggcatggaaggcaaatatcaaaagattccggtgtatgggttagagggtgtggttggcatacttcaaaaagattggatgcgtgggcttgtcgtgccacatgatggccaactgtgtgtgtggagtatggttgtcaccctctgcatggctctaccccaggctttggtcgctgccaatgtggagtatgtcactgtcatgccaaccgcacgggcagagcatgcgaatgcagtggggacatggacagttgcatcagtcccgagggagggctctgcagtgggcatggacgctgcaaatgcaaccgctgccagtgcttggacggctactatggtgctctatgcgaccaatgcccaggctgcaagacaccatgcgagagacaccggtgaggcctcaggtcttgcatcttgggagcaggggcacagtggggcttgttgagctactcccatctgcctactctccgtcctgcttgtagttgggcaaggggcattgggtgacaactgcccagctggtaacctattggcatctactacacagggactgtgcagagtgtggggccttcaggactggcccactggccaccaactgcagtacagcttgtgcccataccaatgtgaccctggccttggcccctatcttggatgatggctggtgcaaagagcggaccctggacaaccagctgttcttcttcttggtggaggatgacgccagaggcacggtcgtgctcagagtgagaccccaagaaagtaagtgggcagggatgccacaaaccctggaagtttgatgggcagggtctcaactctggggatgcagagtgaagataatgggaaacaggctgggggctgaagcagaactgtgaatccaggacaaaggcagaggcaattgagcaagtggacgaggctaaggccctgggcggtgaggataaccttggacctccctgggttccctttgctttcagtgacagacagggttggacaggctggacacactgtgagcaactgatgatctgtccctatgctccagctcccacaagtgtgtctggtttcttgttcacagagggagcagaccacacgcaggccattgtgctgggctgcgtagggggcatcgtggcagtggggctggggctggtcctggcttaccggctctcggtggaaatctatgaccgccgggaatacagtcgctttgagaaggagcagcaacaactcaactggaagcaggtgaggagacttcctggttaggcccctttttagctgttcccccaccacaagaccagccctgattcctcccactgggttcccccagcccctggcacatgtaaccaacccctctgctaacttccatcagctcctaggattcggctcaaggctggcctcttaggtctagaaaggggacgaggaatcctgggatttttgcttataatctgcaacatctttctccaggacagtaatcctctctacaaaagtgccatcacgaccaccatcaatcctcgctttcaagaggcagacagtcccactctctgaaggagggagggacacttacccaaggctcttctccttggaggacagtgggaactggagggtgagaggaagggtgggtctgtaagaccttggtaggggactaattcactggcgaggtgcggccaccaccctacttcattttcagagtgacacccaagagggctgcttcccatgcctgcaaccttgcatccatctgggctaccccacccaagtatacaataaagtcttacctcagaccaca

SEQ ID NO: 5 - protein, 798 aa, Homo sapiens
MVALPMVLVLLLVLSRGESELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDDLERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLCDCNCSDTQPQAPHCSDGQGHLQCGVCSCAPGRLGRLCECSVAELSSPDLESGCRAPNGTGPLCSGKGHCQCGRCSCSGQSSGHLCECDDASCERHEGILCGGFGRCQCGVCHCHANRTGRACECSGDMDSCISPEGGLCSGHGRCKCNRCQCLDGYYGALCDQCPGCKTPCERHRDCAECGAFRTGPLATNCSTACAHTNVTLALAPILDDGWCKERTLDNQLFFFLVEDDARGTVVLRVRPQEKGADHTQAIVLGCVGGIVAVGLGLVLAYRLSVEIYDRREYSRFEKEQQQLNWKQDSNPLYKSAITTTINPRFQEADSPTL

SEQ ID NO: 6 - protein, 650 aa, Homo sapiens
MVALPMVLVLLLVLSRGESELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDDLERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLCDCNCSDTQPQAPHCSDGQGHLQCGVCRDCAECGAFRTGPLATNCSTACAHTNVTLALAPILDDGWCKERTLDNQLFFFLVEDDARGTVVLRVRPQEKGADHTQAIVLGCVGGIVAVGLGLVLAYRLSVEIYDRREYSRFEKEQQQLNWKQDSNPLYKSAITTTINPRFQEADSPTL

SEQ ID NO: 7 - DNA, 16672 bp, Homo sapiens
gcttcctttctcgtgttgttatcgggtagctgcctgctcagaacccacaaagcctgcccctcatcccaggcagagagcaacccagctctttccccagacactgagagctggtggtgcctgctgtcccagggagagttgcatcgccctccacagtgagtattgctcttcttcttgttgcccccaacccagtgtcatcttggccctcagttgtgtgctgctatgatggcttctaaaacctgggcttgcactcatttgttcagaggtcatgggcaccaggcacttttctagaaacttccaggaaggtaaatgggaattggtgatgcccaacccaggtttcttgtagggcgagtaataaaactccctcccccttcacatacatgaagcttaggcttaaggcagtctgtgatttataccttaaaggaaggagaagcaggtgctgggggacaagatggtggccaggggtaaaggacacacccatgaggtccttgagtctgagacactgccccatggaaaggattctttgtagcagtgaaaaactggaaacaaggaaactatgcatcaggaggcgaatggttaaatgaattgtgatgtatctagacaatggaatacacagcagccatccaattctatgatacggttctatgttgaattggcatggaaggtgatccatgaaagatgggcaattagaaagcaagatagaagattatatgtgtagtctgatttcctttctattaagtgcgtgtgtgtctgtgtgtgtgtgtgtgtgtgtgtgaggttgtgttacagagaaaaagtcagaaaggaagtatctcagaaacttaacaatggctaaactaggaaagggtgcttcaggagtttttattttctatattatgtatgttggcattttggggattttttgtgcattgtaaaaatactcctttatgatcagagtaaagcaaaaagttattttaaatgactcatcagtggatgctggtttgccttttcaaacacacctaccctctttagttctgaattagctgttactccatagacggctggcaagtccatccagagagtggtcactgtgtgctgaaccggaagggctgtgcagcagccagtctgtgggatcccatctccttactccctttcctgaacgagcaaaagctaatttactatttcagcatctggaatttcggagtccttttcttccaatatctgcttatctgttctgaagcctctttgtgtaggcctcactcacaggcttgtgttgggagcccctgggccctgtgcagcccctgctgggcggttggcaggcatctgatccaggcatggtgagtggatggatctgatagatggctcagagaccctgcctcgtgtagggaacaaaaagcagagctatgaaaaaatgtaagagatgattcaagctccgtgtagcaaattgtacaaagttcaaagtaaggacctgcattcctgactcatgttagtatctgatttgatgttagtgctgtaagcatcagaggctataagtgtggggagatgctgtgtgcacaataaccccatgctgattttcatgttggctacgtaagaatgttagagaatacaaaaatagtgattggataataataggttccaagctaacttttctaataggcacttttctcttttggagaattagcagctctctcccttcctttatctcccaggtccctgcacataccccatgctttctgactttcatgccttagtgcatatgttttcccaccctggagcaatgttccctcccctcgttgcatgtctccatgctcttcggccttcaaggacccgcaaaaatgtcacctcttcaatgaagccctcctggattctccccagagctaaagacatgacctccttcttccaattcctaccacaactcatagcactttccctttgttgttatttgcagccatgacttactcagtgtatttgaatgcaagcttcttgaggacatggatcatgtcttacacattgtttaagccaaataaatgtatcaggtactcaaggcacattgaataagtgggttcgatggatggcccatggcacctgcaacaaggcctcactgagcagcaagggagagatttgccacagtgctgagtcaactgcccatccactcccgacagaccgctgagatggtcctcactctcagtatgtttggtcatatgtgggattttcccccctatatttttcccgctttctcactggcaagacaaaaatgaaagcaagcactgagacttagtagctgggagaataaaatatttgagaagtattctgacaaaaatgaaagagaaatagatggcaaggctgtctgtggaggttctagatgtgggctctagggaggggtatgtcctgtttttactctttggctctctccaaattttctatgagccagaagcagatgcatattttataaactgtaatatattacattctattatacaccataatatattacatgatatatactatttgtttatttattttagagacagggtcttgctctgtcactcaggctgcagtgcagcagtgcaatgttagctcacttcagccttgaactcctgggttcaagcgattgtcctgcgtcagccagctgagtagctgggactacagggtaggcaaccatgcctggcatatttttaaattttttgtagagatggggtcttgctctgttgcccaggctgatctcaaattcctggcctcaagtgatgctcccacctcagcctcccaaagtgttggaattacaggcatgagccatcttgcctggcttgtgtttatatattacctttaaaaaaaaaaataaagagaaacctttatagaaagctagtgtacaacttagttgcataaagtagcctcggtgaatgtaattactagacttgctctttctgcggtagctggagcaggctacaagttcctcctctagatgagttttgtaaacaggtgtgctgaacgttagagcattgggcttcttcttcttttttttttaaatctgacaagttatttttatgacagctcttaaaataaaattttacacacttggtaagatatttaaatagtttgcaaaatctaaatgaaaacatgaaagttcattttccactggtcacagtggttcagtctccttccagaaaatgtttcggtgctggtgagcagtgcagtgtatgtctttttctatttttttttttttaaagatcacacaacataagtcttcctacatgttgcattttaatcttatagtcttgaggacctcttttctatcagcatgaggaccatccttgtttattggctacacatggtttattggattgatgtgtcataatattaccaattgctgaatatttaagttgttttccaatttttgttctgctccaacatatatccctatacctggacccttgtgttcttttgtgagaataccagtagggcaaattccaagcagatgcgctgcagagtcagagatgttgtgctttgtaaattttgacagatatggcttaaccctccaaaaagatcacataaatttatactcctcacagcagtgtggaggccttttttccaaactcttaccaatgtcgttaccatctattgctatttccaatctgaaacttgctgtcactcactataacaaagccattctttcttctttttgtggtttctttctttgggaatttttctcaggtattacatgattaatattcagtcctttcatgcaagcaaaaaggaggtgacataagccctgggtattagaaatatatatataacatatatatatatttatataaatatatatataacatatatatatatttatataaatatatatataacatatatatataacatatatatatatataacatatataaagtgttttctaattgatatctcaaaaatcaacctccgtattccccaagctgtaataaaataaaatttgctgagttgaggtcttccaaactcaactgggcatttttcatctggggcaaaaaaaagaagagtaatgctcagctccaaagtggctgcttttctgcttgctgggtgagtttcttatttcaaaacagaggctcactgggcttcttgatggcacctggagtcatgccaagcaggaagcaatcactccacagagctgaagatgtcagaaccgttcagaaatactgcctggggaaagttcttggttcttcggcttaggaggaaaggggcagaatacaacctttctgaagaaaatgcatctttcattcacctgagaaatatttcaaaataactataaaaagaagttagaagcaggtgaattgccccacagaggggaatgtttaaatgaattggggcacagcccctgtcggaatgccaggtagctgatgccttcacctgtctgaagaatgtttaatgatgaaagaaatgttcataatccgttaggtgggaaaaagaaaataaaagttcaaagtgtacgaatgcatagaaacacggctgggagggaaaacaacagaaagttaaaagtggttattttagggtaggaagattacgggtgattttgattttctcctctcggcttttctgtatttgtcaaatttcctgcaaacacgtgtgttacttttgtaacaaaaaaagtcatttttcagataaatgtcagtgttttcctggcccccagagggctgcagtgaaattcggtgaatgcccaggtggctgggtaaggtcacggccggaggctggggctactcatttttatttcagacacagtcctgttccaggctttcttgtgtcttagggtcaattgccaaaatatttttttctcttctaaaaatttattgtgagtcttacttgtatctaatttcaagctcactgaaaagtgaaaaattgtgtaaagaatgcttataaacccttcacccaaaccattgttttcattttgtcctactggctttattattctccttctctctatatacatatgttttcattaattttttgcattttttgcttattttttgaatcctttgagaatatgccaaagacactgttctcttttgcccctaaatacttcaggggataattcctaagaacaggggcattctcctgtaaaaccacaatgcagtcagcaaaatcaggaaatttaatccggatgcagcactatcatcgaattcacagcacctattgtaatgcccttaatttcccagtaatgtccttcacaattattttttttccccactccaggaccaaatccaggttcttgccttgcatctacttgtcctgtctctttagtctccttcaacctgagatggtttctcagtttttctttgtctcacttgaccttgatatttggccaattactttgcagaaagtccttcattttgcgtttttctgatgcttcctcattctttgatttaggttgaccttttttccaggaataaagatgatgtgtcttccctcctcaactctttaggacaaatgtgacactggtttgcttccatgttggtggtatggggtttgaagaccaacttagctggtcttcaaatccaacaccatcaacatgtagttacatgtctccaaggagccctggctgtttagtggagaaagatatttagattccaagatcagggcactaggtgctaattatgctgtgtatcattgcttttaggtcctctcagcagacagagacaggaaatatgtatgtgaaacacacatacatatacatatacaaattgtctatctatctatctaatttaatcttaaaaacatatgggtataaagacatgtgaacatgtatgttcattccaacactattcacaatagcaaaaacatggaatcaacctaaatgttcatcaacgatagactggataaggaaaatgtggtacatatacatcatggaatactacacagccataaaaaagaacaagatcctgtcctttgaagcaacatggatggaactggaggccattatcctaagtgaattaacacagaacagaaaaacaaatgtcacatgttctcatttataaatgggagctaaatgttgagtacatatggacacaaaaaagggaataacagacactgggacccccttgaggatggagggaggtaggagggtgaggataaaaaaactatctaccagtcaggtactatgcttattaacctgggtggcgaaataatctatacaccaagcccccatgacgcgcaatttacctatatgacaaacctgcacatatacccctgaagctaaaataaaaggaaaaaacccacaaaaacaaaccaaaacaaaaaaaacccacgtgggtccgtagtgatctctcaagttggctacaacagcacaaggcactccagtgcacttttgaaaagctaccagcagagaggttaccaggaggaagttctggttgctttcctcctcagggcctgctgacagccggagatgacactgagaagtgaagtgccagaaggttttcatcgtactttgctttttcctgccttacttgaacagctttattgaggtccaattataataaactgcaaatattaaaatgcagaatgtaagttctgacgtacatacacccttgcgaagctctcacacaacccagacagtgaacatatttatcccccactgaggtttcctttgtacccacctgccctcctgccctcccacactccccttccctctctctctctctctttttttttttgtttgagatggagtcttgctctgtcacccaggctggagtgcagtggtgcaatcttggctcactgcaacctctgcctcccaggttcaagtgatcctcctgcctcagcctcctgagtagctgagactacaggcgcacaccaccacacctggctaattttttgtatttttatagaaatggggtttcaccatgttggccagactggtctcgaactcccaacctcaggtgatctacccaccttggcctcccaaagtgctgggattataggcgtaagccaccatgcccagccacctccccttctctctctctctattgccaggacatcagtgattctgctttccgtcagtatagattagtttgcattatttagagttttatacaagtgaaatcatacagtatgaactccctttggtctggctacttggcataattgtttggagacttgtccatgttgctgtgtgaatcggtcattcattccctattccattggacggatatttcacagttaacttatccatttactacttgatggacatttaggctgttagcagtttttgcctaccacagagtaaagctgtaataaacactcacatactagtctttatggggacatatacttacaattttcttgggtgaatacttaggagtgggatgactggatcacatggtgagtgcatgtataacgtcttaagaaactgccagttttccaaagtggttttaccacacattatgcattggaaaccactgtactttcagaaaagcagggtggggtcatgatgttgttatggaaaggaagccaacaggttggctttataggatttatgaaagaggctatttttttcgagaaccttggggtgtgatgctggacaggtttccttgaaatgtactcgctaagggtataaaaggaaatgccccagctcaggcacagacttcgtcctcgaaggcagtcaagctgttgtttctttggttatgtaggggtgggctttaaagaaattcaaaacacatgaaaatgacaactcgagatttatttcagtcagttccgaggccctgtgcattagggaactagaactcattttcttcagtacacaaacctaggacttccttttcctgctgaagtcagggtaaagaggagaaaggtgggttctgggggcaccgggtcttttagggaggggccccaaccccagtgggcaccacccagccctcatgttcttcttgccacctggttactcaaaggcccttttcctggggagtccagagcccagggcattctcaaagcgaccactgtcctgttagcttcggtgagtcccttagctgatagagcagggggaaggggaaccagtcagcaaaacaggacttgacagacaagctgaatcaagggaaagggagctaatgtagaaaggatgagctgggaaaaatgtaacagcccacctgccattcctcatttgtggatgggagatgtactaattattgtctaagtagaaagaaaacaaatgctagaatcaggtgaccctcagagtcaaagaagtgaatgataacttcaaaggagaggttcattcatttctgtgtggactactcagatgattctgaaatctagaatactatatagactctaactcctgcttggaatatttttttcagaagaataagcagatttaaaatttaatctccatctttttggcatttggttgttactattcgtttacaagccagtccactttttgatttttgtcccttttccaggagcaggcttgcatctgactgacccaccatgacacccacagacttcacagtgagtacagccgtgctcctctggctcctcaaaacacacactcatcttccctttttagggggtgtgggaaggatccactgtgggggaaggatttatctgtgtggtttcccagggtaagatctctgcctggcaatgcgcattgctgggtaggttgttgtccagcacagagggttttgcaaatgcaaagaggcagctatgctttggggtatgttgtggagacagaggaaaatgtggtgttataaacagctaaggatttttaaccatctttaactagggtgtgcttagttccatatgcctgttctaaaactcagacaaatgaggcaaagggagaattcatgtaatttgcttttaaaaaacccatggtgggcacattagttgtatttcttgcataacattatgtagaccttatgaaaacacgtaacagtatacattcccctgaaagagaccaagagctgagcaaaaattagtgagtcagtctgggcgtggtagctcacgcctgtaatcccagcactttgggaggcagaggcgggcggatcacctggccaaggccaggatttcaagaccagcctgcccaacatggcaaaaccccatctctactaaaaatacaaaaatagctaggtgtggtggcatgcgcatgtaatcccagctacgtgggaggctgaggcacgagagtcgcttgagcctgggaggtggaggttgcagtgagccaagatcacgccattgcactccagcctgggcaacagagcgagactctgtcttaaaaaaaaaaaaaaaaatcagtgagtcaaatcataatccagagaatctttttttcttttacagccgaatgtaattaagtgtagaatcaaattaatttcccatgcttttgtaacattcataaatgccttataaaattacctaagggctttgattttgctcttctccaagggaaagcctgggttttacatgtagataattgaaatccagacttcctagcaggcgggctaacaaactgcctcacagtacattgtgggtttccatgatctatggagagtggtggttgaaacagtttatcaactaaattatttatgatatgctaatgattcattagtattacatatgtatagttacctaattgcacctatatccataacaacaaaaaagtaattaatttggtcctggtgaagtaactattattctgacaaaaagtagactcttcagtttctcctgcattaaattcacctttcaatccctgaggctgtggaaacagaagtagatgaaggcagcagagccagggctgcttctgagcttgccagtcatttgcttttatcatttgggatcttggataatagccaactctgcaaagcagccccagataagaaggaaactgcacctcagtaatgacctgctcttgtcagaggagtctggcgccatctaaatgggaagaagatgggacccctgtggccctgactcccatattccatggaaagtagccgtggcaccctgtggccacctttctgaggatgtgctgggccagtcacccatgcaggtgccccacattccctcagcgtcacagtaaaggcagagtcctttttacagatgaggagaccgagactggggaaggtggtttgagggttcaaggtctcatagaaatttggaggaatagcccttgaggggtgctgttggcaatcatagctttgctgggttcctccctcagggtgaatgtcaaagcactgaaatccaggtctcaatgttgggatttaatactttctcaagtgtggggccaaggtgtctgccagagtccctgctgataacaaattcattttttccccacctccctccttcttgtgccctctcttattccatctcccccattgccttgtgcccattcttggtgctaaggcacaatggaagccttggcaagcacagtccctgccctttctctgtccattccccagagaggggcatcttgagcctgggtttggctgaggatttccagagaacacatgagccagagaaggaggccctggaaagctctgggaccttcttcctgctctgggtgttcacaactgggacgcctgggctgctgcttctgagcccaggactcctgagggaagaggacgtgcctgtgtgggagcaacagcctgtgggctcagaggcttgccaggacacagacaggcaggatgggccccttgccctggctgtgcactgaggtttgtttacatttactgagggatttccagaggatttgatgtctaagatccatcaggcatgtgcaaaactatgagtgtgagtcatgacacagggggtgttgttccaggattcctctcgtggtcctgacaaattttatataaatctagatttggggtgtaaaattcaatcacttaaaaaatatgcctggggagtccctaacctaactaactaagtaacttcatgcaaaagcacgaggtccttaattctcacccagcagaaaaactatgccccctgggcttccagcaggacacaatgtccttgtctgggattcagtcttgggagtgttagctgtgcctgctcaccgggcagtggagaaagctgggtcacccaggtcctgggatttctgcacaatttctcccattctgctcctgcagtctcctccaggccccgctccagatcaccttccctcgctggcccaggaatccatctccttccaggaccttagcccaggactaacacagctaagtgatgctggccttcccacctgccccctctcatttgttccccaggaaccaaagtcgtcccttgtgcttgtccttctgccaagcatgccctctttcctcccttgccttcttctttcttccttgtctgtcttcctcttaacatgtctttaaaatccatgctgctcctcgctacctggagctgcagcaggaggagagcaggcttgctggaccggagaagcaactccaggcagcaagacccctgggccctcatctgtgaggctgcttgcgatttgcttcacagccgtgggctcaggctcatgtgagagtctattaaagctatttgaccaaaaaaaaaaaaaaaaaacacacaaaacacaaaacaccaacccacaaataacaaaaaaaaaacttcaaaagcaagtttagcacttattcattgtgtggattcacagtcggtacttgctcatctgcattgttttactgagattcctccaaaaggtcatcctcccaacctcttaaacctgctgtgatgtccactgtggggtctccatgctcttcacagtcttccttctattctcaagttcttctttcactcaaagtccactcaaacctcacctcctacaggacgactgtcttagtggggccatgattgcagctggttcccgtgtccagcgttgggaggattctcctggcatgtttctaaaatgtgtgtcacctaggaggtctgtcctctcgagagactgggtttccagacaagggccaatgctagaagaaacatctctaagtgtaaactgaaatggattaatcaagccaagggactaatcacatataaaatagggagacgagttttctttagttctttgcgtttgtgggccatttccagctgccctggcctgaagagccgcctccaggatcctgctcctgggtccctctctttgcaataggattttctcttgctcacatattggagatggagtcaggactagatgtcagattttcccagttgtcatccaagttaccttgggtcctcctgctacttcctgccagccagggcccaaataggactaacatctactgtcctttatcaatcaattcagtgagccctgatatctaagaagccctccacaaacacacacacaacaggttctatgttccaataaatttagaaaacattgttaaacaaaattcaacagattctgaaggatttctcagagtctttactgtgctaatgtgcaattaaaataaaacaagttctgggccgggcatggtggctcacgcctgtaatgccagcactttcggaggccaaagtgggtggattacgaggtcagaagttcgagaccagcctgaccaacatggtgaaatcccgtctctactaaaaatacaaaaattagccaggcatggtggcacacgcctgtaatcccagctactcaggaggctgaggcaggagaatcgcttgaacctgggaggcggaggttgcagtgagccgagatggcgccattgcactccagcctgggaagcaagagcaaaactccgtctcaaaacaaacaaacaaataaataaaacaagttttgtaggacttttcagatccttcaatgtgctcaggagtattgtgaatttcaaagaaggaatatgcaaagattctcaactattttaaccattagtgttccacctaaaacatggtttgggaaatgctagacagatctactagaaatagatggatattacaatgtatacagatagggagagcccagctaacaggaggtagagaatattttgctatttaaatataaacattcaagtacacaaaatcatctttgtacttatatatttgttaatattaactgttcaaaacaaaataaaaacttattgtggatttggggtgacttctagtccaaatgtatccagatgaattgaactattgcatttgcctcaggataaataaaagtagagatgagatttggcaaggaatgtccctcaaggaaagggagtgagagctggtagtccctggtggaatttgggagtcctaatgggagccctcagaaggaaactggtgggaagtgagagcgcttgaagatctgtgggcagaggttttggaaggaatgtagcaggcatggcacggactctcccctcctcctccatcagcagtccacatccttcattacctcccaccccaggtttgactcaagggcttacatctcagggtctcatttccagacctcatttcctgtttccgtagcaggggttttaggatggcttctgcttcaggaacccagaaaaagtgtaagaagtgtgtgtgtgtatgcgcatatatgtgtgtatatatgtgtgcatgcatgagtgtgtacctatgtgtatgtgagcatgagtgttggcatgtttgtgtacgagagcatgtgcaagtgcatgtattatgtgtatgcatgtacatatgagtgtgtgtgcttgttggggccagcttcatggctgtgcaacccacacagtcacataggggccctgtgcttagaagtgctgcttgtttgattgaatgctggatgtgttgaaattcctaataatttttggaccacgggtcctgctaactatagagcaggtcatggtgcttgtgtgtgtatgtagggttgagagtgtctgtgtgtgtatgtgtatgtgcatgtgtatgtgggtgtatgctggtgctcccggagctttcaggaaactcaggggactgggatccagatgagtttaagagcccttgctaacctttttagggtcagtgaaaaactgaatagataggagaagtacgatcacagtcatatcacagtttgataaaactgcccatcagtagtgtggaggcactcttaccccatcagagcacttggcatgcacactataaatgttcatttgcgtgtctggttgctctatgggtaggtaagtttcctgagagtgaagccacacatctgacttatttattatggtttctttggcacatagcacagtagccaacatacagtatgtgcttaataaacatctgttgaataaataaatatgtcacaacccaagcagatgtcctcagaatgcctatgtgtctttggccttatcataggtgtttggggttggaagggtcaagaggtccatgcctctgccatcagacaggaccttcaaaatattttccttgacctaatgccatcttgtgtccccttgcagagccctattcctaacatggctgatgactatggctctgaatccacatcttccatggaagactacgttaacttcaacttcactgacttctactgtgagaaaaacaatgtcaggcagtttgcgagccatttcctcccacccttgtactggctcgtgttcatcgtgggtgccttgggcaacagtcttgttatccttgtctactggtactgcacaagagtgaagaccatgaccgacatgttccttttgaatttggcaattgctgacctcctctttcttgtcactcttcccttctgggccattgctgctgctgaccagtggaagttccagaccttcatgtgcaaggtggtcaacagcatgtacaagatgaacttctacagctgtgtgttgctgatcatgtgcatcagcgtggacaggtacattgccattgcccaggccatgagagcacatacttggagggagaaaaggcttttgtacagcaaaatggtttgctttaccatctgggtattggcagctgctctctgcatcccagaaatcttatacagccaaatcaaggaggaatccggcattgctatctgcaccatggtttaccctagcgatgagagcaccaaactgaagtcagctgtcttgaccctgaaggtcattctggggttcttccttcccttcgtggtcatggcttgctgctataccatcatcattcacaccctgatacaagccaagaagtcttccaagcacaaagccctaaaagtgaccatcactgtcctgaccgtctttgtcttgtctcagtttccctacaactgcattttgttggtgcagaccattgacgcctatgccatgttcatctccaactgtgccgtttccaccaacattgacatctgcttccaggtcacccagaccatcgccttcttccacagttgcctgaaccctgttctctatgtttttgtgggtgagagattccgccgggatctcgtgaaaaccctgaagaacttgggttgcatcagccaggcccagtgggtttcatttacaaggagagagggaagcttgaagctgtcgtctatgttgctggagacaacctcaggagcactctccctctgaggggtcttctctgaggtgcatggttcttttggaagaaatgagaaatacagaaacagtttccccactgatgggaccagagagagtgaaagagaaaagaaaactcagaaagggatgaatctgaactatatgattacttgtagtcagaatttgccaaagcaaatatttcaaaatcaactgactagtgcaggaggctgttgattggctcttgactgtgatgcccgcaattctcaaaggaggactaaggaccggcactgtggagcaccctggctttgccactcgccggagcatcaatgccgctgcctctggaggagcccttggattttctccatgcactgtgaacttctgtggcttcagttctcatgctgcctcttccaaaaggggacacagaagcactggctgctgctacagaccgcaaaagcagaaagtttcgtgaaaatgtccatctttgggaaattttctaccctgctcttgagcctgataacccatgccaggtcttatagattcctgatctagaacctttccaggcaatctcagacctaatttccttctgttctccttgttctgttctgggccagtgaaggtccttgttctgattttgaaacgatctgcaggtcttgccagtgaacccctggacaactgaccacacccacaaggcatccaaagtctgttggcttccaatccatttctgtgtcctgctggaggttttaacctagacaaggattccgcttattccttggtatggtgacagtgtctctccatggcctgagcagggagattataacagctgggttcgcaggagccagccttggccctgttgtaggcttgttctgttgagtggcacttgctttgggtccaccgtctgtctgctccctagaaaatgggctggttcttttggccctcttctttctgaggcccactttattctgaggaatacagtgagcagatatgggcagcagccaggtagggcaaaggggtgaagcgcaggccttgctggaaggctatttacttccatgcttctccttttcttactctatagtggcaacattttaaaagcttttaacttagagattaggctgaaaaaaataagtaatggaattcacctttgcatcttttgtgtctttcttatcatgatttggcaaaatgcatcacctttgaaaatatttcacatattggaaaagtgctttttaatgtgtatatgaagcattaattacttgtcactttctttaccctgtctcaatattttaagtgtgtgcaattaaagatcaaatagatacatt

SEQ ID NO: 8 - protein, 369 aa, Homo sapiens
MTPTDFTSPIPNMADDYGSESTSSMEDYVNFNFTDFYCEKNNVRQFASHFLPPLYWLVFIVGALGNSLVILVYWYCTRVKTMTDMFLLNLAIADLLFLVTLPFWAIAAADQWKFQTFMCKVVNSMYKMNFYSCVLLIMCISVDRYIAIAQAMRAHTWREKRLLYSKMVCFTIWVLAAALCIPEILYSQIKEESGIAICTMVYPSDESTKLKSAVLTLKVILGFFLPFVVMACCYTIIIHTLIQAKKSSKHKALKVTITVLTVFVLSQFPYNCILLVQTIDAYAMFISNCAVSTNIDICFQVTQTIAFFHSCLNPVLYVFVGERFRRDLVKTLKNLGCISQAQWVSFTRREGSLKLSSMLLETTSGALSL